SEQUENCE LISTING (33 sequences)

SEQ ID NO: 1 - DNA, 29882 bp, SARS-CoV-2
  source 1..29882: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1269 aa, synthetic construct
  REGION 1..1269: note = Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 180 aa, SARS-CoV-2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 5 - protein, 180 aa, synthetic construct
  REGION 1..180: note = Recombinant protein
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTAASSGVTRELMRELNGG

SEQ ID NO: 6 - DNA, 17 bp, synthetic construct
  misc_feature 1..17: note = Primer
  source 1..17: mol_type = other DNA
gtaaaacgacggccagt

SEQ ID NO: 7 - DNA, 36 bp, synthetic construct
  misc_feature 1..36: note = Primer
  source 1..36: mol_type = other DNA
taagaacggtagtgagggagctggtggccatagtta

SEQ ID NO: 8 - DNA, 36 bp, synthetic construct
  misc_feature 1..36: note = Primer
  source 1..36: mol_type = other DNA
caccagctccctcactaccgttcttacgaagaagaa

SEQ ID NO: 9 - DNA, 40 bp, synthetic construct
  misc_feature 1..40: note = Primer
  source 1..40: mol_type = other DNA
aaaactggaacactgccgccagcagtggtgttacccgtga

SEQ ID NO: 10 - DNA, 42 bp, synthetic construct
  misc_feature 1..42: note = Primer
  source 1..42: mol_type = other DNA
gggtaacaccactgctggcggcagtgttccagttttcttgaa

SEQ ID NO: 11 - DNA, 20 bp, synthetic construct
  misc_feature 1..20: note = Primer
  source 1..20: mol_type = other DNA
cacgagcagcctctgatgca

SEQ ID NO: 12 - DNA, 35 bp, synthetic construct
  misc_feature 1..35: note = Primer
  source 1..35: mol_type = other DNA
actcagactaattctcgtagtgtagctagtcaatc

SEQ ID NO: 13 - DNA, 35 bp, synthetic construct
  misc_feature 1..35: note = Primer
  source 1..35: mol_type = other DNA
actagctacactacgagaattagtctgagtctgat

SEQ ID NO: 14 - DNA, 20 bp, synthetic construct
  misc_feature 1..20: note = Primer
  source 1..20: mol_type = other DNA
cagcatctgcaagtgtcact

SEQ ID NO: 15 - DNA, 20 bp, synthetic construct
  misc_feature 1..20: note = Primer
  source 1..20: mol_type = other DNA
ttaattttagccatggcaga

SEQ ID NO: 16 - DNA, 35 bp, synthetic construct
  misc_feature 1..35: note = Primer
  source 1..35: mol_type = other DNA
tttgcttgtacagtaaacgaacaaactaaaatgtc

SEQ ID NO: 17 - DNA, 35 bp, synthetic construct
  misc_feature 1..35: note = Primer
  source 1..35: mol_type = other DNA
ttttagtttgttcgtttactgtacaagcaaagcaa

SEQ ID NO: 18 - DNA, 20 bp, synthetic construct
  misc_feature 1..20: note = Primer
  source 1..20: mol_type = other DNA
gaagtccagcttctggccca

SEQ ID NO: 19 - DNA, 17 bp, synthetic construct
  misc_feature 1..17: note = Primer
  source 1..17: mol_type = other DNA
gtaaaacgacggccagt

SEQ ID NO: 20 - DNA, 36 bp, synthetic construct; identical to SEQ ID NO: 7
  misc_feature 1..36: note = Primer
  source 1..36: mol_type = other DNA

SEQ ID NO: 21 - DNA, 36 bp, synthetic construct; identical to SEQ ID NO: 8
  misc_feature 1..36: note = Primer
  source 1..36: mol_type = other DNA

SEQ ID NO: 22 - DNA, 40 bp, synthetic construct; identical to SEQ ID NO: 9
  misc_feature 1..40: note = Primer
  source 1..40: mol_type = other DNA

SEQ ID NO: 23 - DNA, 42 bp, synthetic construct; identical to SEQ ID NO: 10
  misc_feature 1..42: note = Primer
  source 1..42: mol_type = other DNA

SEQ ID NO: 24 - DNA, 20 bp, synthetic construct
  misc_feature 1..20: note = Primer
  source 1..20: mol_type = other DNA
cacgagcagcctctgatgca

SEQ ID NO: 25 - DNA, 17 bp, synthetic construct
  misc_feature 1..17: note = Primer
  source 1..17: mol_type = other DNA
gtaaaacgacggccagt

SEQ ID NO: 26 - DNA, 35 bp, synthetic construct; identical to SEQ ID NO: 12
  misc_feature 1..35: note = Primer
  source 1..35: mol_type = other DNA

SEQ ID NO: 27 - DNA, 35 bp, synthetic construct; identical to SEQ ID NO: 13
  misc_feature 1..35: note = Primer
  source 1..35: mol_type = other DNA

SEQ ID NO: 28 - DNA, 20 bp, synthetic construct
  misc_feature 1..20: note = Primer
  source 1..20: mol_type = other DNA
cagcatctgcaagtgtcact

SEQ ID NO: 29 - DNA, 20 bp, synthetic construct
  misc_feature 1..20: note = Primer
  source 1..20: mol_type = other DNA
ttaattttagccatggcaga

SEQ ID NO: 30 - DNA, 35 bp, synthetic construct; identical to SEQ ID NO: 16
  misc_feature 1..35: note = Primer
  source 1..35: mol_type = other DNA

SEQ ID NO: 31 - DNA, 35 bp, synthetic construct; identical to SEQ ID NO: 17
  misc_feature 1..35: note = Primer
  source 1..35: mol_type = other DNA

SEQ ID NO: 32 - DNA, 20 bp, synthetic construct
  misc_feature 1..20: note = Primer
  source 1..20: mol_type = other DNA
gaagtccagcttctggccca

SEQ ID NO: 33 - DNA, 24 bp, synthetic construct
  source 1..24: mol_type = other DNA
actggaacactgccgccagcagtg

(19) United States
(12) Patent Application Publication
(10) Pub. No.: US 2025/0325653 A1
(43) Pub. Date: Oct. 23, 2025

(54) LIVE-ATTENUATED SARS-COV-2 VACCINE

(71) Applicant: The U.S.A., as represented by the Secretary, Department of Health and Human Services, Silver Spring, MD (US)

(72) Inventors: Tony T. Wang, Centreville, VA (US); Shufeng Liu, Ellicott City, MD (US); Charles B. Stauft, Columbia, MD (US); Prabhuanand Selvaraj, Gaithersburg, MD (US); Christopher Z. Lien, Silver Spring, MD (US)

(73) Assignee: The U.S.A., as represented by the Secretary, Department of Health and Human Services, Silver Spring, MD (US)

(21) Appl. No.: 18/868,378

(22) PCT Filed: Jun. 2, 2023
(86) PCT No.: PCT/US2023/067862

§ 371 Date: Nov. 22, 2024

Related U.S. Application Data
(60) Provisional application No. 63/348,850, filed on Jun. 3, 2022.

(51) Int. Cl. A61K 39/215; A61K 9/00; A61K 39/00; A61P 31/14; C07K 14/005; C12N 7/00

(52) U.S. Cl. CPC A61K 39/215; A61K 9/0043; A61P 31/14; C07K 14/005; C12N 7/00; A61K 2039/5254; A61K 2039/543; A61K 2039/545; C12N 2770/20022; C12N 2770/20034; C12N 2770/20051; C12N 2770/20062

(57) ABSTRACT

Engineered SARS-CoV-2 variants having a combination of attenuating modifications, and their use as live-attenuated SARS-CoV-2 vaccines, are described. The recombinant genome of the live-attenuated SARS-CoV-2 encodes a modified spike (S) protein with a deletion of the polybasic site (ΔPRRA); encodes a modified non-structural protein 1 (Nsp1) with K164A and H165A substitutions; and includes a mutation that prevents expression of open reading frames (ORFs) 6, 7a, 7b and 8. The disclosed live-attenuated SARS-CoV-2 retain the capacity to infect and replicate in mammalian cells. Immunogenic compositions that include a live-attenuated SARS-CoV-2 and methods of eliciting an immune response against SARS-CoV-2 in a subject are also described. Further disclosed are a collection of reverse genetics plasmids that include the complement of the recombinant genome of the live-attenuated SARS-CoV-2 and methods of producing a live-attenuated SARS-CoV-2 using the reverse genetics plasmids.

### CROSS REFERENCE TO RELATED APPLICATIONS

This application claims the benefit of U.S. Provisional Application No. 63/348,850, filed Jun. 3, 2022, which is herein incorporated by reference in its entirety.

### FIELD

This disclosure concerns modified SARS-CoV-2 containing a combination of attenuating mutations and use of the modified SARS-CoV-2 as a live-attenuated vaccine.

### INCORPORATION OF ELECTRONIC SEQUENCE LISTING

The electronic sequence listing, submitted herewith as an XML file named 9531-108350-02.xml (72,759 bytes), created on May 25, 2023, is herein incorporated by reference in its entirety.

### BACKGROUND

The rapid development of multiple vaccines has afforded powerful tools to curb the severe coronavirus disease 2019 (COVTD-19) pandemic. Among ten vaccines granted emergency use by the World Health Organization (WHO), seven of them, including the Pfizer and Moderna mRNA vaccines, three adenovirus vector-based vaccines (AstraZeneca/Oxford, AstraZeneca/Serum Institute of India, J&J), and two protein-based vaccines (Novavax and Covovax with Novavax formulation), express SARS-CoV-2 spike protein as the immunogen. The other three inactivated, whole virus vaccines (SinoPharm, Sinovac, and Bharat Biotech) are less immunogenic at inducing neutralizing antibodies. The efficacy of existing vaccines in preventing symptomatic infections, especially against new variants of concern, declines considerably over a period of six months. The stringent storage conditions and requirement of medical supplies to administer the vaccines put additional constraints on the worldwide distribution of some vaccines. For these reasons, a need exists for new vaccines that are not only potent, broadly protective, and elicit durable immunity, but are also easy to administer, store and transport.

Live attenuated viral vaccines (LAVs) utilize a living but weakened virus as immunogen. There are many examples of effective LAVs, including the measles, mumps and rubella vaccine, the oral polio vaccine, the yellow fever virus vaccine, the chickenpox vaccine, the shingles vaccine, and one influenza virus vaccine. LAVs cause a real, but often asymptomatic infection in vaccine recipients, and hence usually elicit both humoral and cellular immune responses. In addition, an intranasally administered LAV will not only avoid needle sticks but may be more effective in eliciting immunity at the mucosal membrane. The latter is especially desirable for prevention of COVID-19 because the human upper respiratory airway tends to be less protected by existing vaccines that are administered intramuscularly. One obstacle to the development of LAVs against SARS-CoV-2 is the safety of the vaccine virus. Multi-layer attenuation of pathogenesis is expected to ensure a LAV does not revert to virulence.

### SUMMARY

Described herein are engineered SARS-CoV-2 variants having a combination of attenuating modifications and their use as live-attenuated vaccines (LAVs).

Provided herein are live-attenuated SARS-CoV-2 variants with a recombinant genome. The recombinant genome encodes a modified spike (S) protein with a deletion of the polybasic site (ΔPRRA), and encodes a modified non-structural protein 1 (Nsp1) with K164A and H165A substitutions. The recombinant genome also includes a mutation that prevents expression of open reading frames (ORFs) 6, 7a, 7b and 8 (such as a deletion of ORFs 6, 7a, 7b and 8). The disclosed live-attenuated SARS-CoV-2 retain the capacity to infect and replicate in mammalian cells. In some aspects, the live-attenuated SARS-CoV-2 is a Wuhan strain SARS-CoV-2, or a variant thereof from the Alpha, Beta, Delta, Gamma, Epsilon, Eta, Iota, Kappa, Mu, Zeta or Omicron lineage. In some examples, the variant is a variant of concern (VOC), such as a VOC from the Delta lineage or the Omicron lineage.

Also provided are immunogenic compositions that include a live-attenuated SARS-CoV-2 disclosed herein and a pharmaceutically acceptable carrier. In some aspects, the immunogenic composition further includes an adjuvant. In some aspects, the immunogenic composition is formulated for intranasal administration.

Further provided are nucleic acid molecules that include the complement of the recombinant genome of the attenuated SARS-CoV-2.

Also provided are collections of reverse genetics plasmids that include the complement of the recombinant SARS-CoV-2 genome. A method of producing a live-attenuated SARS-CoV-2 that includes transfecting permissive cells with the collection of reverse genetics plasmids; culturing the transfected cells under conditions sufficient to allow for replication of the attenuated SARS-CoV-2; and isolating the attenuated SARS-CoV-2 from the cell culture is further provided. Attenuated SARS-CoV-2 produced by the disclosed method is also provided.

Further provided are methods of eliciting an immune response against SARS-CoV-2 in a subject. In some aspects, the method includes administering to the subject an effective amount of a live-attenuated SARS-CoV-2 or immunogenic composition disclosed herein. In some examples, the live-attenuated SARS-CoV-2 or the immunogenic composition is administered intranasally. The live-attenuated SARS-CoV-2 or the immunogenic composition can also be used as part of a prime-boost immunization protocol. In some examples, the live-attenuated SARS-CoV-2 or the immunogenic composition is used as both the prime and boost. In other examples, the live-attenuated SARS-CoV-2 or the immunogenic composition is used in combination with a second SARS-CoV-2 vaccine.

The foregoing and other features of this disclosure will become more apparent from the following detailed description of several aspects which proceeds with reference to the accompanying figures.

### BRIEF DESCRIPTION OF THE DRAWINGS

FIGS. 1A-1E: Rational attenuation of SARS-CoV-2 WA1/2020. (FIG. 1A) Top, genome organization of SARS-CoV-2. Leader sequence, transcriptional regulatory sequence within the leader sequence (TRS-L) and within the body (TRS-B) are indicated. Bottom, either the polybasic insert “PRRA” alone or together with ORF6-8 were removed from the WA1/2020 genome. Locations of K164A/H165A and N128S/K129E are indicated. (FIG. 1B) Representative images of plaques formed by individual recombinant virus in Vero E6 cells. (FIG. 1C) Sanger sequencing result (SEQ ID NO: 33) of Nsp1-K164A/11165A virus after passage 5. (FIG. 1D) A549-hACE2 cells were inoculated with indicated virus at a multiplicity of infection (MOI) of 0.01. Output virus in the supernatants were determined on VeroE6 cells by plaque assay. (FIG. 1E) MatTek EpiAirway cells were inoculated with indicated virus at MOI of 5. Supernatants collected at 1, 2, 3, 4, 5 days post-infection (DPI) were titrated using a TCID₅₀ assay.

FIGS. 2A-2W: Attenuation of Nsp1-K164A/H165A in the respiratory tract of K18-hACE2 mice. (FIG. 2A) Schematic of the study design. (FIGS. 2B-2D) Weight loss (FIG. 2B), survival (FIG. 2C) and clinical scores (FIG. 2D) were recorded in infected mice for up to 8 days following infection. (FIGS. 2E-2F) Infectious viral titers of nasal turbinates at 2 DPI (FIG. 2E) or 4 DPI (FIG. 2F) were determined by plaque forming assays. Each solid circle represents one animal. **p<0.01. ns, non-significant. (FIGS. 2G-21) Infectious viral titers of lung homogenates at 2 DPI (FIG. 2G), 4 DPI (FIG. 2H) and 6 DPI (FIG. 21). **p<0.01. (FIG. 2J) Log₁₀-transformed subgenomic RNA (sgRNA) titers (E gene) were quantified by RT-qPCR. *p<0.05. (FIGS. 2K-2M) Infectious viral titers of brain homogenates at 2 DPI (FIG. 2K), 4 DPI (FIG. 2L) and 6 DPI (FIG. 2M). *p<0.05, **p<0.01. (FIGS. 2N-2W) HE stained lungs from uninfected animals (FIGS. 2N, 2O), or animals infected with WA1/2020 (FIGS. 2P, 2Q), ΔPRRA (FIGS. 2R, 2S), Nsp1-K164A/H165A (FIGS. 2T, 2U), or Nsp1-N128S/K129E (FIGS. 2V, 2W). Dotted lines denote areas of impact (consolidated). FIGS. 2O, 2Q, 2S, 2U and 2W are closeup images of FIGS. 2N, 2P, 2R, 2T and 2V, respectively.

FIGS. 3A-3T: Attenuation of Nsp1-K164A/H165A in Syrian hamsters. (FIG. 3A) Weight loss of Syrian hamsters after infection with 10⁴ PFU of WA1/2020 (n=7), ΔPRRA (n=7), Nsp1-N128S/K129E (n=7), Nsp1-K164A/H165A (n=4), or PBS (n=11). (FIG. 3B) Infectious titers in nasal wash samples WA1/2020 (n=11), ΔPRRA (n=15), Nsp1-N128S/K129E (n=17), Nsp1-K164A/H165A (n=8), or PBS (n=2). Dotted lines and filled areas marked by dotted lines indicate error bands. The limit of quantification is 200 TCIDso/ml. (FIG. 3C) Log₁₀-transformed sgRNA (E gene) titers in the lung and nasal turbinates at 4 DPI. Each solid circle denotes one animal. *p<0.05. (FIG. 3D) Log₁₀-transformed infectious titers in the lung at 4 DPI. *p<0.05, **p<0.01, ****p<0.0001. (FIG. 3E) Cumulative histopathology scores of infected lungs at 4 DPI. ****p<0.0001. (FIGS. 3F-3J) Representative images from HE stained lungs from hamsters infected by PBS (FIG. 3F), WA1-2020 (FIG. 3G), ΔPRRA (FIG. 3H), Nsp1-K164A/H165A (FIG. 31), or Nsp1-N128S/K129E (FIG. 3J). Dotted lines denote areas of impact (consolidated). (FIGS. 3K-30) Representative images of bronchioles corresponding to FIGS. 3F-3J, respectively. The images show massive luminal immune infiltrates (FIG. 3L), peribronchiolar infiltrates (white circle; FIG. 3M), and peribronchiolar infiltrates (white circle; FIG. 30). (FIGS. 3P-3T) Representative images of alveolar space corresponding to FIGS. 3F-3J, respectively. (FIG. 3Q) Oedema, immune infiltrates, and alveolar wall thickening. (FIG. 3R) Alveolar wall thickening, loss of alveolar space, and type II pneumocyte hyperplasia. (FIG. 3T) Loss of alveolar space and alveolar wall thickening.

FIGS. 4A-4G: Attenuation of virus propagation, macrophage accumulation, and epithelial damage in Nsp1-K164A/H165A-infected hamster lungs. (FIGS. 4A-4E) Representative images of serial lung sections immunostained for Iba1 and prosurfactant protein C (ProSPC) or SARS-CoV-2 nucleocapsid protein after infection with PBS (FIG. 4A), WA1/2020 (FIG. 4B), ΔPRRA (FIG. 4C), Nsp1-K164A/H165A (FIG. 4D), or Nsp1-N128S/K129E (FIG. 4E). Consolidation regions in WA1/2020 infected lungs show massive Iba1 positive macrophage infiltration around affected bronchioles (FIG. 4B). Alveolar epithelium surrounding consolidated regions in WA1/2020 stained prominently for viral nucleocapsid (FIG. 4B), while nucleocapsid staining is limited to bronchiolar epithelium in Nsp1-K164A/H165A (inset, FIG. 4D). (FIG. 4F) Digitally magnified images of macrophage-rich consolidation regions in WA1/2020 infected lungs show loss of ProSPC-stained alveolar type 2 cells. Viral antigen staining of infiltrates within and surrounding affected bronchioles with loss of RAGE-expressing type 1 epithelium in the same regions with consolidated macrophages (asterisk, FIG. 4G). Nuclei were counterstained with Hoechst 33342 dye. Scale bars: 500 mm (FIGS. 4A-4E), 100 mm (FIGS. 4F-4G).

FIGS. 5A-5D: Heatmap analysis of interferon and inflammation signalling pathways in nasal turbinates. Genes associated with inflammation (FIG. 5A), interferon-alpha response (FIG. 5B), interferon-gamma response (FIG. 5C), and TLR responses (FIG. 5D) are presented. Each solid square represents one hamster. Five groups (uninfected, WA1/2020, ΔPRRA, Nsp1-K164A/H165A and Nsp1-N128S/K129E) were each coded in a different color. The data represent the Z-scores derived from FPKM values of RNAseq transcriptomic analysis. Positive Z-Score denotes upregulation, and negative Z-score denotes downregulation.

FIGS. 6A-6L: Intranasal immunization of Syrian hamsters with Nsp1-K164A/H165A induced potent humoral responses and protected against WA1/2020 challenge. (FIG. 6A) Schematic of the study design. (FIGS. 6B, 6C) Serum antibody titers at 0-, 14-, and 28-days post-immunization measured by RBD (spike) binding ELISA (FIG. 6B) or anti-spike neutralizing antibody titers at 28 DPI (FIG. 6C). ****p<0.0001. (FIG. 6D) Weight loss profiles of immunized and convalescent hamsters after re-challenge with WA1/2020. (FIGS. 6E-6H) Infectious viral titers detected in nasal wash samples collected at 1-, 2-, 3- and 4-days post-challenge (DPC). **p<0.01, ***p<0.001, ****p<0.0001. (FIGS. 6I-6J) Infectious viral titers (FIG. 6I) and sgRNA titers (FIG. 6J) from tissues at 4 DPC. (FIGS. 6K, 6L) Infectious viral titers and sgRNA titers from tissues at 7DPC, respectively. *p<0.05, **p<0.01, ***p<0.001, ****p<0.0001.

FIGS. 7A-7C: Syrian hamsters immunized with Nsp1-K164A/H165A displayed minimal lung pathology upon WA1/2020 challenge. This figure summarizes the results after histopathological examination of the hamsters from FIG. 6. (FIG. 7A) Percentages of impacted areas in the lungs at 4 and 7 DPC. (FIG. 7B) Cumulative histopathology scores in the lungs at 7 DPC. (FIG. 7C) Heatmap of histopathology scores in the lungs at 7 DPC based on each category (see Example 1 for scoring criteria). Each solid square on top of the heatmap represents one hamster. Seven groups are shown: (i) uninfected, (ii) mock vaccinated and then challenged, (iii) WA1/2020 infected and then challenged, (iv) ΔPRRA infected and then challenged, (v) Nsp1-N128S/K129E infected and challenged, (vi) immunized with 1000 PFU Nsp1-K164A/H165A and (vii) challenged, immunized with 100 PFU Nsp1-K164A/H165A and challenged.

FIG. 8: Presence of viral RNA in infected hamster lungs. Viral RNA was detected using a specific probe. Nuclei were stained by haematoxylin.

FIGS. 9A-9E: Attenuation of Nsp1-K164A111165A in trachea of Syrian hamsters. (FIG. 9A) Normal epithelium of trachea from PBS-treated hamsters. (FIG. 9B) WA1/2020 infected trachea had luminal neutrophilic accumulations (*) as well as submucosal lymphoplasmacytic and neutrophil infiltrates (**). (FIG. 9C) Submucosal lymphoplasmacytic infiltrates. (FIG. 9D) A few lymphocytes (***) were found in submucosal space. (FIG. 9E) Submucosal lymphoplasmacytic infiltrates.

FIG. 10: Bulk transcriptomic profiling of hamster lung tissues at day 4 post SARS-CoV-2 infection. Heatmap analysis of interferon and inflammation signalling pathways in nasal turbinates. The data represents the Z-scores derived from FPKM values of RNAseg transcriptomic analysis. Positive Z-Score indicates genes that are upregulated, and negative Z-score indicates genes that are downresulated.

FIG. 11: Syrian hamsters immunized with Nsp1-K164A/H165A displayed minimal lung pathology upon WA1/2020 challenge. Representative HE stained images of hamster lungs corresponding to FIG. 6 are presented. An image of multiple lobes as well as an image from the alveolar space from each group are included.

FIG. 12: Antibody response of immunized and convalescent hamsters following re-challenge. RBD binding antibody titers were determined by ELISA from serum samples collected from unvaccinated, immunized, and convalescent hamsters at 0, 4, and 7 DPC. At 4 and 7 DPC, RBD IgG titers increased in the unvaccinated group but not in the other groups.

FIGS. 13A-13H: Intranasal immunization with 100 PFU Nsp1-K164A/H165A induces IgG and IgA. (FIG. 13A) Genome organization of the attenuated SARS-CoV-2-ΔPRRA-ΔORF6-8-Nsp1^{K164A/H165A} (abbreviated “Nsp1-K164A/H165A”). The polybasic insert (PRRA) together with ORF6-8 were removed from the WA1/2020 genome. Locations of K164A/H165A within Nsp1 are indicated at the bottom left of the panel. (FIGS. 13B-13H) Hamster sera or nasal wash samples were collected at 14- and 30-days post-intranasal inoculation of 100 PFU Nsp1-K164A/H165A or WA1/2020 and then tested for binding to WA1-2020 receptor binding domain (RBD) by ELISA (FIG. 13B) and for neutralization against WA1/2020 (FIG. 13C). Samples from naïve hamsters were included as negative controls. (FIG. 13D) Secretory IgA levels in nasal wash samples (NW IgA) collected 30 DPI were measured by ELISA (n=14). (FIGS. 13E-13H) Hamster sera of 30 DPI were measured for anti-Delta RBD IgG (FIG. 13E) and neutralization (FIG. 13F), and for anti-Omicron BA.1 RBD IgG (FIG. 13G) and neutralization (FIG. 13H). Bar graphs indicate mean titers with standard deviations shown as error bars. Each solid circle indicates individual hamsters from a single experiment. Statistical differences were calculated using ordinary one-way analysis of variance (ANOVA) in GraphPad Prism 9.4.0 with Tukey's multiple comparisons tests. For statistical significance, * indicates p<0.05 and **** indicates p<0.0001.

FIGS. 14A-14F: Intranasal immunization with Nsp1-K164A/H165A induces mucosal and systemic humoral immunity and cellular immunity. Male (4-month-old, n=8) Syrian hamsters were intranasally vaccinated with 10′ PFU Nsp1-K164A/H165A. Animals were bled at 14 and 28 DPI to collect sera and n=4 animals were euthanized at each time point to collect broncho-alveolar lavage fluid (BALF). Anti-RBD IgG titers against WA1/2020 or BA.1 in serum (FIG. 14A) and BALF (FIG. 14B) were measured by ELISA. Anti-RBD IgA was likewise quantified in serum (FIG. 14C) and BALF (FIG. 14D). (FIG. 14E) Serum neutralizing antibody (nAb) titers at 28 DPI were measured against WA1/2020 (WA1), Delta (B.1.617.2), as well as Omicron subvariants (BA.1, BA.2.12.1, BA.4, BA.5) using a 50% focus reduction neutralization (FRNT₅₀) assay. (FIG. 14F) Splenocytes were isolated from naive and vaccinated hamsters at 14 DPI and pulsed with 10 M WA1/2020 spike (S) and nucleocapsid (N) antigen pools for 48 hours. IFNγ-secreting splenocytes were enumerated by ELISPOT. Bar graphs in FIGS. 14A-14E represent samples collected at two time points from the same animals in a single experiment with dots representing individual animals. Statistical differences were calculated using ordinary one-way analysis of variance (ANOVA) in GraphPad Prism 9.4.0 with Tukey's multiple comparisons tests. ELISPOT data were compared using two-way ANOVA with Sidak's multiple comparison test. For statistical significance, * indicates p<0.05, ** indicates p<0.01, and **** indicates p<0.0001.

FIGS. 15A-15F: Intranasal immunization of Syrian hamsters with 100 PFU Nsp1-K164A/H165A significantly reduces viral loads in both upper and lower respiratory tract following challenge with Delta and Omicron variants. (FIG. 15A) Syrian hamsters were vaccinated with 100 PFU Nsp1-K164A/H165A or infected with 100 PFU WA1/2020 35 days prior to challenge with 10⁴ PFU Delta (n=6) or BA.1 Omicron (n=7) isolates on day 0. (FIGS. 15B-15C) From 1-5 DPC, infectious virus from nasal wash samples was quantified by focus-forming assays (FFA) in vaccinated, convalescent, or unvaccinated hamsters (n=8 for Delta, FIG. 15B; n=7 for Omicron BA. 1, FIG. 15C). Graphs for FIG. 15B and FIG. 15C indicate mean values from a single experiment with standard deviations shown as error bars. (FIG. 15D) Viral sgRNA levels in lung, trachea, and nasal turbinate samples from 4 DPC (n=4 each, except WA1/2020-Delta at n=3) were measured by qRT-PCR. (FIG. 15E) Infectious virus titers of lung homogenates at 4 DPC were determined by FFA. (FIG. 15F) Viral sgRNA levels in lungs and trachea at 7 DPC with Delta and BA.1 Omicron were measured by qRT-PCR. Dot plots represent samples collected from individual animals in a single experiment. Statistical differences were calculated using ordinary one-way analysis of variance (ANOVA) in GraphPad Prism 9.4.0 with Tukey's multiple comparisons tests. For statistical significance, ** indicates p<0.01, *** indicates p<0.001, and **** indicates p<0.0001. A: Delta variant challenge. O: Omicron BA.1 challenge.

FIGS. 16A-16D: Nsp1-K164A/H165A vaccination blocks virus propagation and MX1 induction in hamster lungs. Syrian hamsters were vaccinated with a low (100 PFU) dose of Nsp1-K164A/H165A or WA1/2020 35 days prior to challenge with a Delta or BA.1 isolate on day 0. Serial lung sections from non-infected non-vaccinated hamsters (mock) or hamster at 4 DPC were stained by (FIG. 16A) H&E or double-immunostained for (FIG. 16B) SARS-CoV-2 nucleocapsid protein (NP) and MX1 (interferon-induced antiviral protein). In FIG. 16A, images are shown at one level of magnification (0.7×) while corresponding serial immunostained images in FIG. 16B are shown at two levels of magnification (0.7× and 5×) with white boxes delimiting the regions of magnification. (FIG. 16C) Semiquantitative analysis of viral NP staining in hamster lungs at 4 DPC. The plotted values represent the percent NP positive area as a function of the total lung area for each section (n=3-4 animals per group). (FIG. 16D) High magnification immunofluorescence/differential interference contrast images of NP and MX1 in representative bronchioles of lung sections from mock hamsters or Delta-infected non-vaccinated or Nsp1-K164A/H165A-vaccinated hamsters at 4 DPC. Prominent cytoplasmic and nuclear localization of MX1 was detected in NP-positive bronchiolar epithelium in Delta-infected unvaccinated hamsters compared to low cytoplasmic expression of MX1 in mock and vaccinated hamsters. Nuclei were counterstained with Hoechst 33342 dye. Scale bars: 5 mm (0.7×), 500 μm (5×), 20 m (60×). A: Delta variant challenge. O: Omicron BA.1 challenge.

FIGS. 17A-17G: Intranasal immunization of Syrian hamsters with Nsp1-K164A/H165A protects against Delta and Omicron challenge. (FIG. 17A) Weight change was recorded for hamsters (described in FIG. 15A) after challenge by Delta and Omicron BA.1 variants for 7 days. Percentage of consolidation (FIG. 17B) and pathology score (FIG. 17C) in fixed lung tissues were compared between WA1/2020 convalescent (n=7), Nsp1-K164A/H165A vaccinated (n=8), and unvaccinated control (n=8) lungs at 4 DPC. Individual pathologies were graded by severity and presented in a heat map (FIG. 17D). Percentage of consolidation (FIG. 17E) and pathology score (FIG. 17F) were also compared at 7 DPC between WA1/2020 convalescent (n=6), Nsp1-K164A/H165A vaccinated (n=6), and unvaccinated control (n=8) lungs. (FIG. 17G) Heat-map presentation of individual pathologies at 7 DPC. Dot plots represent samples collected from individual animals in a single experiment. Statistical differences were calculated using ordinary one-way analysis of variance (ANOVA) in GraphPad Prism 9.4.0 with Tukey's multiple comparisons tests. For statistical significance, * indicates p<0.05, *** indicates p<0.001, and **** indicates p<0.0001. A: Delta variant challenge. O: Omicron BA.1 challenge.

FIGS. 18A-18C: Nsp1-K164A/H165A vaccination protects against lung pathology post-challenge with Delta and Omicron isolates. Syrian hamsters were vaccinated with a low (100 PFU) dose of Nsp1-K164A/H165A or WA1/2020 35 days prior to challenge with Delta or BA.1 Omicron isolates on day 0. Serial lung sections from non-infected non-vaccinated hamsters (mock) or 7 DPC-infected hamsters were stained by (FIG. 18A) H&E or double-immunostained for either (FIG. 18B) Iba1 (macrophage marker) and prosurfactant protein C (ProSPC, AT2 marker) or (FIG. 18C) E-cadherin (ECAD, epithelial junctional marker) and RAGE (AT1 marker). Delta-infected unvaccinated lungs (n=4) show extensive areas of tissue consolidation (FIG. 18A) that correspond with regions showing abundant Iba1-labeled macrophage accumulation and loss of ProSPC-labeled AT2 cells (FIG. 18B) as well as loss of alveolar wall RAGE-expressing AT1 epithelium surrounding affected ECAD-stained bronchioles and aberrant reepithelization (FIG. 18C). Similar though less extensive pathology was observed in two of four BA. 1-challenged unvaccinated hamsters. Nsp1-K164A/H165A or WA1/2020 inoculation completely prevented or suppressed Delta or BA.1-induced lung pathology. In FIG. 18A, images are shown at one level of magnification (0.7×) while corresponding serial immunostained images in FIG. 18B and FIG. 18C are shown at three levels of magnification (0.7×, 10×, and 40×) with white boxes delimiting the regions of magnification. Nuclei were counterstained with Hoechst 33342 dye. Scale bars: 5 mm (0.7×), 250 m (10×), 100 m (40×).

FIGS. 19A-19H: Airborne transmission of Nsp1-K164A/H165A in Syrian hamsters. (FIG. 19A) Donor Syrian hamsters (male, 5-month-old) were first inoculated with 100 PFU Nsp1-K164A/H165A (n=14) or WA1/2020 (n=14). One day after inoculation, donor hamsters were paired with recipient hamsters (sentinel, n=7/group) in the specially designed cages with metal dividers for monitoring airborne transmission. During pairing, nasal swabs were collected daily from sentinel hamsters for 4 days. (FIG. 19B) Weight loss profile of donor hamsters after virus inoculation. Symbols in FIG. 19B indicate mean percent weight change data of groups of hamsters (n=14) relative to initial individual animal weights on day 0. Statistical differences were calculated in GraphPad Prism 9.4.0 using a 2-way analysis of variance (ANOVA) with Sidak's multiple comparisons test. (FIG. 19C) Infectious virus titers of nasal wash samples collected from donor hamsters were measured by a TCID₅₀ assay for up to 5 days post-inoculation. Symbols indicate individual hamsters from a single experiment (n=14 per group). Statistical differences were calculated by Student's unpaired t-test in GraphPad Prism 9.4.0. (FIG. 19D) Infectious virus titers of nasal wash samples collected from sentinel hamsters were measured by a TCID₅₉ assay for up to 4 days post-exposure (DPE) (n=7 per group). (FIG. 19E) Weight loss profile of sentinel hamsters (n=7 per group) up to 14 days post-exposure to donor hamsters. (FIG. 19F) Seroconversion of sentinel hamsters was confirmed by ELISA measuring anti-WA1/2020 RBD IgG. One hamster (indicated by a diamond) after exposure to Nsp1-K164A/H165A did not seroconvert. Serum nAb titers against WA1/2020 (FIG. 19G) and BA.2.12.1 (FIG. 19H) in sentinel hamsters (n=6 for sentinels exposed to Nsp1-K164A/H165A and N=7 for those exposed to WA/2020) after four and half months post-exposure (MPE) were measured by focus forming reduction neutralization assays.

FIGS. 20A-20I: Seroconverted sentinel hamsters (passively vaccinated through transmission of Nsp1-K164A/H165A) are protected from BA.2.12.1 challenge. (FIG. 20A) Seroconverted sentinel hamsters (4.5 months after exposure to WA1-2020 and Nsp1-K164A/H165A) were challenged with 10⁴ PFU of BA.2.12.1. A group of 8 age-matched naïve hamsters were also included in the challenge study as controls. Weight change was recorded for 7 days post-challenge. (FIGS. 20B-20D) Infectious virus titers from nasal swabs (FIG. 20B), BALF (FIG. 20C) and lung homogenates (FIG. 20D) were measured by FFA. (FIG. 20E) Viral sgRNA levels in the lungs were quantified by RT-qPCR 4 DPC and 7 DPC. Dot plots represent samples collected from individual animals in a single experiment. Percentage of consolidation (FIG. 20F) and pathology scores in lungs (FIG. 20G) of sentinel hamsters (n=6 for Nsp1-K164A/H165A, n=7 for WA1/2020, and n=8 for naïve controls) at 4 and 7 DPC with 10⁴ PFU of BA.2.12.1. Individual lung pathologies at 4 DPC (FIG. 20H) and 7 DPC (FIG. 20I) are presented in heat maps. Dot plots in this graph represent samples collected from individual animals in a single experiment. Statistical differences were calculated using ordinary one-way analysis of variance (ANOVA) in GraphPad Prism 9.4.0 with Tukey's multiple comparisons tests. For statistical significance, * indicates p<0.05, ** indicates p<0.01), *** indicates p<0.001, and **** indicates p<0.0001.

FIGS. 21A-21K: Intranasal immunization of Syrian hamsters with 100 PFU Nsp1-K164A/H165A is protective against Omicron BA.5 challenge. (FIG. 21A) Syrian hamsters (male, 5 months old) were vaccinated with 100 PFU Nsp1-K164A/H165A 60 days prior to challenge with 10⁴ PFU BA.5 (isolate hCoV-19/USA/COR-22-063113/2022) (n=5) on day 0. (FIG. 21B) Changes in weight were followed in challenged hamsters 0-7 DPC, with ** indicating p<0.01 and **** indicating p<0.0001. (FIG. 21C) from 1-5 DPC, infectious virus from nasal wash samples was quantified by FFA for vaccinated and unvaccinated hamsters (n=4). (FIGS. 21D-21F) Infectious virus titers of nasal turbinates (FIG. 21D), BALF (FIG. 21E), and lung homogenates (FIG. 21F) at 4 and 7 DPC were determined by FFA. (FIG. 21G) Viral sgRNA levels in lung and nasal turbinate samples from 4 DPC (n=4) were measured by qRT-PCR. Sum clinical scores (FIG. 21H) and percentage of consolidation (FIG. 21I) were also compared for lungs collected at 4 and 7 DPC. Heat-map presentation of individual pathologies in lungs collected at 4 DPC (FIG. 21J) and 7 DPC (FIG. 21K). Graphs for FIG. 21B and FIG. 21G indicate mean values from a single experiment with standard deviations shown as error bars. Dot plots represent samples collected from individual animals in a single experiment, horizontal bars indicate mean values with standard deviations shown as error bars. Statistical differences were calculated using ordinary one-way analysis of variance (ANOVA) in GraphPad Prism 9.4.0 with Tukey's multiple comparisons tests and p-values are indicated in the graph where appropriate (p<0.05).

FIGS. 22A-22D: BA.1-LAV and BA.5-LAV as boosters. (FIG. 22A) Genome organization of BA.1-LAV and BA.5-LAV. Leader sequence, transcriptional regulatory sequence within the leader sequence and within the body are indicated. The polybasic insert (HRRA) was removed from the spike proteins and ORF6-8 were removed from the WA1/2020 backbone. Locations of K164A/H165A are highlighted in the figure. (FIG. 22B) Pseudovirus (PsV) bearing BA.5 spike but not WA1/2020 spike infected 293-mACE2 cell line. Infected cells are shown by fluorescent staining. DAPI was used to stain nuclei. (FIG. 22C) Overall study design to test BA.1-LAV and BA.5-LAV as boosters. 10-week-old male Balb/c mice were employed in this study. (FIG. 22D) Neutralizing antibody titers were measured from pre- and post-boost mouse sera. Each solid circle represents one animal. Numbers above each group indicate the geometric means of neutralizing antibody titers.

FIGS. 23A-23C: Nsp1-K164A/H165A protects against TUNEL-positive cell death in hamster lungs post-challenge with Delta and Omicron isolates. Syrian hamsters were vaccinated with Nsp1-K164A/H165A or WA1/2020 35 days prior to challenge with Delta or BA.1 Omicron isolates on day 0. TUNEL reactivity was examined in lung sections from (FIG. 23A) non-infected non-vaccinated hamsters (mock) or following challenge with (FIG. 23B) Delta or (FIG. 23C) Omicron at 7 DPI (n=3 hamsters per group). Nuclei were counterstained with hematoxylin. Black boxes indicate the regions of magnification. Scale bars: 5 mm (0.7×), 250 mm (10×).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the complete genome sequence of the Wuhan strain of SARS-CoV-2 (SARS-CoV-2/human/USA/WA-CDC-WA1/2020, deposited under GENBANK™ Accession No. MN985325.1).

```
        1 attaaaggtt tataccttcc caggtaacaa accaaccaac tttcgatctc ttgtagatct
      61 gttctctaaa cgaactttaa aatctgtgtg gctgtcactc ggctgcatgc ttagtgcact
    121 cacgcagtat aattaataac taattactgt cgttgacagg acacgagtaa ctcgtctatc
    181 ttctgcaggc tgcttacggt ttcgtccgtg ttgcagccga tcatcagcac atctaggttt
    241 cgtccgggtg tgaccgaaag gtaagatgga gagccttgtc cctggtttca acgagaaaac
    301 acacgtccaa ctcagtttgc ctgttttaca ggttcgcgac gtgctcgtac gtggctttgg
    361 agactccgtg gaggaggtct tatcagaggc acgtcaacat cttaaagatg gcacttgtgg
    421 cttagtagaa gttgaaaaag gcgttttgcc tcaacttgaa cagccctatg tgttcatcaa
    481 acgttcggat gctcgaactg cacctcatgg tcatgttatg gttgagctgg tagcagaact
    541 cgaaggcatt cagtacggtc gtagtggtga gacacttggt gtccttgtcc ctcatgtggg
    601 cgaaatacca gtggcttacc gcaaggttct tcttcgtaag aacggtaata aaggagctgg
    661 tggccatagt tacggcgccg atctaaagtc atttgactta ggcgacgagc ttggcactga
    721 tccttatgaa gattttcaag aaaactggaa cactaaacat agcagtggtg ttacccgtga
    781 actcatgcgt gagcttaacg gaggggcata cactcgctat gtcgataaca acttctgtgg
    841 ccctgatggc taccctcttg agtgcattaa agaccttcta gcacgtgctg gtaaagcttc
    901 atgcactttg tccgaacaac tggactttat tgacactaag aggggtgtat actgctgccg
    961 tgaacatgag catgaaattg cttggtacac ggaacgttct gaaaagagct atgaattgca
  1021 gacacctttt gaaattaaat tggcaaagaa atttgacacc ttcaatgggg aatgtccaaa
  1081 ttttgtattt cccttaaatt ccataatcaa gactattcaa ccaagggttg aaaagaaaaa
  1141 gcttgatggc tttatgggta gaattcgatc tgtctatcca gttgcgtcac caaatgaatg
  1201 caaccaaatg tgcctttcaa ctctcatgaa gtgtgatcat tgtggtgaaa cttcatggca
  1261 gacgggcgat tttgttaaag ccacttgcga attttgtggc actgagaatt tgactaaaga
  1321 aggtgccact acttgtggtt acttacccca aaatgctgtt gttaaaattt attgtccagc
  1381 atgtcacaat tcagaagtag gacctgagca tagtcttgcc gaataccata atgaatctgg
  1441 cttgaaaacc attcttcgta agggtggtcg cactattgcc tttggaggct gtgtgttctc
  1501 ttatgttggt tgccataaca agtgtgccta ttgggttcca cgtgctagcg ctaacatagg
  1561 ttgtaaccat acaggtgttg ttggagaagg ttccgaaggt cttaatgaca accttcttga
  1621 aatactccaa aaagagaaag tcaacatcaa tattgttggt gactttaaac ttaatgaaga
  1681 gatcgccatt attttggcat ctttttctgc ttccacaagt gcttttgtgg aaactgtgaa
  1741 aggtttggat tataaagcat tcaaacaaat tgttgaatcc tgtggtaatt ttaaagttac
  1801 aaaaggaaaa gctaaaaaag gtgcctggaa tattggtgaa cagaaatcaa tactgagtcc
  1861 tctttatgca tttgcatcag aggctgctcg tgttgtacga tcaattttct cccgcactct
  1921 tgaaactgct caaaattctg tgcgtgtttt acagaaggcc gctataacaa tactagatgg
  1981 aatttcacag tattcactga gactcattga tgctatgatg ttcacatctg atttggctac
  2041 taacaatcta gttgtaatgg cctacattac aggtggtgtt gttcagttga cttcgcagtg
  2101 gctaactaac atctttggca ctgtttatga aaaactcaaa cccgtccttg attggcttga
  2161 agagaagttt aaggaaggtg tagagtttct tagagacggt tgggaaattg ttaaatttat
  2221 ctcaacctgt gcttgtgaaa ttgtcggtgg acaaattgtc acctgtgcaa aggaaattaa
  2281 ggagagtgtt cagacattct ttaagcttgt aaataaattt ttggctttgt gtgctgactc
  2341 tatcattatt ggtggagcta aacttaaagc cttgaattta ggtgaaacat ttgtcacgca
  2401 ctcaaaggga ttgtacagaa agtgtgttaa atccagagaa gaaactggcc tactcatgcc
  2461 tctaaaagcc ccaaaagaaa ttatcttctt agagggagaa acacttocca cagaagtgtt
  2521 aacagaggaa gttgtcttga aaactggtga tttacaacca ttagaacaac ctactagtga
  2581 agctgttgaa gctccattgg ttggtacacc agtttgtatt aacgggctta tgttgctcga
  2641 aatcaaagac acagaaaagt actgtgccct tgcacctaat atgatggtaa caaacaatac
  2701 cttcacactc aaaggcggtg caccaacaaa ggttactttt ggtgatgaca ctgtgataga
  2761 agtgcaaggt tacaagagtg tgaatatcac ttttgaactt gatgaaagga ttgataaagt
  2821 acttaatgag aagtgctctg cctatacagt tgaactcggt acagaagtaa atgagttcgc
  2881 ctgtgttgtg gcagatgctg tcataaaaac tttgcaacca gtatctgaat tacttacacc
  2941 actgggcatt gatttagatg agtggagtat ggctacatac tacttatttg atgagtctgg
  3001 tgagtttaaa ttggcttcac atatgtattg ttctttctac cctccagatg aggatgaaga
  3061 agaaggtgat tgtgaagaag aagagtttga gccatcaact caatatgagt atggtactga
  3121 agatgattac caaggtaaac ctttggaatt tggtgccact tctgctgctc ttcaacctga
  3181 agaagagcaa gaagaagatt ggttagatga tgatagtcaa caaactgttg gtcaacaaga
  3241 cggcagtgag gacaatcaga caactactat tcaaacaatt gttgaggttc aacctcaatt
  3301 agagatggaa cttacaccag ttgttcagac tattgaagtg aatagtttta gtggttattt
  3361 aaaacttact gacaatgtat acattaaaaa tgcagacatt gtggaagaag ctaaaaaggt
  3421 aaaaccaaca gtggttgtta atgcagccaa tgtttacctt aaacatggag gaggtgttgc
  3481 aggagcctta aataaggcta ctaacaatgc catgcaagtt gaatctgatg attacatagc
  3541 tactaatgga ccacttaaag tgggtggtag ttgtgtttta agcggacaca atcttgctaa
  3601 acactgtctt catgttgtcg gcccaaatgt taacaaaggt gaagacattc aacttcttaa
  3661 gagtgcttat gaaaatttta atcagcacga agttctactt gcaccattat tatcagctgg
  3721 tatttttggt gctgacccta tacattcttt aagagtttgt gtagatactg ttcgcacaaa
  3781 tgtctactta gctgtctttg ataaaaatct ctatgacaaa cttgtttcaa gctttttgga
  3841 aatgaagagt gaaaagcaag ttgaacaaaa gatcgctgag attcctaaag aggaagttaa
  3901 gccatttata actgaaagta aaccttcagt tgaacagaga aaacaagatg ataagaaaat
  3961 caaagcttgt gttgaagaag ttacaacaac tctggaagaa actaagttcc tcacagaaaa
  4021 cttgttactt tatattgaca ttaatggcaa tottcatcca gattctgcca ctcttgttag
  4081 tgacattgac atcactttct taaagaaaga tgctccatat atagtgggtg atgttgttca
  4141 agagggtgtt ttaactgctg tggttatacc tactaaaaag gctggtggca ctactgaaat
  4201 gctagcgaaa gctttgagaa aagtgccaac agacaattat ataaccactt acccgggtca
  4261 gggtttaaat ggttacactg tagaggaggc aaagacagtg cttaaaaagt gtaaaagtgc
  4321 cttttacatt ctaccatcta ttatctctaa tgagaagcaa gaaattcttg gaactgtttc
  4381 ttggaatttg cgagaaatgc ttgcacatgc agaagaaaca cgcaaattaa tgcctgtctg
  4441 tgtggaaact aaagccatag tttcaactat acagcgtaaa tataagggta ttaaaataca
  4501 agagggtgtg gttgattatg gtgctagatt ttacttttac accagtaaaa caactgtagc
  4561 gtcacttatc aacacactta acgatctaaa tgaaactctt gttacaatgc cacttggcta
  4621 tgtaacacat ggcttaaatt tggaagaagc tgctcggtat atgagatctc tcaaagtgcc
  4681 agctacagtt tctgtttctt cacctgatgc tgttacagcg tataatggtt atcttacttc
  4741 ttcttctaaa acacctgaag aacattttat tgaaaccatc tcacttgctg gttcctataa
  4801 agattggtcc tattctggac aatctacaca actaggtata gaatttctta agagaggtga
  4861 taaaagtgta tattacacta gtaatcctac cacattccac ctagatggtg aagttatcac
  4921 ctttgacaat cttaagacac ttctttcttt gagagaagtg aggactatta aggtgtttac
  4981 aacagtagac aacattaacc tccacacgca agttgtggac atgtcaatga catatggaca
  5041 acagtttggt ccaacttatt tggatggagc tgatgttact aaaataaaac ctcataattc
  5101 acatgaaggt aaaacatttt atgttttacc taatgatgac actctacgtg ttgaggcttt
  5161 tgagtactac cacacaactg atcctagttt tctgggtagg tacatgtcag cattaaatca
  5221 cactaaaaag tggaaatacc cacaagttaa tggtttaact tctattaaat gggcagataa
  5281 caactgttat cttgccactg cattgttaac actccaacaa atagagttga agtttaatcc
  5341 acctgctcta caagatgctt attacagagc aagggctggt gaagctgcta acttttgtgc
  5401 acttatctta gcctactgta ataagacagt aggtgagtta ggtgatgtta gagaaacaat
  5461 gagttacttg tttcaacatg ccaatttaga ttcttgcaaa agagtcttga acgtggtgtg
  5521 taaaacttgt ggacaacagc agacaaccct taagggtgta gaagctgtta tgtacatggg
  5581 cacactttct tatgaacaat ttaagaaagg tgttcagata ccttgtacgt gtggtaaaca
  5641 agctacaaaa tatctagtac aacaggagtc accttttgtt atgatgtcag caccacctgc
  5701 tcagtatgaa cttaagcatg gtacatttac ttgtgctagt gagtacactg gtaattacca
  5761 gtgtggtcac tataaacata taacttctaa agaaactttg tattgcatag acggtgcttt
  5821 acttacaaag tcctcagaat acaaaggtcc tattacggat gttttctaca aagaaaacag
  5881 ttacacaaca accataaaac cagttactta taaattggat ggtgttgttt gtacagaaat
  5941 tgaccctaag ttggacaatt attataagaa agacaattct tatttcacag agcaaccaat
  6001 tgatcttgta ccaaaccaac catatccaaa cgcaagcttc gataatttta agtttgtatg
  6061 tgataatatc aaatttgctg atgatttaaa ccagttaact ggttataaga aacctgcttc
  6121 aagagagctt aaagttacat ttttccctga cttaaatggt gatgtggtgg ctattgatta
  6181 taaacactac acaccctctt ttaagaaagg agctaaattg ttacataaac ctattgtttg
  6241 gcatgttaac aatgcaacta ataaagccac gtataaacca aatacctggt gtatacgttg
  6301 tctttggagc acaaaaccag ttgaaacatc aaattcgttt gatgtactga agtcagagga
  6361 cgcgcaggga atggataatc ttgcctgcga agatctaaaa ccagtctctg aagaagtagt
  6421 ggaaaatcct accatacaga aagacgttct tgagtgtaat gtgaaaacta ccgaagttgt
  6481 aggagacatt atacttaaac cagcaaataa tagtttaaaa attacagaag aggttggcca
  6541 cacagatcta atggctgctt atgtagacaa ttctagtctt actattaaga aacctaatga
  6601 attatctaga gtattaggtt tgaaaaccct tgctactcat ggtttagctg ctgttaatag
  6661 tgtcccttgg gatactatag ctaattatgc taagcctttt cttaacaaag ttgttagtac
  6721 aactactaac atagttacac ggtgtttaaa ccgtgtttgt actaattata tgccttattt
  6781 ctttacttta ttgctacaat tgtgtacttt tactagaagt acaaattcta gaattaaagc
  6841 atctatgccg actactatag caaagaatac tgttaagagt gtcggtaaat tttgtctaga
  6901 ggcttcattt aattatttga agtcacctaa tttttctaaa ctgataaata ttataatttg
  6961 gtttttacta ttaagtgttt gcctaggttc tttaatctac tcaaccgctg ctttaggtgt
  7021 tttaatgtct aatttaggca tgccttctta ctgtactggt tacagagaag gctatttgaa
  7081 ctctactaat gtcactattg caacctactg tactggttct ataccttgta gtgtttgtct
  7141 tagtggttta gattctttag acacctatcc ttctttagaa actatacaaa ttaccatttc
  7201 atcttttaaa tgggatttaa ctgcttttgg cttagttgca gagtggtttt tggcatatat
  7261 tcttttcact aggtttttct atgtacttgg attggctgca atcatgcaat tgtttttcag
  7321 ctattttgca gtacatttta ttagtaattc ttggcttatg tggttaataa ttaatcttgt
  7381 acaaatggcc ccgatttcag ctatggttag aatgtacatc ttctttgcat cattttatta
  7441 tgtatggaaa agttatgtgc atgttgtaga cggttgtaat tcatcaactt gtatgatgtg
  7501 ttacaaacgt aatagagcaa caagagtcga atgtacaact attgttaatg gtgttagaag
  7561 gtccttttat gtctatgcta atggaggtaa aggcttttgc aaactacaca attggaattg
  7621 tgttaattgt gatacattct gtgctggtag tacatttatt agtgatgaag ttgcgagaga
  7681 cttgtcacta cagtttaaaa gaccaataaa tcctactgac cagtcttctt acatcgttga
  7741 tagtgttaca gtgaagaatg gttccatcca totttacttt gataaagctg gtcaaaagac
  7801 ttatgaaaga cattctctct ctcattttgt taacttagac aacctgagag ctaataacac
  7861 taaaggttca ttgcctatta atgttatagt ttttgatggt aaatcaaaat gtgaagaatc
  7921 atctgcaaaa tcagcgtctg tttactacag tcagcttatg tgtcaaccta tactgttact
  7981 agatcaggca ttagtgtctg atgttggtga tagtgcggaa gttgcagtta aaatgtttga
  8041 tgcttacgtt aatacgtttt catcaacttt taacgtacca atggaaaaac tcaaaacact
  8101 agttgcaact gcagaagctg aacttgcaaa gaatgtgtcc ttagacaatg tcttatctac
  8161 ttttatttca gcagctcggc aagggtttgt tgattcagat gtagaaacta aagatgttgt
  8221 tgaatgtctt aaattgtcac atcaatctga catagaagtt actggcgata gttgtaataa
  8281 ctatatgctc acctataaca aagttgaaaa catgacaccc cgtgaccttg gtgcttgtat
  8341 tgactgtagt gogcgtcata ttaatgcgca ggtagcaaaa agtcacaaca ttgctttgat
  8401 atggaacgtt aaagatttca tgtcattgtc tgaacaacta cgaaaacaaa tacgtagtgc
  8461 tgctaaaaag aataacttac cttttaagtt gacatgtgca actactagac aagttgttaa
  8521 tgttgtaaca acaaagatag cacttaaggg tggtaaaatt gttaataatt ggttgaagca
  8581 gttaattaaa gttacacttg tgttcctttt tgttgctgct attttctatt taataacacc
  8641 tgttcatgtc atgtctaaac atactgactt ttcaagtgaa atcataggat acaaggctat
  8701 tgatggtggt gtcactcgtg acatagcatc tacagatact tgttttgcta acaaacatgc
  8761 tgattttgac acatggttta gtcagcgtgg tggtagttat actaatgaca aagcttgccc
  8821 attgattgct gcagtcataa caagagaagt gggttttgtc gtgcctggtt tgcctggcac
  8881 gatattacgc acaactaatg gtgacttttt gcatttctta cctagagttt ttagtgcagt
  8941 tggtaacatc tgttacacac catcaaaact tatagagtac actgactttg caacatcagc
  9001 ttgtgttttg gctgctgaat gtacaatttt taaagatgct tctggtaagc cagtaccata
  9061 ttgttatgat accaatgtac tagaaggttc tgttgcttat gaaagtttac gccctgacac
  9121 acgttatgtg ctcatggatg gctctattat tcaatttcct aacacctacc ttgaaggttc
  9181 tgttagagtg gtaacaactt ttgattctga gtactgtagg cacggcactt gtgaaagatc
  9241 agaagctggt gtttgtgtat ctactagtgg tagatgggta cttaacaatg attattacag
  9301 atctttacca ggagttttct gtggtgtaga tgctgtaaat ttacttacta atatgtttac
  9361 accactaatt caacctattg gtgctttgga catatcagca tctatagtag ctggtggtat
  9421 tgtagctatc gtagtaacat gccttgccta ctattttatg aggtttagaa gagcttttgg
  9481 tgaatacagt catgtagttg cctttaatac tttactattc cttatgtcat tcactgtact
  9541 ctgtttaaca ccagtttact cattcttacc tggtgtttat tctgttattt acttgtactt
  9601 gacattttat cttactaatg atgtttcttt tttagcacat attcagtgga tggttatgtt
  9661 cacaccttta gtacctttct ggataacaat tgcttatatc atttgtattt ccacaaagca
  9721 tttctattgg ttctttagta attacctaaa gagacgtgta gtctttaatg gtgtttcctt
  9781 tagtactttt gaagaagctg cgctgtgcac ctttttgtta aataaagaaa tgtatctaaa
  9841 gttgcgtagt gatgtgctat tacctottac gcaatataat agatacttag ctctttataa
  9901 taagtacaag tattttagtg gagcaatgga tacaactagc tacagagaag ctgcttgttg
  9961 tcatctcgca aaggctctca atgacttcag taactcaggt tctgatgttc tttaccaacc
10021 accacaaacc tctatcacct cagctgtttt gcagagtggt tttagaaaaa tggcattccc
10081 atctggtaaa gttgagggtt gtatggtaca agtaacttgt ggtacaacta cacttaacgg
10141 tctttggctt gatgacgtag tttactgtcc aagacatgtg atctgcacct ctgaagacat
10201 gcttaaccct aattatgaag atttactcat tcgtaagtct aatcataatt tcttggtaca
10261 ggctggtaat gttcaactca gggttattgg acattctatg caaaattgtg tacttaagct
10321 taaggttgat acagccaatc ctaagacacc taagtataag tttgttcgca ttcaaccagg
10381 acagactttt tcagtgttag cttgttacaa tggttcacca tctggtgttt accaatgtgc
10441 tatgaggccc aatttcacta ttaagggttc attccttaat ggttcatgtg gtagtgttgg
10501 ttttaacata gattatgact gtgtctcttt ttgttacatg caccatatgg aattaccaac
10561 tggagttcat gctggcacag acttagaagg taacttttat ggaccttttg ttgacaggca
10621 aacagcacaa gcagctggta cggacacaac tattacagtt aatgttttag cttggttgta
10681 cgctgctgtt ataaatggag acaggtggtt tctcaatcga tttaccacaa ctcttaatga
10741 ctttaacctt gtggctatga agtacaatta tgaacctcta acacaagacc atgttgacat
10801 actaggacct ctttctgctc aaactggaat tgccgtttta gatatgtgtg cttcattaaa
10861 agaattactg caaaatggta tgaatggacg taccatattg ggtagtgctt tattagaaga
10921 tgaatttaca ccttttgatg ttgttagaca atgctcaggt gttactttcc aaagtgcagt
10981 gaaaagaaca atcaagggta cacaccactg gttgttactc acaattttga cttcactttt
11041 agttttagtc cagagtactc aatggtcttt gttctttttt ttgtatgaaa atgccttttt
11101 accttttgct atgggtatta ttgctatgtc tgcttttgca atgatgtttg tcaaacataa
11161 gcatgcattt ctctgtttgt ttttgttacc ttctcttgcc actgtagctt attttaatat
11221 ggtctatatg cctgctagtt gggtgatgcg tattatgaca tggttggata tggttgatac
11281 tagtttgtct ggttttaagc taaaagactg tgttatgtat gcatcagctg tagtgttact
11341 aatccttatg acagcaagaa ctgtgtatga tgatggtgct aggagagtgt ggacacttat
11401 gaatgtcttg acactcgttt ataaagttta ttatggtaat gctttagatc aagccatttc
11461 catgtgggct cttataatct ctgttacttc taactactca ggtgtagtta caactgtcat
11521 gtttttggcc agaggtattg tttttatgtg tgttgagtat tgccctattt tottcataac
11581 tggtaataca cttcagtgta taatgctagt ttattgtttc ttaggctatt tttgtacttg
11641 ttactttggc ctcttttgtt tactcaaccg ctactttaga ctgactcttg gtgtttatga
11701 ttacttagtt tctacacagg agtttagata tatgaattca cagggactac tcccacccaa
11761 gaatagcata gatgccttca aactcaacat taaattgttg ggtgttggtg gcaaaccttg
11821 tatcaaagta gccactgtac agtctaaaat gtcagatgta aagtgcacat cagtagtctt
11881 actctcagtt ttgcaacaac tcagagtaga atcatcatct aaattgtggg ctcaatgtgt
11941 ccagttacac aatgacattc tcttagctaa agatactact gaagcctttg aaaaaatggt
12001 ttcactactt tctgttttgc tttccatgca gggtgctgta gacataaaca agctttgtga
12061 agaaatgctg gacaacaggg caaccttaca agctatagcc tcagagttta gttcccttcc
12121 atcatatgca gcttttgcta ctgctcaaga agcttatgag caggctgttg ctaatggtga
12181 ttctgaagtt gttcttaaaa agttgaagaa gtctttgaat gtggctaaat ctgaatttga
12241 ccgtgatgca gccatgcaac gtaagttgga aaagatggct gatcaagcta tgacccaaat
12301 gtataaacag gctagatctg aggacaagag ggcaaaagtt actagtgcta tgcagacaat
12361 gcttttcact atgcttagaa agttggataa tgatgcactc aacaacatta tcaacaatgc
12421 aagagatggt tgtgttccct tgaacataat acctcttaca acagcagcca aactaatggt
12481 tgtcatacca gactataaca catataaaaa tacgtgtgat ggtacaacat ttacttatgc
12541 atcagcattg tgggaaatcc aacaggttgt agatgcagat agtaaaattg ttcaacttag
12601 tgaaattagt atggacaatt cacctaattt agcatggcct cttattgtaa cagctttaag
12661 ggccaattct gctgtcaaat tacagaataa tgagcttagt cctgttgcac tacgacagat
12721 gtcttgtgct gccggtacta cacaaactgc ttgcactgat gacaatgcgt tagcttacta
12781 caacacaaca aagggaggta ggtttgtact tgcactgtta tccgatttac aggatttgaa
12841 atgggctaga ttccctaaga gtgatggaac tggtactatc tatacagaac tggaaccacc
12901 ttgtaggttt gttacagaca cacctaaagg tcctaaagtg aagtatttat actttattaa
12961 aggattaaac aacctaaata gaggtatggt acttggtagt ttagctgcca cagtacgtct
13021 acaagctggt aatgcaacag aagtgcctgc caattcaact gtattatott tctgtgcttt
13081 tgctgtagat gctgctaaag cttacaaaga ttatctagct agtgggggac aaccaatcac
13141 taattgtgtt aagatgttgt gtacacacac tggtactggt caggcaataa cagttacacc
13201 ggaagccaat atggatcaag aatcctttgg tggtgcatcg tgttgtctgt actgccgttg
13261 ccacatagat catccaaatc ctaaaggatt ttgtgactta aaaggtaagt atgtacaaat
13321 acctacaact tgtgctaatg accctgtggg ttttacactt aaaaacacag tctgtaccgt
13381 ctgcggtatg tggaaaggtt atggctgtag ttgtgatcaa ctccgcgaac ccatgcttca
13441 gtcagctgat gcacaatcgt ttttaaacgg gtttgcggtg taagtgcagc ccgtcttaca
13501 ccgtgcggca caggcactag tactgatgtc gtatacaggg cttttgacat ctacaatgat
13561 aaagtagctg gttttgctaa attcctaaaa actaattgtt gtcgcttcca agaaaaggac
13621 gaagatgaca atttaattga ttcttacttt gtagttaaga gacacacttt ctctaactac
13681 caacatgaag aaacaattta taatttactt aaggattgtc cagctgttgc taaacatgac
13741 ttctttaagt ttagaataga cggtgacatg gtaccacata tatcacgtca acgtcttact
13801 aaatacacaa tggcagacct cgtctatgct ttaaggcatt ttgatgaagg taattgtgac
13861 acattaaaag aaatacttgt cacatacaat tgttgtgatg atgattattt caataaaaag
13921 gactggtatg attttgtaga aaacccagat atattacgcg tatacgccaa cttaggtgaa
13981 cgtgtacgcc aagctttgtt aaaaacagta caattctgtg atgccatgcg aaatgctggt
14041 attgttggtg tactgacatt agataatcaa gatctcaatg gtaactggta tgatttcggt
14101 gatttcatac aaaccacgcc aggtagtgga gttcctgttg tagattctta ttattcattg
14161 ttaatgccta tattaacctt gaccagggct ttaactgcag agtcacatgt tgacactgac
14221 ttaacaaagc cttacattaa gtgggatttg ttaaaatatg acttcacgga agagaggtta
14281 aaactctttg accgttattt taaatattgg gatcagacat accacccaaa ttgtgttaac
14341 tgtttggatg acagatgcat tctgcattgt gcaaacttta atgttttatt ctctacagtg
14401 ttcccaccta caagttttgg accactagtg agaaaaatat ttgttgatgg tgttccattt
14461 gtagtttcaa ctggatacca cttcagagag ctaggtgttg tacataatca ggatgtaaac
14521 ttacatagct ctagacttag ttttaaggaa ttacttgtgt atgctgctga ccctgctatg
14581 cacgctgctt ctggtaatct attactagat aaacgcacta cgtgcttttc agtagctgca
14641 cttactaaca atgttgcttt tcaaactgtc aaacccggta attttaacaa agacttctat
14701 gactttgctg tgtctaaggg tttctttaag gaaggaagtt ctgttgaatt aaaacacttc
14761 ttctttgctc aggatggtaa tgctgctatc agcgattatg actactatcg ttataatcta
14821 ccaacaatgt gtgatatcag acaactacta tttgtagttg aagttgttga taagtacttt
14881 gattgttacg atggtggctg tattaatgct aaccaagtca tcgtcaacaa cctagacaaa
14941 tcagctggtt ttccatttaa taaatggggt aaggctagac tttattatga ttcaatgagt
15001 tatgaggatc aagatgcact tttcgcatat acaaaacgta atgtcatccc tactataact
15061 caaatgaatc ttaagtatgc cattagtgca aagaatagag ctcgcaccgt agctggtgtc
15121 tctatctgta gtactatgac caatagacag tttcatcaaa aattattgaa atcaatagcc
15181 gccactagag gagctactgt agtaattgga acaagcaaat tctatggtgg ttggcacaac
15241 atgttaaaaa ctgtttatag tgatgtagaa aaccctcacc ttatgggttg ggattatcct
15301 aaatgtgata gagccatgcc taacatgctt agaattatgg cctcacttgt tcttgctcgc
15361 aaacatacaa cgtgttgtag cttgtcacac cgtttctata gattagctaa tgagtgtgct
15421 caagtattga gtgaaatggt catgtgtggc ggttcactat atgttaaacc aggtggaacc
15481 tcatcaggag atgccacaac tgcttatgct aatagtgttt ttaacatttg tcaagctgtc
15541 acggccaatg ttaatgcact tttatctact gatggtaaca aaattgccga taagtatgtc
15601 cgcaatttac aacacagact ttatgagtgt ctctatagaa atagagatgt tgacacagac
15661 tttgtgaatg agttttacgc atatttgcgt aaacatttct caatgatgat actctctgac
15721 gatgctgttg tgtgtttcaa tagcacttat gcatctcaag gtctagtggc tagcataaag
15781 aactttaagt cagttcttta ttatcaaaac aatgttttta tgtctgaagc aaaatgttgg
15841 actgagactg accttactaa aggacctcat gaattttgct ctcaacatac aatgctagtt
15901 aaacagggtg atgattatgt gtaccttcct tacccagatc catcaagaat cctaggggcc
15961 ggctgttttg tagatgatat cgtaaaaaca gatggtacac ttatgattga acggttcgtg
16021 tctttagcta tagatgctta cccacttact aaacatccta atcaggagta tgctgatgtc
16081 tttcatttgt acttacaata cataagaaag ctacatgatg agttaacagg acacatgtta
16141 gacatgtatt ctgttatgct tactaatgat aacacttcaa ggtattggga acctgagttt
16201 tatgaggcta tgtacacacc gcatacagtc ttacaggctg ttggggcttg tgttctttgc
16261 aattcacaga cttcattaag atgtggtgct tgcatacgta gaccattctt atgttgtaaa
16321 tgctgttacg accatgtcat atcaacatca cataaattag tcttgtctgt taatccgtat
16381 gtttgcaatg ctccaggttg tgatgtcaca gatgtgactc aactttactt aggaggtatg
16441 agctattatt gtaaatcaca taaaccaccc attagttttc cattgtgtgc taatggacaa
16501 gtttttggtt tatataaaaa tacatgtgtt ggtagcgata atgttactga ctttaatgca
16561 attgcaacat gtgactggac aaatgctggt gattacattt tagctaacac ctgtactgaa
16621 agactcaagc tttttgcagc agaaacgctc aaagctactg aggagacatt taaactgtct
16681 tatggtattg ctactgtacg tgaagtgctg tctgacagag aattacatct ttcatgggaa
16741 gttggtaaac ctagaccacc acttaaccga aattatgtct ttactggtta tcgtgtaact
16801 aaaaacagta aagtacaaat aggagagtac acctttgaaa aaggtgacta tggtgatgct
16861 gttgtttacc gaggtacaac aacttacaaa ttaaatgttg gtgattattt tgtgctgaca
16921 tcacatacag taatgccatt aagtgcacct acactagtgc cacaagagca ctatgttaga
16981 attactggct tatacccaac actcaatatc tcagatgagt tttctagcaa tgttgcaaat
17041 tatcaaaagg ttggtatgca aaagtattct acactccagg gaccacctgg tactggtaag
17101 agtcattttg ctattggcct agctctctac tacccttctg ctcgcatagt gtatacagct
17161 tgctctcatg ccgctgttga tgcactatgt gagaaggcat taaaatattt gcctatagat
17221 aaatgtagta gaattatacc tgcacgtgct cgtgtagagt gttttgataa attcaaagtg
17281 aattcaacat tagaacagta tgtcttttgt actgtaaatg cattgcctga gacgacagca
17341 gatatagttg tctttgatga aatttcaatg gccacaaatt atgatttgag tgttgtcaat
17401 gccagattac gtgctaagca ctatgtgtac attggcgacc ctgctcaatt acctgcacca
17461 cgcacattgc taactaaggg cacactagaa ccagaatatt tcaattcagt gtgtagactt
17521 atgaaaacta taggtccaga catgttcctc ggaacttgtc ggcgttgtcc tgctgaaatt
17581 gttgacactg tgagtgcttt ggtttatgat aataagctta aagcacataa agacaaatca
17641 gctcaatgct ttaaaatgtt ttataagggt gttatcacgc atgatgtttc atctgcaatt
17701 aacaggccac aaataggcgt ggtaagagaa ttccttacac gtaaccctgc ttggagaaaa
17761 gctgtcttta tttcacctta taattcacag aatgctgtag cctcaaagat tttgggacta
17821 ccaactcaaa ctgttgattc atcacagggc tcagaatatg actatgtcat attcactcaa
17881 accactgaaa cagctcactc ttgtaatgta aacagattta atgttgctat taccagagca
17941 aaagtaggca tactttgcat aatgtctgat agagaccttt atgacaagtt gcaatttaca
18001 agtcttgaaa ttccacgtag gaatgtggca actttacaag ctgaaaatgt aacaggactt
18061 tttaaagatt gtagtaaggt aatcactggg ttacatccta cacaggcacc tacacacctc
18121 agtgttgaca ctaaattcaa aactgaaggt ttatgtgttg acatacctgg catacctaag
18181 gacatgacct atagaagact catctctatg atgggtttta aaatgaatta tcaagttaat
18241 ggttacccta acatgtttat cacccgcgaa gaagctataa gacatgtacg tgcatggatt
18301 ggcttcgatg tcgaggggtg tcatgctact agagaagctg ttggtaccaa tttaccttta
18361 cagctaggtt tttctacagg tgttaaccta gttgctgtac ctacaggtta tgttgataca
18421 cctaataata cagatttttc cagagttagt gctaaaccac cgcctggaga tcaatttaaa
18481 cacctcatac cacttatgta caaaggactt ccttggaatg tagtgcgtat aaagattgta
18541 caaatgttaa gtgacacact taaaaatctc tctgacagag tcgtatttgt cttatgggca
18601 catggctttg agttgacatc tatgaagtat tttgtgaaaa taggacctga gcgcacctgt
18661 tgtctatgtg atagacgtgc cacatgcttt tccactgctt cagacactta tgcctgttgg
18721 catcattcta ttggatttga ttacgtctat aatccgttta tgattgatgt tcaacaatgg
18781 ggttttacag gtaacctaca aagcaaccat gatctgtatt gtcaagtcca tggtaatgca
18841 catgtagcta gttgtgatgc aatcatgact aggtgtctag ctgtccacga gtgctttgtt
18901 aagcgtgttg actggactat tgaatatcct ataattggtg atgaactgaa gattaatgcg
18961 gcttgtagaa aggttcaaca catggttgtt aaagctgcat tattagcaga caaattccca
19021 gttcttcacg acattggtaa ccctaaagct attaagtgtg tacctcaagc tgatgtagaa
19081 tggaagttct atgatgcaca gccttgtagt gacaaagctt ataaaataga agaattattc
19141 tattcttatg ccacacattc tgacaaattc acagatggtg tatgcctatt ttggaattgc
19201 aatgtcgata gatatcctgc taattccatt gtttgtagat ttgacactag agtgctatct
19261 aaccttaact tgcctggttg tgatggtggc agtttgtatg taaataaaca tgcattccac
19321 acaccagctt ttgataaaag tgcttttgtt aatttaaaac aattaccatt tttctattac
19381 tctgacagtc catgtgagtc tcatggaaaa caagtagtgt cagatataga ttatgtacca
19441 ctaaagtctg ctacgtgtat aacacgttgc aatttaggtg gtgctgtctg tagacatcat
19501 gctaatgagt acagattgta tctcgatgct tataacatga tgatctcagc tggctttagc
19561 ttgtgggttt acaaacaatt tgatacttat aacctctgga acacttttac aagacttcag
19621 agtttagaaa atgtggcttt taatgttgta aataagggac actttgatgg acaacagggt
19681 gaagtaccag tttctatcat taataacact gtttacacaa aagttgatgg tgttgatgta
19741 gaattgtttg aaaataaaac aacattacct gttaatgtag catttgagct ttgggctaag
19801 cgcaacatta aaccagtacc agaggtgaaa atactcaata atttgggtgt ggacattgct
19861 gctaatactg tgatctggga ctacaaaaga gatgctccag cacatatatc tactattggt
19921 gtttgttcta tgactgacat agccaagaaa ccaactgaaa cgatttgtgc accactcact
19981 gtcttttttg atggtagagt tgatggtcaa gtagacttat ttagaaatgc ccgtaatggt
20041 gttcttatta cagaaggtag tgttaaaggt ttacaaccat ctgtaggtcc caaacaagct
20101 agtcttaatg gagtcacatt aattggagaa gccgtaaaaa cacagttcaa ttattataag
20161 aaagttgatg gtgttgtcca acaattacct gaaacttact ttactcagag tagaaattta
20221 caagaattta aacccaggag tcaaatggaa attgatttct tagaattagc tatggatgaa
20281 ttcattgaac ggtataaatt agaaggctat gccttcgaac atatcgttta tggagatttt
20341 agtcatagtc agttaggtgg tttacatcta ctgattggac tagctaaacg ttttaaggaa
20401 tcaccttttg aattagaaga ttttattcct atggacagta cagttaaaaa ctatttcata
20461 acagatgcgc aaacaggttc atctaagtgt gtgtgttctg ttattgattt attacttgat
20521 gattttgttg aaataataaa atcccaagat ttatctgtag tttctaaggt tgtcaaagtg
20581 actattgact atacagaaat ttcatttatg ctttggtgta aagatggcca tgtagaaaca
20641 ttttacccaa aattacaatc tagtcaagcg tggcaaccgg gtgttgctat gcctaatctt
20701 tacaaaatgc aaagaatgct attagaaaag tgtgaccttc aaaattatgg tgatagtgca
20761 acattaccta aaggcataat gatgaatgtc gcaaaatata ctcaactgtg tcaatattta
20821 aacacattaa cattagctgt accctataat atgagagtta tacattttgg tgctggttct
20881 gataaaggag ttgcaccagg tacagctgtt ttaagacagt ggttgcctac gggtacgctg
20941 cttgtcgatt cagatcttaa tgactttgtc tctgatgcag attcaacttt gattggtgat
21001 tgtgcaactg tacatacagc taataaatgg gatctcatta ttagtgatat gtacgaccct
21061 aagactaaaa atgttacaaa agaaaatgac tctaaagagg gttttttcac ttacatttgt
21121 gggtttatac aacaaaagct agctcttgga ggttccgtgg ctataaagat aacagaacat
21181 tcttggaatg ctgatcttta taagctcatg ggacacttcg catggtggac agcctttgtt
21241 actaatgtga atgcgtcatc atctgaagca tttttaattg gatgtaatta tcttggcaaa
21301 ccacgcgaac aaatagatgg ttatgtcatg catgcaaatt acatattttg gaggaataca
21361 aatccaattc agttgtcttc ctattcttta tttgacatga gtaaatttcc ccttaaatta
21421 aggggtactg ctgttatgtc tttaaaagaa ggtcaaatca atgatatgat tttatctctt
21481 cttagtaaag gtagacttat aattagagaa aacaacagag ttgttatttc tagtgatgtt
21541 cttgttaaca actaaacgaa caatgtttgt ttttcttgtt ttattgccac tagtctctag
21601 tcagtgtgtt aatcttacaa ccagaactca attaccccct gcatacacta attctttcac
21661 acgtggtgtt tattaccctg acaaagtttt cagatcctca gttttacatt caactcagga
21721 cttgttctta cctttctttt ccaatgttac ttggttccat gctatacatg tctctgggac
21781 caatggtact aagaggtttg ataaccctgt cctaccattt aatgatggtg tttattttgc
21841 ttccactgag aagtctaaca taataagagg ctggattttt ggtactactt tagattcgaa
21901 gacccagtcc ctacttattg ttaataacgc tactaatgtt gttattaaag tctgtgaatt
21961 tcaattttgt aatgatccat ttttgggtgt ttattaccac aaaaacaaca aaagttggat
22021 ggaaagtgag ttcagagttt attctagtgc gaataattgc acttttgaat atgtctctca
22081 gccttttctt atggaccttg aaggaaaaca gggtaatttc aaaaatctta gggaatttgt
22141 gtttaagaat attgatggtt attttaaaat atattctaag cacacgccta ttaatttagt
22201 gcgtgatctc cctcagggtt tttcggcttt agaaccattg gtagatttgc caataggtat
22261 taacatcact aggtttcaaa ctttacttgc tttacataga agttatttga ctcctggtga
22321 ttcttcttca ggttggacag ctggtgctgc agcttattat gtgggttatc ttcaacctag
22381 gacttttcta ttaaaatata atgaaaatgg aaccattaca gatgctgtag actgtgcact
22441 tgaccctctc tcagaaacaa agtgtacgtt gaaatccttc actgtagaaa aaggaatcta
22501 tcaaacttct aactttagag tccaaccaac agaatctatt gttagatttc ctaatattac
22561 aaacttgtgc ccttttggtg aagtttttaa cgccaccaga tttgcatctg tttatgcttg
22621 gaacaggaag agaatcagca actgtgttgc tgattattct gtcctatata attccgcatc
22681 attttccact tttaagtgtt atggagtgtc tcctactaaa ttaaatgatc tctgctttac
22741 taatgtctat gcagattcat ttgtaattag aggtgatgaa gtcagacaaa tcgctccagg
22801 gcaaactgga aagattgctg attataatta taaattacca gatgatttta caggctgcgt
22861 tatagcttgg aattctaaca atcttgattc taaggttggt ggtaattata attacctgta
22921 tagattgttt aggaagtcta atctcaaacc ttttgagaga gatatttcaa ctgaaatcta
22981 tcaggccggt agcacacctt gtaatggtgt tgaaggtttt aattgttact ttcctttaca
23041 atcatatggt ttccaaccca ctaatggtgt tggttaccaa ccatacagag tagtagtact
23101 ttcttttgaa cttctacatg caccagcaac tgtttgtgga cctaaaaagt ctactaattt
23161 ggttaaaaac aaatgtgtca atttcaactt caatggttta acaggcacag gtgttcttac
23221 tgagtctaac aaaaagtttc tgcctttcca acaatttggc agagacattg ctgacactac
23281 tgatgctgtc cgtgatccac agacacttga gattcttgac attacaccat gttcttttgg
23341 tggtgtcagt gttataacac caggaacaaa tacttctaac caggttgctg ttctttatca
23401 ggatgttaac tgcacagaag tccctgttgc tattcatgca gatcaactta ctcctacttg
23461 gcgtgtttat tctacaggtt ctaatgtttt tcaaacacgt gcaggctgtt taataggggc
23521 tgaacatgtc aacaactcat atgagtgtga catacccatt ggtgcaggta tatgcgctag
23581 ttatcagact cagactaatt ctcctcggcg ggcacgtagt gtagctagtc aatccatcat
23641 tgcctacact atgtcacttg gtgcagaaaa ttcagttgct tactctaata actctattgc
23701 catacccaca aattttacta ttagtgttac cacagaaatt ctaccagtgt ctatgaccaa
23761 gacatcagta gattgtacaa tgtacatttg tggtgattca actgaatgca gcaatctttt
23821 gttgcaatat ggcagttttt gtacacaatt aaaccgtgct ttaactggaa tagctgttga
23881 acaagacaaa aacacccaag aagtttttgc acaagtcaaa caaatttaca aaacaccacc
23941 aattaaagat tttggtggtt ttaatttttc acaaatatta ccagatccat caaaaccaag
24001 caagaggtca tttattgaag atctactttt caacaaagtg acacttgcag atgctggctt
24061 catcaaacaa tatggtgatt gccttggtga tattgctgct agagacctca tttgtgcaca
24121 aaagtttaac ggccttactg ttttgccacc tttgctcaca gatgaaatga ttgctcaata
24181 cacttctgca ctgttagcgg gtacaatcac ttctggttgg acctttggtg caggtgctgc
24241 attacaaata ccatttgcta tgcaaatggc ttataggttt aatggtattg gagttacaca
24301 gaatgttctc tatgagaacc aaaaattgat tgccaaccaa tttaatagtg ctattggcaa
24361 aattcaagac tcactttctt ccacagcaag tgcacttgga aaacttcaag atgtggtcaa
24421 ccaaaatgca caagctttaa acacgcttgt taaacaactt agctccaatt ttggtgcaat
24481 ttcaagtgtt ttaaatgata tcctttcacg tcttgacaaa gttgaggctg aagtgcaaat
24541 tgataggttg atcacaggca gacttcaaag tttgcagaca tatgtgactc aacaattaat
24601 tagagctgca gaaatcagag cttctgctaa tcttgctgct actaaaatgt cagagtgtgt
24661 acttggacaa tcaaaaagag ttgatttttg tggaaagggc tatcatctta tgtccttccc
24721 tcagtcagca cctcatggtg tagtcttctt gcatgtgact tatgtccctg cacaagaaaa
24781 gaacttcaca actgctcctg ccatttgtca tgatggaaaa gcacactttc ctcgtgaagg
24841 tgtctttgtt tcaaatggca cacactggtt tgtaacacaa aggaattttt atgaaccaca
24901 aatcattact acagacaaca catttgtgtc tggtaactgt gatgttgtaa taggaattgt
24961 caacaacaca gtttatgatc ctttgcaacc tgaattagac tcattcaagg aggagttaga
25021 taaatatttt aagaatcata catcaccaga tgttgattta ggtgacatct ctggcattaa
25081 tgcttcagtt gtaaacattc aaaaagaaat tgaccgcctc aatgaggttg ccaagaattt
25141 aaatgaatct ctcatcgatc tccaagaact tggaaagtat gagcagtata taaaatggcc
25201 atggtacatt tggctaggtt ttatagctgg cttgattgcc atagtaatgg tgacaattat
25261 gctttgctgt atgaccagtt gctgtagttg tctcaagggc tgttgttctt gtggatcctg
25321 ctgcaaattt gatgaagacg actctgagcc agtgctcaaa ggagtcaaat tacattacac
25381 ataaacgaac ttatggattt gtttatgaga atcttcacaa ttggaactgt aactttgaag
25441 caaggtgaaa tcaaggatgc tactccttca gattttgttc gcgctactgc aacgataccg
25501 atacaagcct cactcccttt cggatggctt attgttggcg ttgcacttct tgctgttttt
25561 cagagcgctt ccaaaatcat aaccctcaaa aagagatggc aactagcact ctccaagggt
25621 gttcactttg tttgcaactt gctgttgttg tttgtaacag tttactcaca ccttttgctc
25681 gttgctgctg gccttgaagc cccttttctc tatctttatg ctttagtcta cttcttgcag
25741 agtataaact ttgtaagaat aataatgagg ctttggcttt gctggaaatg ccgttccaaa
25801 aacccattac tttatgatgc caactatttt ctttgctggc atactaattg ttacgactat
25861 tgtatacctt acaatagtgt aacttcttca attgtcatta cttcaggtga tggcacaaca
25921 agtcctattt ctgaacatga ctaccagatt ggtggttata ctgaaaaatg ggaatctgga
25981 gtaaaagact gtgttgtatt acacagttac ttcacttcag actattacca gctgtactca
26041 actcaattga gtacagacac tggtgttgaa catgttacct tottcatcta caataaaatt
26101 gttgatgagc ctgaagaaca tgtccaaatt cacacaatcg acggttcatc cggagttgtt
26161 aatccagtaa tggaaccaat ttatgatgaa ccgacgacga ctactagcgt gcctttgtaa
26221 gcacaagctg atgagtacga acttatgtac tcattcgttt cggaagagac aggtacgtta
26281 atagttaata gcgtacttct ttttcttgct ttcgtggtat tcttgctagt tacactagcc
26341 atccttactg cgcttcgatt gtgtgcgtac tgctgcaata ttgttaacgt gagtcttgta
26401 aaaccttctt tttacgttta ctctcgtgtt aaaaatctga attcttctag agttcctgat
26461 cttctggtct aaacgaacta aatattatat tagtttttct gtttggaact ttaattttag
26521 ccatggcaga ttccaacggt actattaccg ttgaagagct taaaaagctc cttgaacaat
26581 ggaacctagt aataggtttc ctattcctta catggatttg tcttctacaa tttgcctatg
26641 ccaacaggaa taggtttttg tatataatta agttaatttt cctctggctg ttatggccag
26701 taactttagc ttgttttgtg cttgctgctg tttacagaat aaattggatc accggtggaa
26761 ttgctatcgc aatggcttgt cttgtaggct tgatgtggct cagctacttc attgcttctt
26821 tcagactgtt tgcgcgtacg cgttccatgt ggtcattcaa tccagaaact aacattcttc
26881 tcaacgtgcc actccatggc actattctga ccagaccgct tctagaaagt gaactcgtaa
26941 tcggagctgt gatccttcgt ggacatcttc gtattgctgg acaccatcta ggacgctgtg
27001 acatcaagga cctgcctaaa gaaatcactg ttgctacatc acgaacgctt tcttattaca
27061 aattgggagc ttcgcagcgt gtagcaggtg actcaggttt tgctgcatac agtcgctaca
27121 ggattggcaa ctataaatta aacacagacc attccagtag cagtgacaat attgctttgc
27181 ttgtacagta agtgacaaca gatgtttcat ctcgttgact ttcaggttac tatagcagag
27241 atattactaa ttattatgag gacttttaaa gtttccattt ggaatcttga ttacatcata
27301 aacctcataa ttaaaaattt atctaagtca ctaactgaga ataaatattc tcaattagat
27361 gaagagcaac caatggagat tgattaaacg aacatgaaaa ttattctttt cttggcactg
27421 ataacactcg ctacttgtga gctttatcac taccaagagt gtgttagagg tacaacagta
27481 cttttaaaag aaccttgctc ttctggaaca tacgagggca attcaccatt tcatcctcta
27541 gctgataaca aatttgcact gacttgcttt agcactcaat ttgcttttgc ttgtcctgac
27601 ggcgtaaaac acgtctatca gttacgtgcc agatcagttt cacctaaact gttcatcaga
27661 caagaggaag ttcaagaact ttactctcca atttttctta ttgttgcggc aatagtgttt
27721 ataacacttt gcttcacact caaaagaaag acagaatgat tgaactttca ttaattgact
27781 tctatttgtg ctttttagcc tttctgctat tccttgtttt aattatgctt attatotttt
27841 ggttctcact tgaactgcaa gatcataatg aaacttgtca cgcctaaacg aacatgaaat
27901 ttcttgtttt cttaggaatc atcacaactg tagctgcatt tcaccaagaa tgtagtttac
27961 agtcatgtac tcaacatcaa ccatatgtag ttgatgaccc gtgtcctatt cacttctatt
28021 ctaaatggta tattagagta ggagctagaa aatcagcacc tttaattgaa ttgtgcgtgg
28081 atgaggctgg ttctaaatca cccattcagt acatcgatat cggtaattat acagtttcct
28141 gttcaccttt tacaattaat tgccaggaac ctaaattggg tagtcttgta gtgcgttgtt
28201 cgttctatga agacttttta gagtatcatg acgttcgtgt tgttttagat ttcatctaaa
28261 cgaacaaact aaaatgtctg ataatggacc ccaaaatcag cgaaatgcac cccgcattac
28321 gtttggtgga ccctcagatt caactggcag taaccagaat ggagaacgca gtggggcgcg
28381 atcaaaacaa cgtcggcccc aaggtttacc caataatact gcgtcttggt tcaccgctct
28441 cactcaacat ggcaaggaag accttaaatt ccctogagga caaggogttc caattaacac
28501 caatagcagt ccagatgacc aaattggcta ctaccgaaga gctaccagac gaattcgtgg
28561 tggtgacggt aaaatgaaag atctcagtcc aagatggtat ttctactacc taggaactgg
28621 gccagaagct ggacttccct atggtgctaa caaagacggc atcatatggg ttgcaactga
28681 gggagccttg aatacaccaa aagatcacat tggcacccgc aatcctgcta acaatgctgc
28741 aatcgtgcta caacttcctc aaggaacaac attgccaaaa ggcttctacg cagaagggag
28801 cagaggcggc agtcaagcct cttctcgttc ctcatcacgt agtcgcaaca gttcaagaaa
28861 ttcaactcca ggcagcagta ggggaacttc tcctgctaga atggctggca atggcggtga
28921 tgctgctctt gctttgctgc tgcttgacag attgaaccag cttgagagca aaatgtctgg
28981 taaaggccaa caacaacaag gccaaactgt cactaagaaa tctgctgctg aggcttctaa
29041 gaagcctcgg caaaaacgta ctgccactaa agcatacaat gtaacacaag ctttcggcag
29101 acgtggtcca gaacaaaccc aaggaaattt tggggaccag gaactaatca gacaaggaac
29161 tgattacaaa cattggccgc aaattgcaca atttgccccc agcgcttcag cgttcttcgg
29221 aatgtcgcgc attggcatgg aagtcacacc ttcgggaacg tggttgacct acacaggtgc
29281 catcaaattg gatgacaaag atccaaattt caaagatcaa gtcattttgc tgaataagca
29341 tattgacgca tacaaaacat tcccaccaac agagcctaaa aaggacaaaa agaagaaggc
29401 tgatgaaact caagcottac cocagagaca gaagaaacao caaactgtga ctcttcttcc
29461 tgctgcagat ttggatgatt tctccaaaca attgcaacaa tocatgagca gtgctgactc
29521 aactcaggcc taaactcatg cagaccacac aaggcagatg ggctatataa acgttttcgc
29581 ttttccgttt acgatatata gtctactctt gtgcagaatg aattctcgta actacatagc
29641 acaagtagat gtagttaact ttaatctcac atagcaatct ttaatcagtg tgtaacatta
29701 gggaggactt gaaagagcca ccacattttc accgaggcca cgcggagtac gatcgagtgt
29761 acagtgaaca atgctaggga gagctgccta tatggaagag ccctaatgtg taaaattaat
29821 tttagtagtg ctatccccat gtgattttaa tagcttctta ggagaatgac aaaaaaaaaa
29881 aa
```

SEQ ID NO: 2 is the amino acid sequence of wild-type spike protein from SARS-CoV-2 WA1/2020. The polybasic insert (PRRA) is underlined.

```
      1 MFVFLVLLPL VSSQCVNLIT RTQLPPAYIN SFTRGVYYPD KVFRSSVLHS TQDLFLPFFS
    61 NVTWFHAIHV SGINGTKRED NPVLPENDGV YFASTEKSNI IRGWIFGTTL DSKTQSLLIV
  121 NNATNVVIKV CEFQFCNDPF LGVYYHKNNK SWMESEFRVY SSANNCTFEY VSQPFLMDLE
  181 GKQGNFKNLR EFVFKNIDGY FKIYSKHTPI NLVRDLPQGF SALEPLVDLP IGINITRFQT
  241 LLALHRSYLT PGDSSSGWTA GAAAYYVGYL QPRTFLLKYN ENGTITDAVD CALDPLSETK
  301 CTLKSFTVEK GIYQTSNFRV QPTESIVRFP NITNLCPFGE VFNATRFASV YAWNRKRISN
  361 CVADYSVLYN SASFSTFKCY GVSPTKLNDL CFTNVYADSF VIRGDEVRQI APGQTGKIAD
  421 YNYKLPDDFT GCVIAWNSNN LDSKVGGNYN YLYRLFRKSN LKPFERDIST EIYQAGSTPC
  481 NGVEGENCYF PLQSYGFQPT NGVGYQPYRV VVLSFELLHA PATVCGPKKS TNLVKNKCVN
  541 FNFNGLIGTG VLTESNKKEL PFQQFGRDIA DITDAVRDPQ TLEILDITPC SFGGVSVITP
  601 GTNTSNOVAV LYQDVNCTEV PVAIHADQLT PTWRVYSTGS NVFQTRAGCL IGAEHVNNSY
  661 ECDIPIGAGI CASYQTQTNS PRRARSVASQ SIIAYTMSLG AENSVAYSNN SIAIPTNFTI
  721 SVTTEILPVS MTKTSVDCTM YICGDSTECS NLLLOYGSFC TOLNRALTGI AVEQDKNTQE
  781 VFAQVKQIYK TPPIKDFGGF NFSQILPDPS KPSKRSFIED LLFNKVTLAD AGFIKQYGDC
  841 LGDIAARDLI CAQKENGLTV LPPLLTDEMI AQYTSALLAG TITSGWTFGA GAALQIPFAM
  901 QMAYRENGIG VTONVLYENQ KLIANQFNSA IGKIQDSLSS TASALGKLOD VVNQNAQALN
  961 TLVKQLSSNF GAISSVLNDI LSRLDKVEAE VQIDRLITGR LOSLOTYVTQ QLIRAAEIRA
1021 SANLAATKMS ECVLGQSKRV DFCGKGYHLM SFPQSAPHGV VFLHVTYVPA QEKNFTTAPA
1081 ICHDGKAHFP REGVFVSNGT HWFVTORNFY EPQIITTDNT FVSGNCDVVI GIVNNTVYDP
1141 LQPELDSFKE ELDKYFKNHT SPDVDLGDIS GINASVVNIQ KEIDRLNEVA KNLNESLIDL
1201 QELGKYEQYI KWPWYIWLGF IAGLIAIVMV TIMLCCMTSC CSCLKGCCSC GSCCKFDEDD
1261 SEPVLKGVKL HYT
```

SEQ ID NO: 3 is an exemplary amino acid sequence of a modified SARS-CoV-2 spike protein having a deletion of the polybasic insert (ΔPRRA).

MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSE
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNOVAVLYQDVNCTEVPVAIHADQLTPTWRVYSTGS
NVFQTRAGCLIGAEHVNNSYECDIPIGAGICASYQTQTNSRSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFAQVKQIYKTPPIKDFGGENFSQ
ILPDPSKPSKRSFIEDLLFNKVTLADAGFIKQYGDCLGDIAARDLICAQKENGLTVLPPLLTDEMIAQYTSALLAGTITS
GWTFGAGAALQIPFAMQMAYRFNGIGVTQNVLYENQKLIANQFNSAIGKIQDSLSSTASALGKLQDVVNQNAQALNTLVK
QLSSNFGAISSVLNDILSRLDKVEAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGTHWFVTQRNFYEPQIITTDNTFVSG
NCDVVIGIVNNTVYDPLQPELDSFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCCKFDEDDSEPVLKGVKLHYT

SEQ ID NO: 4 is the amino acid sequence of wild-type Nsp1 from SARS-CoV-2 WA1/2020.

MESLVPGFNEKTHVQLSLPVLQVRDVLVRGFGDSVEEVLSEARQHLKDGT
CGLVEVEKGVLPQLEQPYVFIKRSDARTAPHGHVMVELVAELEGIQYGRS
GETLGVLVPHVGEIPVAYRKVLLRKNGNKGAGGHSYGADLKSFDLGDELG
TDPYEDFQENWNTKHSSGVTRELMRELNGG

SEQ ID NO: 5 is an exemplary amino acid sequence of a modified SARS-CoV-2 Nsp1 having the K164A and H165A substitutions (underlined).

MESLVPGFNEKTHVQLSLPVLQVRDVLVRGFGDSVEEVLSEARQHLKDGT
CGLVEVEKGVLPQLEQPYVFIKRSDARTAPHGHVMVELVAELEGIQYGRS
GETLGVLVPHVGEIPVAYRKVLLRKNGNKGAGGHSYGADLKSFDLGDELG
TDPYEDFQENWNTAASSGVTRELMRELNGG

SEQ ID NOs: 6-32 are primer sequences (see Example 1).

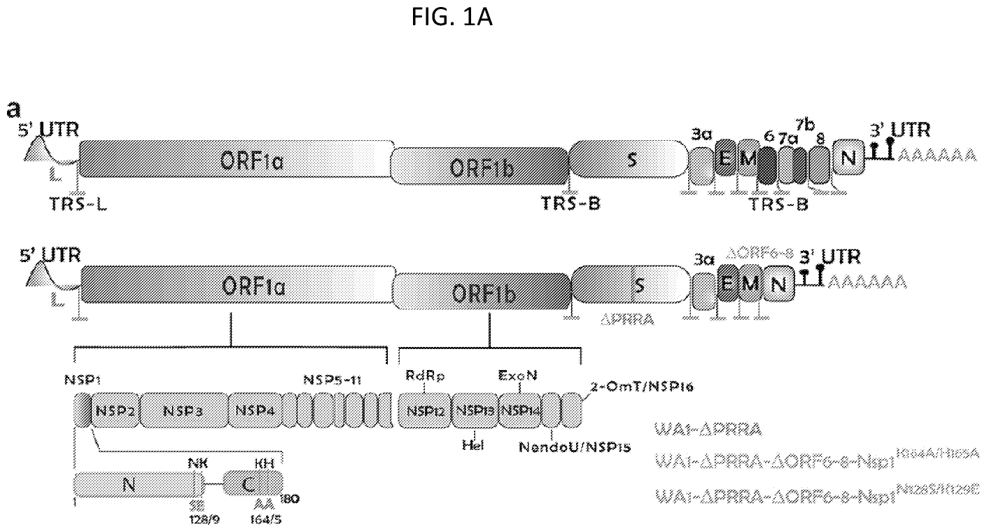

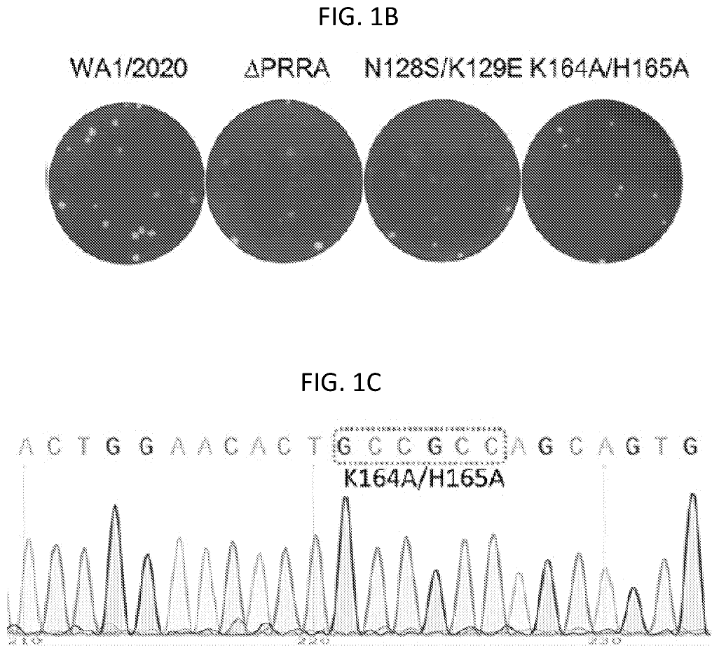

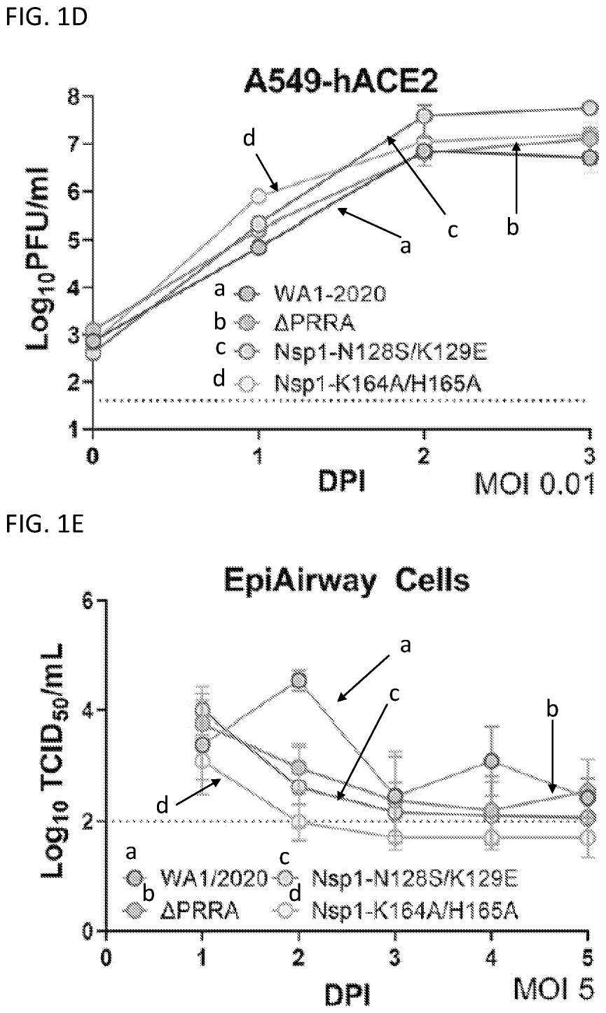

SEQ ID NO: 33 is a nucleic acid fragment of a SARS-CoV-2 Nsp1 coding sequence (see FIG. 1C).

### DETAILED DESCRIPTION

### I. Abbreviations

BALF broncho-alveolar lavage fluid
COVID-19 coronavirus disease 2019
DPC days post-challenge
DPE days post-exposure
DPI days post-infection
ELISA enzyme-linked immunosorbent assay
FFU focus-forming assay
FFPE formalin-fixed paraffin-embedded
FFU focus-forming units
FRNT₅₀ 50% focus reduction neutralization titer
hACE2 human angiotensin converting enzyme 2
HE haematoxylin and eosin
IFN interferon
ISH in situ hybridization
LAV live attenuated virus
MOI multiplicity of infection
MPE months post-exposure
nAb neutralizing antibody
NP nucleocapsid protein
NW nasal wash
ORF open reading frame
PFA paraformaldehyde
PFU plaque forming units
ProSPC prosurfactant protein C
RBD receptor binding domain
RT room temperature
SARS-CoV-2 severe acute respiratory syndrome coronavirus 2
sgRNA subgenomic RNA
TCIDso tissue culture infectious dose 50
TLR toll-like receptor
VOC variant of concern

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Adjuvant: A component of an immunogenic composition used to enhance antigenicity. In some aspects, an adjuvant can include a suspension of minerals (alum, aluminum hydroxide, or phosphate) on which antigen is adsorbed; or water-in-oil emulsion, for example, in which antigen solution is emulsified in mineral oil (Freund's incomplete adjuvant), sometimes with the inclusion of killed mycobacteria (Freund's complete adjuvant) to further enhance antigenicity (inhibits degradation of antigen and/or causes influx of macrophages). In some aspects, the adjuvant used in a disclosed immunogenic composition is a combination of lecithin and carbomer homopolymer (such as the ADJUPLEX™ adjuvant available from Advanced BioAdjuvants, LLC; see also Wegmann, Clin Vaccine Immunol 22(9): 1004-1012, 2015). Additional adjuvants for use in the disclosed immunogenic compositions include the QS21 purified plant extract, Matrix M, ASO1, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary biological adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill is familiar with adjuvants (see, e.g., Singh (ed.) Vaccine Adjuvants and Delivery Systems. Wiley-Interscience, 2007).

Administration: To provide or give a subject an agent, such as an immunogenic composition provided herein, by any effective route. Exemplary routes of administration include, but are not limited to, intranasal, oral, injection (such as subcutaneous, intramuscular, intradermal, intraperitoneal, intravenous, and intratumoral), sublingual, rectal, transdermal, vaginal and inhalation routes.

Attenuated virus: A virus that has decreased virulence compared to a reference virus under similar conditions of infection. Attenuation usually is associated with decreased virus replication as compared to replication of a reference wild-type virus under similar conditions of infection. In some hosts (typically non-natural hosts, including experimental animals), disease is not evident during infection with a reference virus in question, and restriction of virus replication can be used as a surrogate marker for attenuation. In some aspects, a disclosed attenuated SARS-CoV-2 exhibits at least about 10-fold or greater decrease, such as at least about 20-fold, 40-fold, 60-fold, 80-fold or 100-fold or greater decrease in virus titer, such as in the upper or lower respiratory tract of a mammal, compared to non-attenuated, wild type virus titer in the upper or lower respiratory tract of a mammal of the same species under the same conditions of infection.

Codon-optimized: A nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Conservative variant: A protein containing conservative amino acid substitutions that do not substantially affect or decrease the function of a protein, such as a coronavirus spike protein. “Conservative” amino acid substitutions are those substitutions that do not substantially affect or decrease a function of a protein, such as the ability of the protein to elicit an immune response when administered to a subject. The term conservative variant also includes the use of a substituted amino acid in place of an unsubstituted parent amino acid. Furthermore, individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of a protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2 (including variants thereof, such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2) and omicron (BA.1.1.529, BA.1, BA.1.1, BA.2, BA.3, BA.4 and BA.5)), and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), and human coronavirus NL63 (NL63-CoV).

COVID-19: The disease caused by the coronavirus SARS-CoV-2.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Effective amount: An amount of agent (such as a live-attenuated SARS-CoV-2) that is sufficient to elicit a desired response, such as an immune response in a subject. A “therapeutically effective amount” can be the amount necessary to inhibit SARS-CoV-2 replication or to treat COVID-19 in a subject with an existing SARS-CoV-2 infection. A “prophylactically effective amount” refers to an amount of an agent or composition necessary to inhibit or prevent establishment of an infection, such infection by SARS-CoV-2. It is understood that obtaining a protective immune response against SARS-CoV-2 can require multiple administrations of a disclosed immunogen (e.g., live-attenuated SARS-CoV-2), and/or administration of a disclosed immunogen as the “prime” in a prime boost protocol wherein the boost immunogen can be different from the prime immunogen (such as a second SARS-CoV-2 vaccine). Alternatively, a disclosed immunogen be administered as a boost dose following a prime dose of a different SARS-CoV-2 vaccine. Accordingly, an effective amount of a disclosed immunogen can be the amount of the immunogen sufficient to elicit a priming immune response in a subject that can be subsequently boosted with the same or a different immunogen to elicit a protective immune response. Similarly, an effective amount of a disclosed immunogen can be the amount of immunogen sufficient to elicit a protective immune response when administered following a prime immunization.

In one example, a desired response is to elicit an immune response that inhibits or prevents SARS-CoV-2 infection. The SARS-CoV-2 infected cells do not need to be completely eliminated or prevented for the composition to be effective. For example, administration of an effective amount of an immunogen or immunogenic composition can elicit an immune response that decreases the number of SARS-CoV-2 infected cells (or prevents the infection of cells) by a desired amount, for example, by at least 50%, at least 60%, at least 70%, at least 80%, at least 90%, at least 95%, at least 98%, or even at least 100% (elimination or prevention of detectable SARS-CoV-2 infected cells), as compared to the number of SARS-CoV-2 infected cells in the absence of the immunization or other suitable control.

Heterologous: Originating from a different genetic source. A heterologous gene included in a recombinant genome is a gene that does not originate from that genome.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny is included when the term “host cell” is used.

Immune response: A response of a cell of the immune system, such as a B cell, T cell, or monocyte, to a stimulus. In some aspects, the response is specific for a particular antigen (an “antigen-specific response”), such as SARS-CoV-2 or a SARS-CoV-2 protein. In some aspects, the immune response is a T cell response, such as a CD4+ response or a CD8+ response. In other aspects, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition. Together, the prime and boost immunizations produce the desired immune response in the subject.

Immunize: To render a subject protected from infection by a particular infectious agent, such as SARS-CoV-2. Immunization does not require 100% protection. In some examples, immunization provides at least 50%, at least 60%, at least 70%, at least 80%, at least 90% or at least 95% protection against infection compared to infection in the absence of immunization.

Immunogenic composition: A composition that includes an immunogen (such as a live-attenuated SARS-CoV-2) or a nucleic acid molecule or vector encoding an immunogen, that elicits a measurable CTL response against the immunogen, and/or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition can include the immunogen in a pharmaceutically acceptable carrier and may also include other agents, such as an adjuvant.

Isolated: An “isolated” biological component has been substantially separated or purified away from other biological components, such as other biological components in which the component occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Neutralizing antibody (nAb): An antibody that reduces the infectious titer of an infectious agent by binding to a specific antigen on the infectious agent, such as a virus (e.g., a coronavirus). In some aspects, an antibody that is specific for SARS-CoV-2 or a protein thereof (such as a spike protein) neutralizes the infectious titer of SARS-CoV-2. For example, an antibody that neutralizes SARS-CoV-2 may interfere with the virus by binding it directly and limiting entry into cells. Alternately, a neutralizing antibody may interfere with one or more post-attachment interactions of the pathogen with a receptor, for example, by interfering with viral entry using the receptor. In some aspects, a SARS-CoV-2 neutralizing antibody inhibits SARS-CoV-2 infection of cells, for example, by at least 50%, by at least 60%, by at least 70%, by at least 80%, by at least 90%, or by at least 95% compared to a control antibody.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “polynucleotide.” A nucleic acid molecule is usually at least 10 bases in length, unless otherwise specified. The term includes single- and double-stranded forms of DNA. A nucleic acid molecule may include either or both naturally occurring and modified nucleotides linked together by naturally occurring and/or non-naturally occurring nucleotide linkages.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Permissive cell: A cell in which a virus (such as SARS-CoV-2) can have a productive infection, which includes being able to infect the cell and replicate in the cell. Non-limiting examples of cells permissive for SARS-CoV-2 include Vero cells, BGMK cells. CV-1 cells, LLC-MK2 cells, A549 cells, RhMK cells and HeLa cells (see, e.g., Wang et al., Emerg Infect Dis 27(5):1380-1392, 2021).

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens (such as live-attenuated SARS-CoV-2) and immunogenic compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular aspects, suitable for administration to a subject the carrier may be sterile, and/or suspended or otherwise contained in a unit dosage form containing one or more measured doses of the composition suitable to elicit the desired anti-SARS-CoV-2 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Prime-boost immunization: An immunization protocol including administration of a first immunogenic composition (the prime immunization) followed by administration of a second immunogenic composition (the boost immunization) to a subject to induce a desired immune response. A suitable time interval between administration of the prime and the boost, and examples of such timeframes are disclosed herein. In some aspects, the prime, the boost, or both the prime and the boost additionally include an adjuvant. In some examples, the immunogenic composition used for the prime and the boost is the same. In other examples, different immunogenic compositions are used for the prime and boost doses.

Recombinant: A recombinant nucleic acid molecule, protein or virus is one that has been produced by recombinant DNA methods, typically from cloned cDNA(s). The cDNA sequence(s) may be identical to that of a biologically-derived molecule(s), or may contain a sequence(s) that is not naturally-occurring: for example, includes one or more nucleic acid substitutions, deletions or insertions, and/or has a sequence that is made by an artificial combination of two otherwise separated segments of sequence. This artificial combination can be accomplished, for example, by chemical synthesis, targeted mutation of a naturally occurring nucleic acid molecule or protein, or, artificial manipulation of isolated segments of nucleic acids, for example, by genetic engineering techniques.

Reverse genetics plasmids: Plasmids containing cDNA corresponding to an RNA virus genome or a fragment of the genome. As described in Example 1, reverse genetics plasmids that each contain a fragment of the SARS-CoV-2 genome can be used to introduce specific mutations in one or more virus genes and generate a modified and infectious SARS-CoV-2 (see, e.g., Xie et al., Cell Host Microbe 27:841-848 e843, 2020; Xie et al., Nat Protoc 16:1761-1784, 2021, which describe the 7 plasmid system used herein). Other SARS-CoV-2 reverse genetics plasmids have been previously described and can be used to generate the attenuated SARS-CoV-2 described herein (see, e.g., Mdlade et al., EMBO Rep 23:e53820, 2022; Rihn et al., PLoS Biol 19(2):e3001091, 2021; Torii et al., Cell Rep 35:109014, 2021).

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

SARS-CoV-2: A coronavirus of the genus betacoronavirus that first emerged in humans in 2019. This virus is also known as Wuhan coronavirus, 2019-nCoV, or 2019 novel coronavirus. The term “SARS-CoV-2” includes variants thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2) and omicron (B.1.1.529 and BA lineages). Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′. A SARS-CoV-2 variant of concern (VOC) refers to a SARS-CoV-2 variant associated with increased transmissibility, more severe disease (such as increased hospitalizations or deaths), significant reduction in neutralization by antibodies generated during previous infection or vaccination, reduced effectiveness of treatments or vaccines and/or diagnostic detection failures (see cdc.gov/coronavirus/2019-ncov/variants/variant-classifications).

SARS Spike (S) protein: A class I fusion glycoprotein initially synthesized as a precursor protein of approximately 1256 amino acids for SARS-CoV, and 1273 amino acids for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer, thereby forming a trimer of heterodimers. The S1 subunit is distal to the virus membrane and contains the receptor-binding domain (RBD) that is believed to mediate virus attachment to its host receptor. The S2 subunit is believed to contain the fusion protein machinery, such as the fusion peptide. S2 also includes two heptad-repeat sequences (HR1 and HR2) and a central helix typical of fusion glycoproteins, a transmembrane domain, and a cytosolic tail domain.

Subject: Living multi-cellular vertebrate organisms, a category that includes both human and veterinary subjects, including human and non-human mammals, such as birds, pigs, mice, rats, rabbits, sheep, horses, cows, dogs, cats and non-human primates. In some aspects, the subject is a human. In some examples, a subject who is in need of inhibiting or preventing a SARS-CoV-2 infection is selected. For example, the subject can be uninfected and at risk of SARS-CoV-2 infection.

Under conditions sufficient for: A phrase that is used to describe any environment that permits a desired activity.

Unit dosage form: A physically discrete unit, such as a capsule, tablet, or solution, that is suitable as a unitary dosage for a human patient, each unit containing a predetermined quantity of one or more active ingredient(s) (such as a live-attenuated SARS-CoV-2) calculated to produce a therapeutic effect, in association with at least one pharmaceutically acceptable diluent or carrier, or combination thereof.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a virus (such as an attenuated and/or recombinant virus), a polynucleotide (such as a nucleic acid encoding a disclosed antigen), a peptide or polypeptide (such as a disclosed antigen), a cell or one or more cellular constituents. In one specific, non-limiting example, a vaccine reduces the severity of the symptoms associated with SARS-CoV-2 infection and/or decreases the viral load compared to a control. In another non-limiting example, a vaccine reduces SARS-CoV-2 infection and/or transmission compared to a control. A live-attenuated vaccine (LAV) refers to a vaccine that includes a recombinant virus containing one or more modifications that weaken the virus (e.g., mutations that reduce replication of the virus and/or render the virus less capable of causing disease). In the context of the present disclosure, a live-attenuated SARS-CoV-2 is a modified SARS-CoV-2 that remains capable of infecting and replicating in mammalian cells, but includes a combination of modifications that attenuate the virus.

Vector: An entity containing a DNA or RNA molecule bearing a promoter(s) that is operationally linked to the coding sequence of a protein (such as an immunogenic protein) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. Non-limiting examples of viral vectors include adenovirus vectors, adeno-associated virus (AAV) vectors, and poxvirus vectors (e.g., vaccinia, fowlpox).

### III. Live-Attenuated SARS-CoV-2

Engineered SARS-CoV-2 variants having a combination of attenuating modifications, and their use as live-attenuated SARS-CoV-2 vaccines, are described herein. The recombinant genome of the live-attenuated SARS-CoV-2 encodes a modified spike (S) protein with a deletion of the polybasic site (ΔPRRA) and encodes a modified non-structural protein 1 (Nsp1) with K164A and H165A substitutions. The recombinant genome also includes a mutation(s) (such as a deletion) that prevents expression of open reading frames (ORFs) 6, 7a, 7b and 8. The disclosed live-attenuated SARS-CoV-2 retain the capacity to infect and replicate in mammalian cells.

Removal of the polybasic site (PRRA) impairs the ability of SARS-CoV-2 to infect the lung. Non-structural protein 1 (Nsp1) and accessory proteins encoded by ORFs 6, 7a, 7b and 8 are potent interferon (IFN) antagonists that subvert the host innate immune response to promote virus infection. In addition, clinical isolates containing a 382-nucleotide deletion in ORF8 are associated with a milder infection. Thus, mutation of Nsp1 and deletion or modification of ORFs 6, 7a, 7b and 8 as disclosed herein impair the ability of SARS-CoV-2 to antagonize IFN and thereby inhibit SARS-CoV-2 replication.

Unlike SARS-CoV-2 vaccines that express only the spike protein as immunogen, LAVs confer broader and/or more durable protection because the whole organism is recognized by the host immune system. As disclosed herein, live-attenuated SARS-CoV-2 having the above-listed combination of attenuating modifications exhibited 100- to 1000-fold lower titers than the corresponding wild-type virus, while also eliciting a significantly subdued proinflammatory response. In particular, inoculation with only 100 PFU of the live-attenuated SARS-CoV-2 resulted in a potent humoral immune response, prevented lung pathology, and provided complete protection against weight loss and pneumonia following SARS-CoV-2 challenge in an animal model. The observed protection was accompanied by more than 5-log₁₀ reductions in viral loads in the lung and trachea following challenge. Immunized animals also displayed over 4-log₁₀ reductions in nasal viral load, indicating possible reduction of virus shedding and transmission. This feature is especially desirable for a SARS-CoV-2 vaccine to quell the pandemic. It is known that natural infection by SARS-CoV-2 induces both mucosal antibody responses and systemic antibody responses (Smith et al., Nat Immunol 22:1428-1439, 2021). Secretory immunoglobulin A (IgA) is thought to play a major role in protecting the upper and lower respiratory tract from acute infection. However, vaccines that are administered intramuscularly or intradermally potently induce IgG, but very little secretory IgA (Krammer et al., Nature 586:516-527, 2020; Azzi et al., EBioMedicine 75:103788, 2022). By contrast, intranasal administration of a vaccine, such as the live-attenuated SARS-CoV-2 disclosed herein, is expected to induce more potent mucosal immunity (King et al., Vaccines 9(8):881, 2021; Alu et al., EBioMedicine 76:103841, 2022). Furthermore, it is believed that compared to spike-based vaccines, the disclosed live-attenuated SARS-CoV-2 will activate broader cellular immunity that is cross-protective against variants of concern because replication of the attenuated virus offers many more targets to derive T cell epitopes. Thus, the disclosed live-attenuated SARS-CoV-2 represents a safe and effective vaccine against SARS-CoV-2 infection and COVID-19 disease.

Provided herein is a live-attenuated SARS-CoV-2 having a recombinant genome encoding a modified spike (S) protein with a deletion of the polybasic site (ΔPRRA) corresponding to residues 681-684 of the reference sequence set forth as SEQ ID NO: 2, and encoding a modified non-structural protein 1 (Nsp1) with K164A and H165A substitutions corresponding to the reference sequence set forth as SEQ ID NO: 4. The recombinant genome further includes a mutation(s) that prevents expression of open reading frames (ORFs) 6, 7a, 7b and 8. The disclosed live-attenuated SARS-CoV-2 are capable of infecting and replicating in mammalian cells. Although the above-listed modifications are described with reference to the Wuhan strain, the same modifications can be made in any SARS-CoV-2 variant as the polybasic site, the Nsp1 K164/H165 residues and ORFs 6-8 are conserved among all major SARS-CoV-2 variants of concern (VOC).

In some aspects, the live-attenuated SARS-CoV-2 is a Wuhan strain SARS-CoV-2. In other aspects the live-attenuated SARS-CoV-2 is a variant of the Wuhan strain, such as a variant from the Alpha, Beta, Delta, Gamma, Epsilon, Eta, Iota, Kappa, Mu, Zeta or Omicron lineage. In some examples, the live-attenuated SARS-CoV-2 is a SARS-CoV-2 VOC, such as a VOC from the Delta lineage (e.g., B.1.617.2, or AY lineages) or the Omicron lineage (e.g., B.1.1.529, BA.1, BA.1.1, BA.2, BA.3, BA.4 or BA.5).

In some aspects, the amino acid sequence of the modified S protein of the live-attenuated SARS-CoV-2 is at least 80%, at least 85%, at least 90%, at least 95%, at least 96%, at least 97%, at least 98% or at least 99% identical to SEQ ID NO: 2 and has the deletion of the polybasic insert. In some examples, the amino acid sequence of the modified S protein comprises or consists of SEQ ID NO: 3.

In some aspects, the amino acid sequence of the modified Nsp1 of the live-attenuated SARS-CoV-2 is at least 80%, at least 85%, at least 90%, at least 95%, at least 96%, at least 97%, at least 98% or at least 99% identical to SEQ ID NO: 4 and includes the K164A and H165A substitutions. In some examples, the amino acid sequence of the modified Nsp1 comprises or consists of SEQ ID NO: 5.

In some aspects, the mutation that prevents expression of ORFs 6, 7a, 7b and 8 is a complete deletion or a partial deletion of ORFs 6, 7a, 7b and 8 that prevents expression of ORFs 6, 7a, 7b and 8.

The live-attenuated SARS-CoV-2 may include one or more additional modifications, such as additional attenuating modifications. In some aspects, the live-attenuated SARS-CoV-2 includes one or modifications (such as one or more deletions or substitutions) in the Nsp14, Nsp16, Nsp5 and/or E gene of SARS-CoV-2. In some examples, the modification of Nsp14 is a modification that inhibits N7-methyltransferase activity of the protein, such as a Y420A substitution (Pan et al., mBio 13(1):e0366221, 2022). In some examples, the modification of Nsp16 inhibits 2′O-methyltransferase activity of the protein, such as a D130A substitution or a deletion of the Nsp16 gene (Ye et al., Cell Mol Immunol 19(5):588-601, 2022).

### IV. Immunogenic Compositions

Immunogenic compositions that include a disclosed immunogen (e.g., a live-attenuated SARS-CoV-2), and a pharmaceutically acceptable carrier are also provided. Such compositions can be administered to subjects by a variety of administration modes, for example, intranasal, onto the tonsils, inhalation, oral, intramuscular, subcutaneous, intravenous, intra-arterial, intra-articular, intraperitoneal, or parenteral routes. Methods for preparing administrable compositions are described in more detail in such publications as Remingtons Pharmaceutical Sciences, 19^{th} Ed., Mack Publishing Company, Easton, Pennsylvania, 1995.

An immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffered saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The pharmaceutical composition may optionally include an adjuvant to enhance an immune response of the host. Suitable adjuvants are, for example, toll-like receptor agonists, alum, AlPO₄, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product. In some aspects, an adjuvant is not required and is thus not administered with the live-attenuated SARS-CoV-2.

In some aspects, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which elicits an immune response without significant, adverse side effects. In some examples, the dose is about 1×10², 1×10³, 1×10⁴, 1×10⁵ or 1×10⁶ viral particles, such as about 1×10⁴ to about 10⁶ viral particles, such as about 5×10⁴ to about 5×10⁵ viral particles or about 1×10⁵ viral particles.

In some aspects, the composition can be provided in unit dosage form for use to elicit an immune response in a subject, for example, to prevent SARS-CoV-2 infection in the subject. A unit dosage form contains a suitable single preselected dosage for administration to a subject, or suitable marked or measured multiples of two or more preselected unit dosages, and/or a metering mechanism for administering the unit dose or multiples thereof. In some examples, the unit dosage is about 1×10⁴ to about 10⁶ viral particles, such as about 5×10⁴ to about 5×10⁵ viral particles. In specific examples, the unit dosage is about 1×10⁵ viral particles.

In some aspects, the immunogenic composition is formulated for intranasal administration.

### V. Methods of Eliciting an Immune Response

The disclosed live-attenuated SARS-CoV-2 and compositions including same, can be used in methods of inducing an immune response to SARS-CoV-2 to prevent, inhibit (including inhibiting transmission), and/or treat a SARS-CoV-2 infection.

Provided herein are methods of eliciting an immune response against SARS-CoV-2 in a subject. In some aspects, the method includes administering to the subject an effective amount of a live-attenuated SARS-CoV-2 or immunogenic composition disclosed herein. In some examples, the live-attenuated SARS-CoV-2 or immunogenic composition is administered intranasally (such as in a spray) or orally (such as by using enteric-coated tablets).

When inhibiting, treating, or preventing SARS-CoV-2 infection, the methods can be used either to avoid infection in a SARS-CoV-2 seronegative subject (e.g., by inducing an immune response that protects against SARS-CoV-2 infection), or to treat existing infection in a SARS-CoV-2 seropositive subject.

To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize SARS-CoV-2 infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

In some aspects, the effective amount of the live-attenuated SARS-CoV-2 or the immunogenic composition is administered in a single dose. In other aspects, the live-attenuated SARS-CoV-2 or the immunogenic composition is administered as part of a prime-boost immunization protocol. In some examples, the live-attenuated SARS-CoV-2 or the immunogenic composition is administered as both the prime dose and the boost dose. In other examples, the live-attenuated SARS-CoV-2 or the immunogenic composition is administered as the prime dose and a second SARS-CoV-2 vaccine is administered as the boost dose. In yet other examples, the live-attenuated SARS-CoV-2 or the immunogenic composition is administered as the boost dose and a second SARS-CoV-2 vaccine is administered as the prime dose. In some examples, the second SARS-CoV-2 vaccine is administered intramuscularly.

In certain aspects, combinatorial immunogenic compositions and coordinate immunization protocols employ separate immunogens or formulations, each directed toward eliciting an anti-SARS-CoV-2 immune response, such as an immune response to SARS-CoV-2 spike protein. Separate immunogenic compositions that elicit the anti-SARS-CoV-2 immune response can be combined in a polyvalent immunogenic composition administered to a subject in a single immunization step, or they can be administered separately (in monovalent immunogenic compositions) in a coordinate immunization protocol.

In one aspect, a suitable immunization regimen includes at least two separate inoculations with one or more immunogenic compositions including a disclosed live-attenuated SARS-CoV-2 with a second inoculation being administered more than about two weeks, about three weeks, or about four weeks, such about three to eight weeks, following the first inoculation. A third inoculation can be administered several months after the second inoculation, and in specific aspects, more than about four months, five months, or six months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of SARS-CoV-2 infection, improvement in disease state (e.g., reduction in viral load), or reduction in transmission frequency. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such as one to five, or more. Different dosages can be used in a series of sequential inoculations. For example, a relatively large dose in a primary inoculation and then a boost with relatively smaller doses. The immune response against the selected antigenic surface can be elicited by one or more inoculations of a subject.

In several aspects, a disclosed immunogen can be administered to the subject simultaneously with the administration of an adjuvant. In other aspects, the immunogen can be administered to the subject after the administration of an adjuvant and within a sufficient amount of time to elicit the immune response. In other aspects, no adjuvant is administered.

SARS-CoV-2 infection does not need to be completely inhibited for the methods to be effective. For example, elicitation of an immune response to SARS-CoV-2 can reduce or inhibit SARS-CoV-2 infection by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-CoV-2 infected cells), as compared to SARS-CoV-2 infection in the absence of immunization. In additional examples, SARS-CoV-2 replication can be reduced or inhibited by the disclosed methods. SARS-CoV-2 replication does not need to be completely eliminated for the method to be effective. For example, the immune response elicited using one or more of the disclosed immunogens can reduce SARS-CoV-2 replication by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-CoV-2 replication), as compared to SARS-CoV-2 replication in the absence of the immune response.

Following immunization of a subject, serum can be collected from the subject at appropriate time points, frozen, and stored for neutralization testing. Methods to assay for neutralization activity, include, but are not limited to, plaque reduction neutralization (PRNT) assays, microneutralization assays, flow cytometry-based assays, single-cycle infection assays, and pseudovirus neutralization assays.

### VI. Nucleic Acid Molecules, Reverse Genetics Plasmids and Kits

Also provided are nucleic acid molecules that include the complement of the recombinant genome of a live-attenuated SARS-CoV-2 disclosed herein. In some aspects, a single nucleic acid molecule includes the complement of the SARS-CoV-2 recombinant genome. For example, the single nucleic acid molecule can include a bacterial artificial chromosome (BAC), a yeast artificial chromosome (YAC), a human artificial chromosome (HAC), a P1-derived artificial chromosome (PAC), a cosmid, or a plasmid. In other aspects, a plurality of nucleic acid molecules, such as at least 2, at least 3, at least 4, at least 5, at least 6 or at least 7 nucleic acid molecules collectively include the complete recombinant SARS-CoV-2 genome. In some examples, the plurality of nucleic acid molecules are plasmids.

Further provided are a collection of reverse genetics plasmids that include the complement of the recombinant genome of a live-attenuated SARS-CoV-2 disclosed herein. As described in Example 1, reverse genetics plasmids that each contain a fragment of the SARS-CoV-2 genome can be used to introduce specific mutations in one or more virus genes and generate a modified and infectious SARS-CoV-2 (see, e.g., Xie et al., Cell Host Microbe 27:841-848 e843, 2020; Xie et al., Nat Protoc 16:1761-1784, 2021, which describe the 7 plasmid system used herein). Other SARS-CoV-2 reverse genetics plasmids have been previously described and can be used to generate the attenuated SARS-CoV-2 described herein (see, e.g., Mélade et al., EMBO Rep 23:e53820, 2022; Rihn et al., PLoS Biol 19(2):e3001091, 2021; Torii et al., Cell Rep 35:109014, 2021).

A method of producing a live-attenuated SARS-CoV-2 is also provided herein. In some aspects, the method includes transfecting permissive cells with the reverse genetics plasmids described herein; culturing the transfected cells under conditions sufficient to allow for replication of the attenuated SARS-CoV-2; and isolating the attenuated SARS-CoV-2 from the cell culture. The permissive cells are any cells susceptible to infection by SARS-CoV-2 and that support SARS-CoV-2 replication. In some examples, the permissive cells are mammalian cells, such as, but not limited to, Vero cells, BGMK cells, CV-1 cells, LLC-MK2 cells, A549 cells, RhMK cells and HeLa cells (see, e.g., Wang et al., Emerg Infect Dis 27(5):1380-1392, 2021, for a list of SARS-CoV-2 permissive cells). Attenuated SARS-CoV-2 produced by the disclosed method are further provided.

Also provided are kits, such as kits for production of an attenuated SARS-CoV-2 disclosed herein. In some aspects, the kit includes a collection of reverse genetics plasmids disclosed herein. In some examples, the kit further includes transfection reagent(s), cultured cells (such as cells permissive for infection by SARS-CoV-2), cell culture media and/or cell culture flasks. In some examples, components of a kit are present in separate vials or containers, which in some examples are composed of glass, metal, or plastic.

### EXAMPLES

The following examples are provided to illustrate particular features of certain aspects of the disclosure, but the scope of the claims should not be limited to those features exemplified.

### Example 1: Materials and Methods (Part 1)

This example describes the materials and methods used for the studies described in Examples 2-5.

Reagent List

| REAGENT or RESOURCE | SOURCE | IDENTIFIER |
|---|---|---|
| Bacterial and Virus Strains | | |
| SARS-Coronavirus 2, Isolate USA-WA1/2020 (SEQ ID NO: 1) | BEI | Cat # NR-52281 |
| WA1-ΔPRRA | Described herein | Cat # NR-56461 |
| WA1-ΔPRRA-ORF6-8-Nsp1^{N128S/K129E} | Described herein | |
| WA1-ΔPRRA-ORF6-8-Nsp1^{K164A/H165A} | Described herein | |
| Endura ™ Chemically Competent Cells | Lucigen | Cat # 60240-2 |
| TransforMax ™ EPI300 ™ Competent Cells | Lucigen | Cat # C300C105 |
| Chemicals, Peptides, and Recombinant Proteins | | |
| Lipofectamine-3000 Transfection Reagent | ThermoFisher | Cat # L3000015 |
| mMESSAGE mMACHINE ™ T7 Transcription Kit | ThermoFisher | Cat # |
| | | AM1344 |
| Luciferase Assay System | Promega | Cat # E1501 |
| Experimental Models: Cell Lines | | |
| Lenti-X ™ 293T Cell Line | Takara | Cat # 632180 |
| EpiAirway cells | MatTek | AIR-100-HCF |
| A549-hACE2 | BEI | Cat # |
| | | NR-53821 |
| Vero (C1008) E6 | ATCC | Cat # CRL-1586 |
| Oligonucleotides | | |
| Primer M13 F | Facility for | |
| GTAAAACGACGGCCAGT (SEQ ID NO: 6) | Biotechnology | |
| | Resources, FDA | |
| Primer N128S/K129Ef | Facility for | |
| TAAGAACGGTAGTGAGGGAGCTGGTGGCCATAGTTA | Biotechnology | |
| (SEQ ID NO: 7) | Resources, FDA | |
| Primer N128S/K129E r | Facility for | |
| CACCAGCTCCCTCACTACCGTTCTTACGAAGAAGAA | Biotechnology | |
| (SEQ ID NO: 8) | Resources, FDA | |
| Primer K164A/H165Af | Facility for | |
| AAAACTGGAACACTGCCGCCAGCAGTGGTGTTACCCG | Biotechnology | |
| TGA (SEQ ID NO: 9) | Resources, FDA | |
| Primer K164A/H165Ar | Facility for | |
| GGGTAACACCACTGCTGGCGGCAGTGTTCCAGTTTTC | Biotechnology | |
| TTGAA (SEQ ID NO: 10) | Resources, FDA | |
| Primer NheIr | Facility for | |
| CACGAGCAGCCTCTGATGCA (SEQ ID NO: 11) | Biotechnology | |
| | Resources, FDA | |
| Primer ΔPRRA-f | Facility for | |
| ACTCAGACTAATTCTCGTAGTGTAGCTAGTCAATC | Biotechnology | |
| (SEQ ID NO: 12) | Resources, FDA | |
| Primer ΔPRRA-r | Facility for | |
| ACTAGCTACACTACGAGAATTAGTCTGAGTCTGAT | Biotechnology | |
| (SEQ ID NO: 13) | Resources, FDA | |
| Primer Bgl II r | Facility for | |
| CAGCATCTGCAAGTGTCACT (SEQ ID NO: 14) | Biotechnology | |
| | Resources, FDA | |
| Primer Mf | Facility for | |
| TTAATTTTAGCCATGGCAGA (SEQ ID NO: 15) | Biotechnology | |
| | Resources, FDA | |
| Primer ORF68f | Facility for | |
| TTTGCTTGTACAGTAAACGAACAAACTAAAATGTC | Biotechnology | |
| (SEQ ID NO: 16) | Resources, FDA | |
| Primer ORF68r | Facility for | |
| TTTTAGTTTGTTCGTTTACTGTACAAGCAAAGCAA | Biotechnology | |
| (SEQ ID NO: 17) | Resources, FDA | |
| Primer AvrIIr | Facility for | |
| GAAGTCCAGCTTCTGGCCCA (SEQ ID NO: 18) | Biotechnology | |
| | Resources, FDA | |
| Recombinant DNA | | |
| psPAX2 | Addgene | Cat # 12260 |
| pcDNA-SARS-COV-2 Spike | Liu et al., 2021 | |
| pTRIP-luc | Liu et al., 2010 | |
| RNAscope Reagents | | |
| RNAscope 2.5 HD RED kit | ACD | Cat #322373 |
| V-nCoV2019-orf1ab | ACD | Cat #895661 |
| Mm PPIB probe (positive control) | ACD | Cat #313911 |
| dapB probe (negative control) | ACD | Cat #310043 |
| Software and Algorithms | | |
| Prism 9.0 software | GraphPad | |
| SnapGene | GSL Biotech LLC | |

### Cells and Viruses

Vero E6 cell line (Cat #CRL-1586) was purchased from American Type Cell Collection (ATCC) and cultured in eagle's minimal essential medium (MEM) supplemented with 10% fetal bovine serum (Invitrogen) and 1% penicillin/streptomycin and L-glutamine. A549-hACE2 (NR-53821) cells were obtained from BEI Resources and maintained in DMEM supplemented with 5% penicillin and streptomycin, and 10% fetal bovine serum (FBS) at 37° C. with 5% CO₂.

EpiAirway cells (AIR-100-HCF) and culturing media were purchased from MatTek. EpiAirway is a ready-to-use, 3D mucociliary tissue model consisting of normal, human-derived tracheal/bronchial epithelial cells cultured at the air-liquid interface (ALI). Cells were cultured in MatTek proprietary media for 2 days prior to usage. Mucus was washed off at the time of infection.

The SARS-CoV-2 isolate WA1/2020 (NR-52281, lot 70033175) was obtained from BEI Resources, NIAID, NIH, and had been passed three times on Vero cells and one time on Vero E6 cells prior to acquisition. It was further passed once on Vero E6 cells before use. The virus has been sequenced and verified to contain no mutations compared to its original seed virus.

### Production of SARS-CoV-2 Recombinant Virus

SARS-CoV-2 recombinant virus was generated using a 7-plasmid reverse genetic system based on the virus strain (2019-nCoV/USA_WA1/2020) isolated from the first reported SARS-CoV-2 case in the U.S. (Xie et al., Cell Host Microbe 27:841-848, 2020). Fragment 4 was subsequently subcloned into a low-copy plasmid pSMART LCAmp (Lucigen) to increase stability. To introduce Nsp1^{N128S/K129E} and K164A/H165A mutations, pUC57-CoV2-F1 plasmids containing mutated Nsp1 were first created by using overlap PCR method with the following primers:

```
M13F:
  (SEQ ID NO: 19)
gtaaaacgacggccagt
N128S/K129Ef:
  (SEQ ID NO: 20)
taagaacggtAGTGAGggagctggtggccatagtta
N128S/K129E r:
  (SEQ ID NO: 21)
caccagctccCTCACTaccgttcttacgaagaagaa
K164A/H165Af:
  (SEQ ID NO: 22)
aaaactggaacactGCcGCcagcagtggtgttacccgtga
K164A/H165Ar:
  (SEQ ID NO: 23)
gggtaacaccactgctgGCgGCagtgttccagttttcttgaa
NheIr:
(SEQ ID NO: 24)
cacgagcagcctctgatgca
```

PCR fragments were digested with Bgl II/Nhe I and ligated into Bgl II/Nhe I digested F1 plasmid. The spike ΔPRRA mutation was introduced into pUC57-CoV2-F6 using overlap PCR with the following primers:

```
M13F:
  (SEQ ID NO: 25)
gtaaaacgacggccagt
ΔPRRA-f:
  (SEQ ID NO: 26)
actcagactaattctcgtagtgtagctagtcaatc
ΔPRRA-r:
  (SEQ ID NO: 27)
actagctacactacgagaattagtctgagtctgat
BglIIr:
(SEQ ID NO: 28)
cagcatctgcaagtgtcact
```

PCR fragments were digested by Kpn I/Bgl II and ligated into Kpn I/Bgl II digested F6 plasmid. To delete the ORF6-ORF8 region, an overlap PCR was performed using the following primers:

```
Mf:
  (SEQ ID NO: 29)
ttaattttagccatggcaga
ORF68f:
  (SEQ ID NO: 30)
tttgcttgtacagtaaacgaacaaactaaaatgtc
ORF68r:
  (SEQ ID NO: 31)
ttttagtttgttcgtttactgtacaagcaaagcaa
AvrIIr:
(SEQ ID NO: 32)
gaagtccagcttctggccca
```

PCR fragments were digested by Mlu I/Avr II and ligated into Mlu IAvr II digested pCC1-CoV-2-F7 plasmid. The resultant plasmids were validated by restriction enzyme digestion and Sanger sequencing.

In vitro transcription and electroporation were carried out following procedures that were detailed elsewhere (Xie et al., Nat Protoc 16:1761-1784, 2021). To recover the virus, the RNA transcript was electroporated into Vero E6 cells. Virus after passage 1 was titrated by plaque forming assay in Vero E6 cells and verified by deep sequencing.

### Hamster Challenge Experiments

Adult male outbred Syrian hamsters were purchased from Envigo and held at the FDA vivarium. All experiments were performed within a biosafety level 3 (BSL-3) suite. The animals were implanted subcutaneously with IPTT-300 transponders (BMDS), randomized, and housed two per cage in sealed, individually ventilated rat cages (Allentown). Hamsters were fed irradiated 5P76 (Lab Diet) ad lib, housed on autoclaved aspen chip bedding with reverse osmosis-treated water provided in bottles, and all animals were acclimatized at the BSL3 facility for 4-6 days or more prior to the experiments.

For challenge studies, adult (6-12 months old) Syrian hamsters were anesthetized with 3-5% isoflurane following procedures as described previously (Selvaraj et al., Life Sci Alliance 4(4):e202000886, 2021; Stauft et al., Virology 556:96-100, 2021). Intranasal inoculation was done by pipetting 10 PFU or desirable doses of SARS-CoV-2 in 50 μl volume dropwise into the nostrils of the hamster under anesthesia. Following infection, hamsters were monitored daily for clinical signs and weight loss. Nasal wash samples taken on days 1-, 2-, 3- and 4-day post infection to test for sgRNA by RT-qPCR and infectious virus by TCID₅₀ in Vero E6 cells. Nasal washes were collected by pipetting −160 μl sterile phosphate buffered saline into one nostril when hamsters were anesthetized by 3-5% isoflurane. For tissue collection, a subset of hamsters was humanely euthanized by intraperitoneal injection of pentobarbital at 200 mg/kg and lungs were processed for histopathology. Blood collection was performed under anesthesia (3-5% isoflurane) through gingival vein puncture or cardiac puncture when animals were euthanized.

### Mouse Infection Experiments

Adult K18-hACE2 mice were purchased from the Jackson laboratory and held at FDA vivarium. All experiments were performed within a biosafety level 3 (BSL-3) suite. For infection studies, mice were first anesthetized by 3-5% isoflurane. Intranasal inoculation was done by pipetting 10⁵ PFUSARS-CoV-2 in 50 μl volume dropwise into the nostrils of the mouse. Mice were weighed and observed daily. For tissue collections, mice were euthanized by CO₂ overdose on days 2, 4, 6 as necessary.

### Sars-CoV-2 Pseudovirus Production and Neutralization Assay

Procedures as described previously (Selvaraj et al., Life Sci Alliance 4(4):e202000886, 2021; Stauft et al., Virology 556:96-100, 2021).

RNA Isolation and qRT-PCR

Procedures as described previously (Selvaraj et al., Life Sci Alliance 4(4):e202000886, 2021; Stauft et al., Virology 556:96-100, 2021).

### RNAseq

To prepare sequencing libraries, RNA was first extracted using the Trizol-chloroform method from the lung homogenates and nasal turbinates. The aqueous portion was further purified using RNeasy mini kit (Qiagen, Gaithersburg, MD). RNA quality was assessed using Agilent 2100 Bioanalyzer (Agilent Technologies, Santa Clara, CA), and the RNA integration numbers (RIN) were all greater than 9. An aliquot (1 g) of each sample of total RNA was used to prepare sequencing libraries using Illumina Stranded Messenger RNA Prep (ligation based). The cDNA libraries were normalized and loaded onto a NovaSeq 6000 sequencer (Illumina, San Diego, CA) for deep sequencing of paired end reads of 2×100 cycles. The sequencing reads for each sample were mapped to the respective reference genomes of Mesocricetus auratus (BCM_Maur_2.0) by Tophat (v2.1.2). Cufflinks (v2.2.1) was then used to assemble transcripts, estimate abundances and test for differential expression. The sequencing and initial data analysis using Qiagen CLC Genomics Workbench (version 21) was performed by FDA Next Generation Sequencing Core Facility. The sequencing and initial data analysis using Qiagen CLC Genomics Workbench (version 21) was performed by FDA Next Generation Sequencing Core Facility. Raw and processed data have been deposited to NCBI (GEO accession number GSE199922s).

Further data analysis was done using R Studio 1.4.1106 (R-project.org). Heatmaps were constructed using heatinap library. The gene list for signaling pathways were obtained from hallmark gene sets in Molecular Signatures Database (MSigDB) (Subramanian et al., Proc Nal Acad Sci USA 102, 15545-15550, 2005). The figures were assembled in Adobe Photoshop. This work utilized the computational resources of the NIH HPC Biowulf cluster (hpc.nih.gov).

### Histopathology Analyses

Procedures as described previously (Selvaraj et al., Life Sci Alliance 4(4):e202000886, 2021; Stauft et al., Virology 556:96-100, 2021). Tissues (hearts, brains, lungs, trachea, and nasal turbinates) were fixed in 10% neural buffered formalin overnight and then processed for paraffin embedding. The 5-μm sections were stained with hematoxylin and eosin for histopathological examinations. Images were scanned using an Aperio ImageScope. Blinded samples were graded by a licensed pathologist for the following twelve categories: consolidation, alveolar wall thickening, alveolar airway infiltrates, perivascular infiltrates, perivascular edema, type 11 pneumocyte hyperplasia, atypical pneumocyte hyperplasia, bronchiole mucosal hyperplasia, bronchiole airway infiltrates, proteinaceous fluid, hemorrhage, vasculitis. Grading: 0=none, 1=mild, 2=moderate, 3=severe. A graph was prepared by summing up the score in each category.

### In-Situ Hybridization (RNAscope)

To detect SARS-CoV-2 genomic RNA in formalin-fixed paraffin-embedded (FFPE) tissues, ISH was performed using the RNAscope 2.5 HD RED kit, a single plex assay method (Advanced Cell Diagnostics; Catalog 322373) according to the manufacturer's instructions. Briefly, Mm PPIB probe detecting peptidylprolyl isomerase B gene (housekeeping gene) (catalog 313911, positive-control RNA probe), dapB probe detecting dihydrodipicolinate reductase gene from Bacillus subtilis strain SMY (a soil bacterium) (catalog 310043, negative-control RNA probe) and V-nCoV2019-orflab (catalog 895661) targeting SARS-CoV-2 positive-sense (genomic) RNA. Tissue sections were deparaffinized with xylene, underwent a series of ethanol washes and peroxidase blocking, and were then heated in kit-provided antigen retrieval buffer and digested by kit-provided proteinase. Sections were exposed to ISH target probes and incubated at 40° C. in a hybridization oven for 2 hours. After rinsing, ISH signal was amplified using kit-provided pre-amplifier and amplifier conjugated to alkaline phosphatase and incubated with a fast-red substrate solution for 10 minutes at room temperature. Sections were then stained with 50% hematoxylin solution followed by 0.02% ammonium water treatment, dried in a 60° C. dry oven, mounted, and stored at 4° C. until image analysis.

### Lung Immunofluorescence Analyses

FFPE lung sections 4 μm thick were dewaxed, rehydrated, and heat-treated in a microwave oven for 15 minutes in 10 mM Tris/1 mM EDTA buffer (pH 9.0). After cooling for 30 min at room temperature, heat-retrieved sections were blocked in PBST with 2.5% bovine serum albumin (BSA) for 30 minutes at room temperature (RT) followed by overnight incubation at 4° C. with primary antibodies in 1% BSA. Primary antibodies used included SARS nucleocapsid protein (Sino Biologicals, 40143-MM05), prosurfactant protein C (EMD Millipore, AB3786), Iba1 (Abcam, ab5076), and RAGE (Abcam, ab216329). Sections were rinsed and incubated with Alexa Fluor 488 and Alexa Fluor 647-conjugated secondary antibodies for 1 hour at RT (ThermoFisher, Waltham, MA). Nuclei were counterstained with Hoechst 33342. For double labeling experiments, primary antibodies were mixed and incubated overnight at 4° C. For negative controls, sections were incubated without the primary antibody or mouse and rabbit isotype antibody controls. Sections stained with conjugated secondary antibodies alone showed no specific staining. Whole slide fluorescence imaging was performed using a Hamamatsu NanoZoomer 2.0-RS whole-slide digital scanner equipped with a 20× objective and a fluorescence module #L11600. Analysis software NDP.view2 was used for image processing (Hamamatsu Photonics, Japan).

### TCID₅₀

Procedures as described previously (Selvaraj et al., Life Sci Alliance 4(4):e202000886, 2021; Stauft et al., Virology 556:96-100, 2021).

### Plaque Assay

Nasal wash samples were 10-fold serially diluted and added to a 24-well plate containing freshly confluent Vero E6 cells. For tissue samples, entire trachea or nasal turbinates or the left lobe of the lung (−0.2 gram) were resuspended in 1 milliliter MEM and homogenized on a Precellys Evolution tissue homogenizer with a Cooling Unit (Bertin). Tissue homogenates were then 10-fold serially diluted and added to Vero E6. After 1 h, the mixture was removed and replenished with Tragacanth gum overlay (final concentration 0.3%). Cells were incubated at 37° C. and 5% CO₂ for 2 days, then fixed with 4% paraformaldehyde (PFA), followed by staining of cells with 0.1% crystal violet in 20% methanol for 5-10 minutes. The infectious titers were then calculated and plotted as plaque forming units per milliliter (PFU/ml).

### Measurement of Antibody by ELISA

SARS-CoV-2 S and RBD antigens for ELISA were prepared in a baculovirus expression system using procedures as published elsewhere (Meseda et al., NPJ Vaccines 6:145, 2021).

### Statistical Analysis

One-way ANOVA was used to calculate statistical significance through GraphPad Prism (9.1.2) software for Windows, GraphPad Software, San Diego, California USA.

### Example 2: Rational Attenuation of SARS-CoV-2

Although a single mutation may attenuate a virus, a LAV that differs from the wild-type virus by only one or two mutations poses an inherent safety concern due to the possibility of reversion. For that reason, the following modifications were made to the ancestral WA1/2020 viral genorne: First, the polybasic insert (PRUA immediately upstream of the furin cleavage site was removed from the virus. Such a modification abolishes the S I/S2 cleavage of the spike protein and significantly reduces infection of the lung (Johnson et al., Nature 591:293-299, 2021; Liu et al., J Virol 95(11):e01751-20, 2021). Second, known IFN antagonists, ORFs6-8, were deleted from the viral genome. Third, a pair of mutations (K164A/H16.5A) that significantly reduce cytotoxicity of SARS-CoV-2 (Liu et al., J Virol 96(6):e0221621, 2022) was introduced into the C-terminus of Nsp1. The three-pronged genetic modifications are to decrease infection of the lung, reduce inflammation and interferon antagonism, and alleviate Nsp-i-mediated toxicity. Ultimately, a recombinant virus termed “WA1-ΔPRRA-ΔORF6-8-Nsp1^{K64A/H165A}” was obtained. For comparison purpose, also generated were two other recombinant viruses, WA1-ΔPRRA and WA1-ΔPRRA-ΔORF6-8-Nsp1^{N128S/K129E}, respectively (FIG. 1A). WA1-ΔPRRA only has the polybasic insert removed, whereas WA1-ΔPRRA-ΔORF6-8-Nsp1^{N1285/KJ29E} has both the polybasic insert and ORFs6-8 deleted and then contains a pair of mutations (N128S/K129E) that did not efficiently abolish Nsp1-mediated cytotoxicity as did K164A/H165A (Liu et al., J Virol 96(6):e0221621, 20221 All recombinant viruses formed plaques in Vero E6 cells and reached titers of 10⁷ pfu/ml (FTG. 1B). For brevity, the three recombinant viruses are also referenced as “ΔPRRA”, “Nsp1-K164A1H165A”, and “Nsp1-N128S/K129E” throughout the text. To monitor the genome stability, recombinant viruses were passaged five times in Vero E6 cells and then deep sequenced to confirm identity. Sanger sequencing was also performed to detect the presence of K164A/H165A in Nsp1 (FIG. 1C). Overall, no reversion was found, and the genome of recombinant viruses appeared to be stable after cell passages. The three recombinant viruses grew with similar kinetics in A549-hACE2 cells to comparable titers (FIG. 1D) Compared to the ancestral virus (WA1/2020), all three recombinant viruses poorly infected primary human airway cells that were cultured at the air-liquid interface, with Nsp1-K164A/H165A displaying the lowest infectivity (FIG. 1E).

### Example 3: Attenuation of Nsp1-K164a/H165A in K18-hACE2 Transgenic Mice

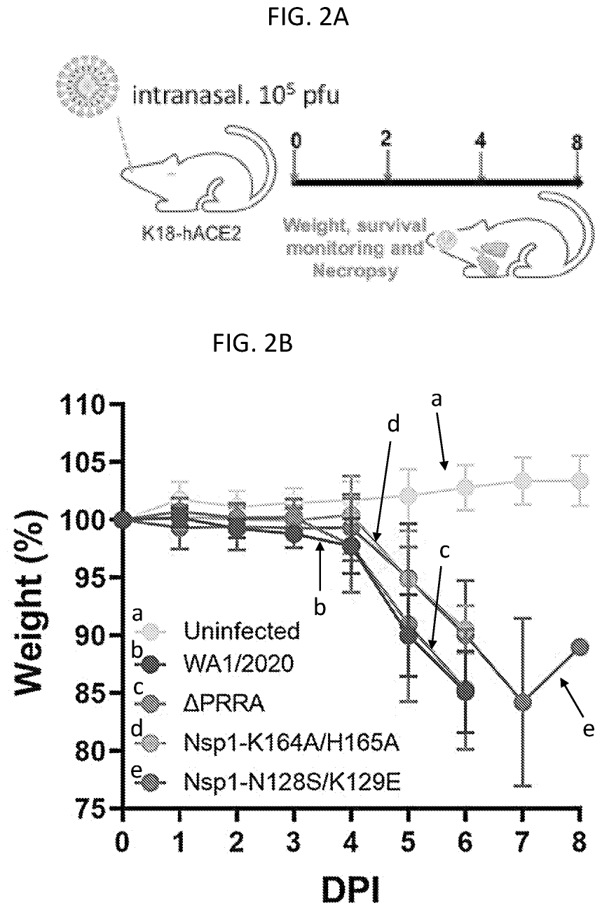

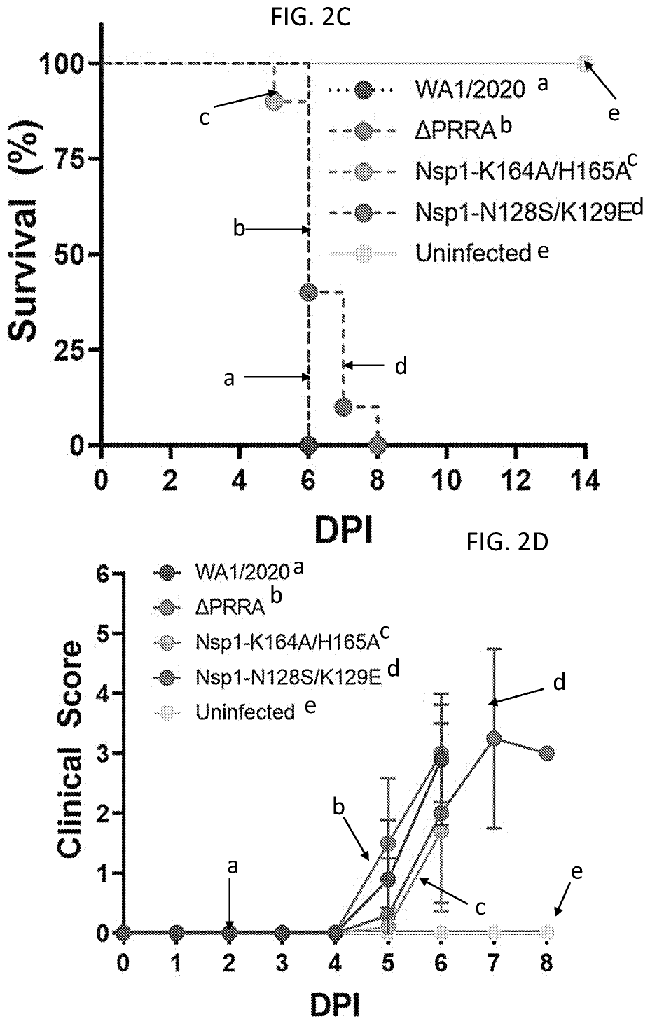

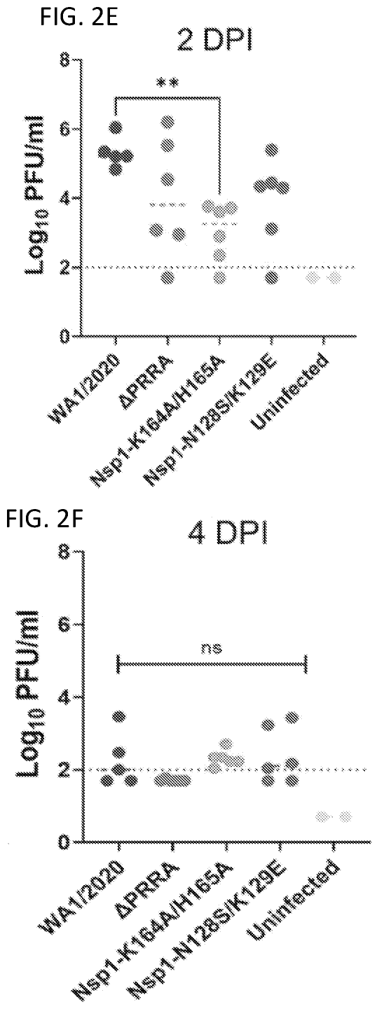

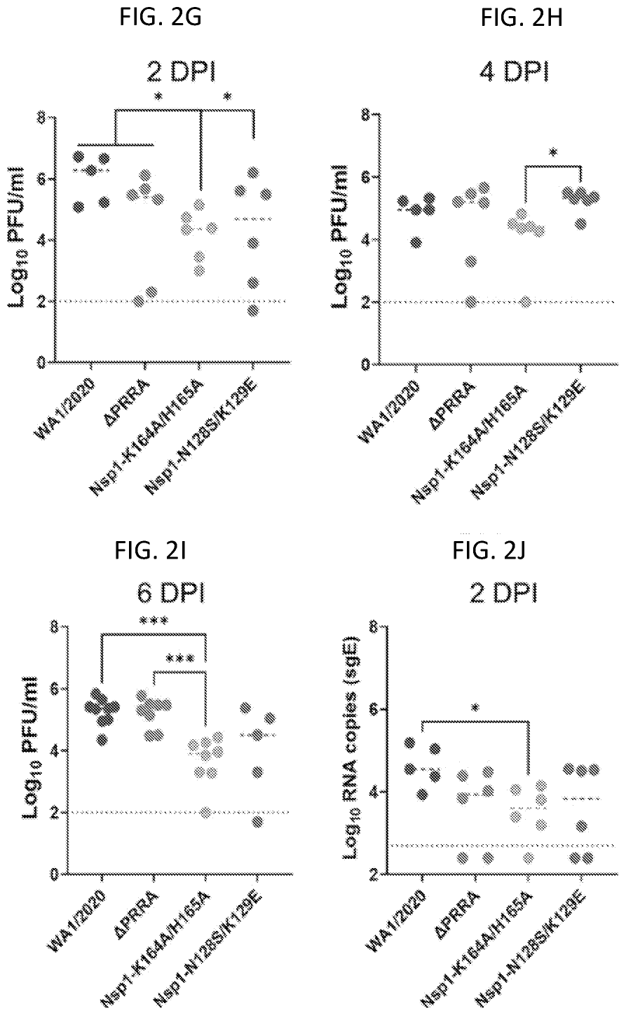

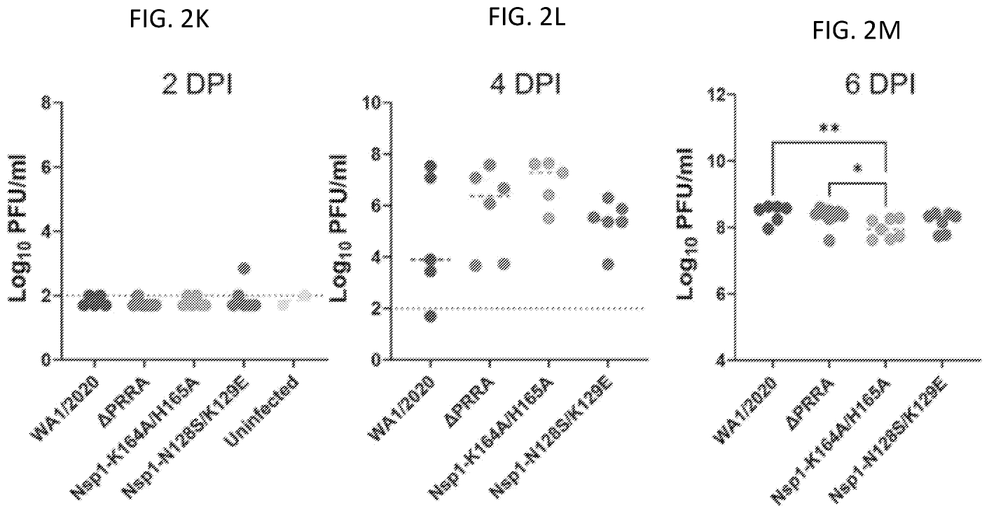

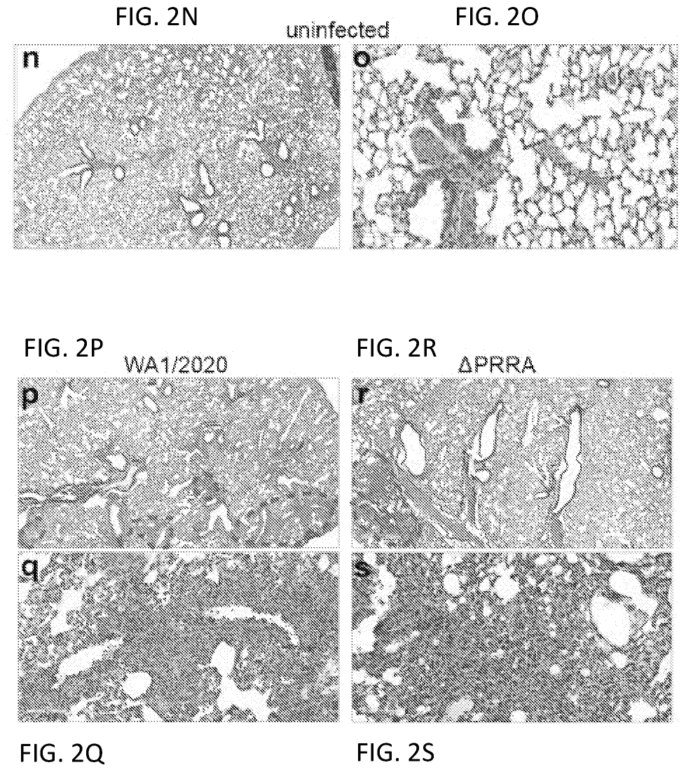

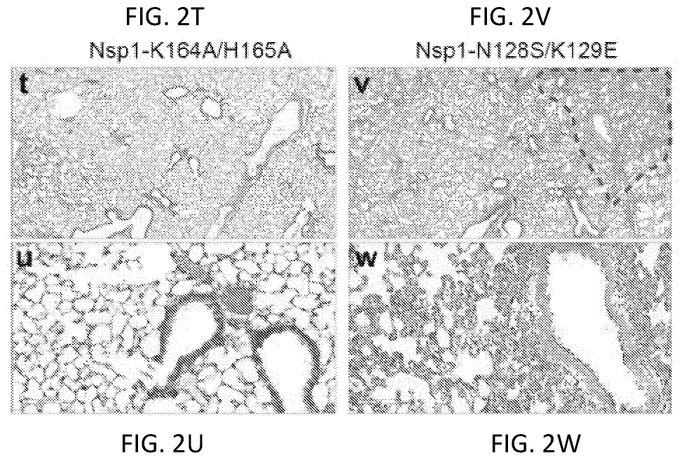

To test attenuation in vivo, adult K18-hACE2 transgenic mice were divided into five groups (n=10/group) and intranasally inoculated with 10⁵ plaque forming unit (PFU) of WA1/2020, ΔPRRA, Nsp1-K164A/H165A, or Nsp1-N128S/K129E, or left uninoculated. Weight, survival, and clinical signs of illness were monitored for eight days. All infected mice succumbed to the infection by day 8 (FIGS. 2A-2D). Because encephalitis following SARS-CoV-2 infection is known to cause lethality in this model (Winkler et al., Nat Immunol 21:1327-1335, 2020; Oladunni et al., Nat Commun 11, 6122, 2020; Rathnasinghe et al., Emerg Microbes Infect 9: 2433-2445, 2020; Golden et al., JCI Insight 5(19):e142032, 2020; Yinda et al., PLoS Pathog 17(1):e1009195, 2021; Rathnasinghe et al., Emerg Microbes Infect 9(1):2433-2445, 2020; Bao et al., Nature 583:830-833, 2020), possible attenuation of the virus in the respiratory tract could have been masked by the lethality caused by encephalitis. To mitigate the limitations of the K18-hACE2 mouse model, the viral loads were quantified in nasal turbinates, lungs, and brains at 2, 4, 6 days post infection (DPI). In nasal turbinates, the median log₁₀-transformed infectious titers peaked by 2 DPI at 5.33, 4.01, 3.01 and 3.89 for WA1/2020, ΔPRRA, Nsp1-K164A/H165A, and Nsp1-NI28S/K129E infected mice, respectively. Infectious titers subsided to approximately the lower limit of quantification by 4 DPI (FIGS. 2E, 2F). In the lungs of Nsp1-K164A/H165A infected mice, infectious viral titers were ˜2 login lower compared to WA1/2020 infected animals at 2 and 6 DPI (FIGS. 2O-2I). The lung viral loads in Nsp1-K164A/H165A infected mice also trended lower than those from ΔPIRA and Nsp1-N128S/K129E groups, reaching statistical significance at 4 and 6 DPL The viral loads in the brain, however, were largely comparable among the four infected groups except that at 6 DPI the Nsp1-K164A/H165A group had the lowest viral titers (FIGS. 2K-2M). At 2 DPI, there was very little detectable infectious virus in the brain, but the viral load in the brains rose in delayed kinetics as opposed to that in the respiratory tract. High viral loads in the brains of the Nsp1-K164A/H165A group at 6 DPI were coupled with a lack of pathology in lung tissues. Haematoxylin and eosin (HE) staining (FIGS. 2N-2W) identified lung lesions in WA1/2020, ΔPRRA, and Nsp1-N128S/K129E infected mice. Generally, there were peribronchiolar and perivascular immune infiltration. By contrast, Nsp1-K164A/H165A infected lungs had less than 1% impacted area with little pathology, which are nearly indistinguishable from the uninfected mice. Altogether, these results demonstrated that the Nsp1-K164A/H165A virus was primarily attenuated in upper and lower respiratory tract in K18-hACE2 mice but was still neuroinvasive in this highly sensitive mouse model.

### Example 4: Attenuation of Nsp1-K164a/H165A in Syrian Hamsters

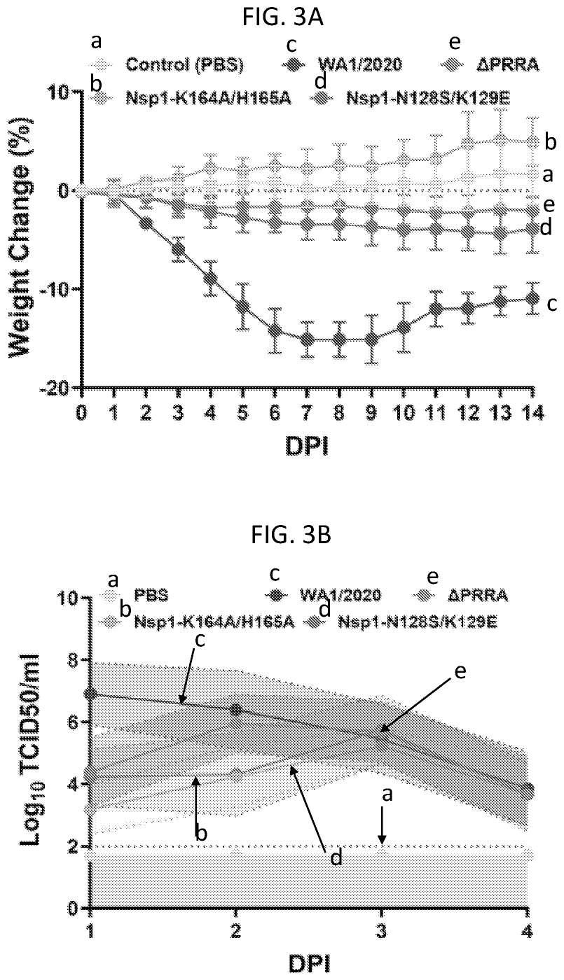

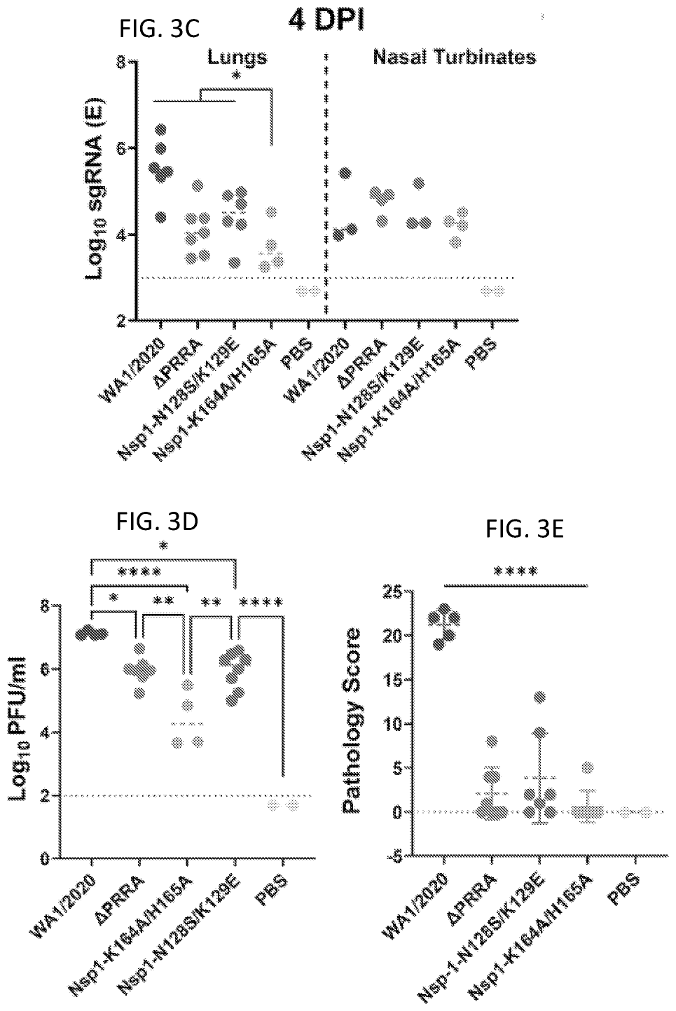

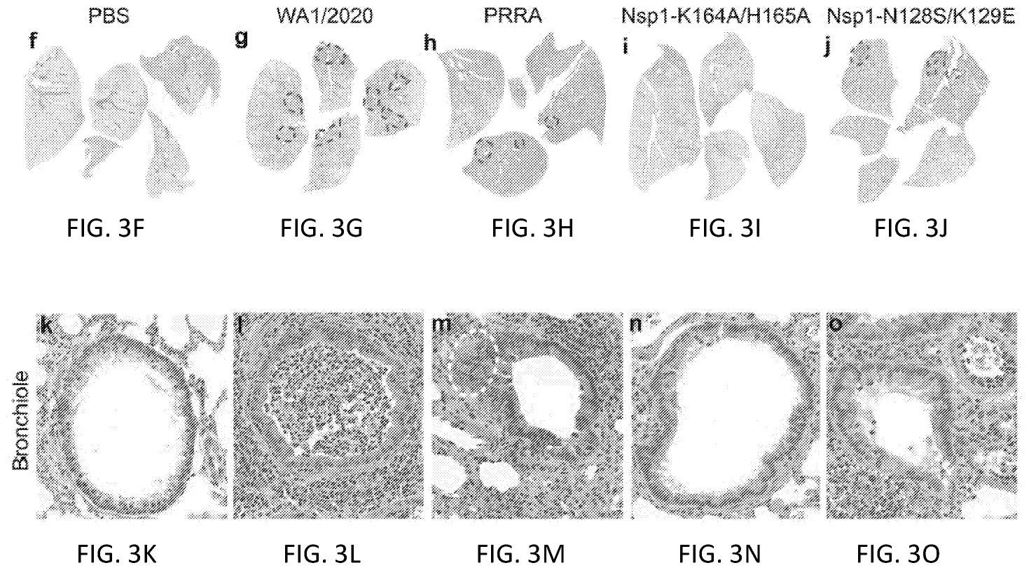

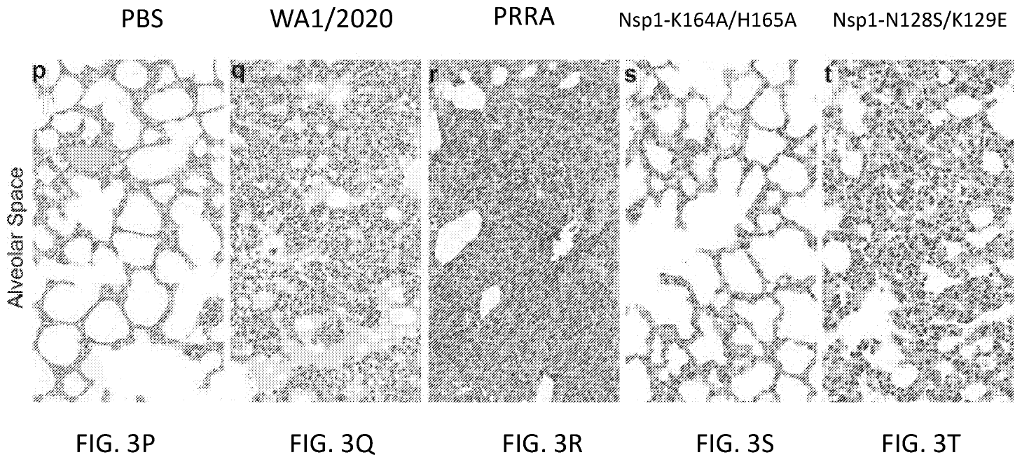

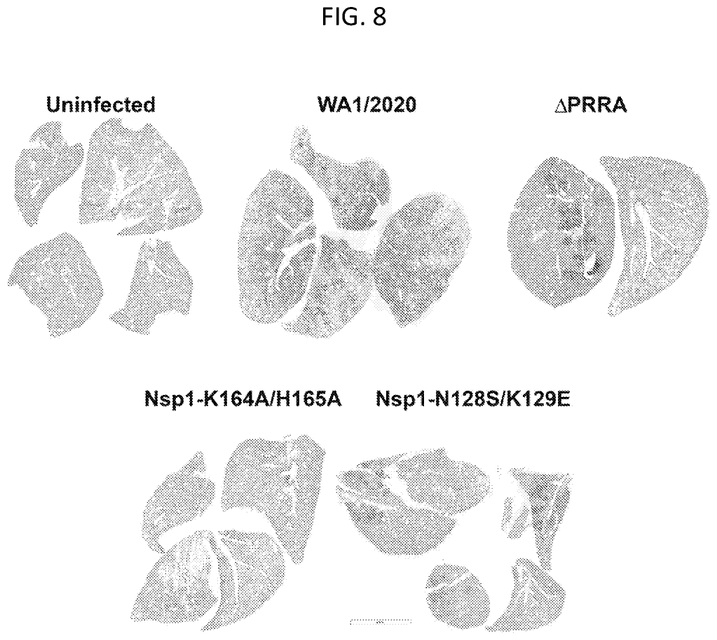

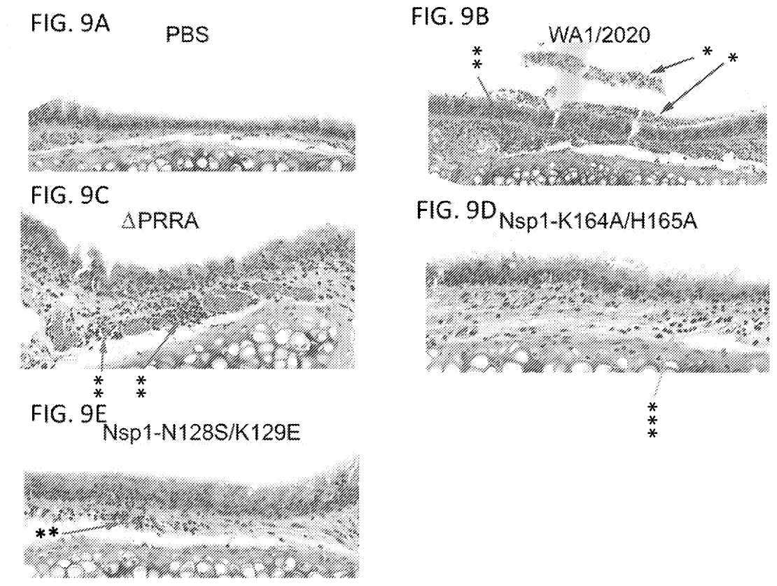

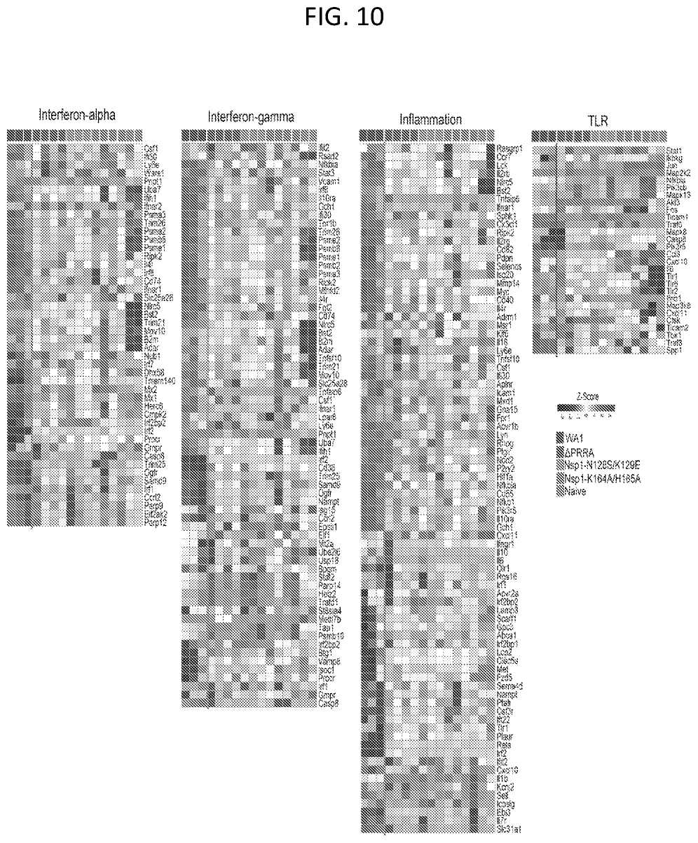

Syrian hamsters are highly susceptible to SARS-CoV-2 and have been widely used in COVID-19 research (Sia et al., Nature 583:834-838, 2020; Chan et al., Clin Infect Dis 71:2428-2446, 2020; Imai et al., Proc Natl Acad Sci USA 117:16587-16595, 2020). Given the intrinsic shortcomings of the K18-hACE2 mouse model due to fatal neuroinvasion, the possible attenuation of Nsp1-K164A/H165A was further evaluated in hamsters. To this end, five groups of 6-month-old Syrian hamsters were intranasally inoculated with 10⁴ PFU of each virus or left uninoculated. This inoculum has consistently yielded weight loss, clinical signs, and lung pathology in Syrian hamsters (Selvaraj et al., Life Sci Alliance 4(4):e202000886, 2021). Shown in FIG. 3A. WA1/2020 infected hamsters showed 18% weight loss on 7 DPI, whereas ΔPRRA, and Nsp1-N128S/K129E groups displayed no more than 5% weight loss over a period of 14 days. Nsp1-K164A/H1165A infected animals (n=8), like the uninfected group, had no weight loss at all through the study. During the first four days following infection, the log t-transformed infectious viral titers in nasal wash samples were measured by a TCID₅₀ assay. Shown in FIG. 3B, the infectious titers from Nsp1-K164A/H165A infected animals were about two log₁₀ lower than that of WA1/2020 infected hamsters at 1 and 2 DPI and trended lower than those from ΔPRRA and Nsp1-N1285/K129E-infected animals. Infectious nasal viral titers from all groups subsided to just above the limit of quantification at 4 DPI. Similarly, subgenomic RNA (sgRNA) titers in nasal turbinates from all groups were just above the limit of quantification at 4 DPI (FIG. 3C). Log₁₀-transformed sgRNA copies per ml in lung homogenates were 5.55, 4.0, 3.37, and 4.71, for WA1/2020, ΔPRRA, Nsp1-K164A/H165A, and Nsp1-N128S/K129E infected hamsters, respectively, at 4 DPI (FIG. 3C). Log₁₀-transformed infectious viral titers in lung homogenates were 7.13, 5.96, 4.42, and 5.95 for WA112020, ΔPRRA, Nsp1-K164A/H165A, and Nsp1-N128S/K129E infected hamsters, respectively (FIG. 3D). RNAscope revealed that viral RNA was present only along the bronchial epithelium in Nsp1-K164A/H165A-infected hamsters and the amount of staining was much less than the other three infected groups (FIG. 8). Overall, viral loads of Nsp1-K164A/H165A-infected hamsters were 100- to 1000-fold lower than WA1/2020-infected animals and were also noticeably lower than ΔPRRA and Nsp-NI128S/K129E-infected animals. The lung pathology was subsequently scored. Once again, Nsp1-K164A/H165A-infected hamsters had minimal histopathological changes in the lung at 4 DPI (FIG. 3E). By contrast, WA1/2020 infection induced massive peribronchiolar edema and perivascular immune cell infiltrates, which led to significant consolidation (FIGS. 3F-3T). ΔPRRA and Nsp1-N128S/K129E groups occasionally had areas where type II hyperplasia and immune infiltration were found. Nsp1-K164A1H165A infected hamsters showed minimal pathological changes in the lung, sometimes indistinguishable from uninfected animals. The pathology of trachea followed the same trend observed in the lung with Nsp1-K164A/H165A infected animals showing only minimal submucosal lymphoplasmacytic infiltrates (FIGS. 9A-9E). None of the infections led to noticeable changes in heart and other critical organs as previously reported (Selvaraj et al., Life Sci Alliance 4(4):e202000886, 2021).

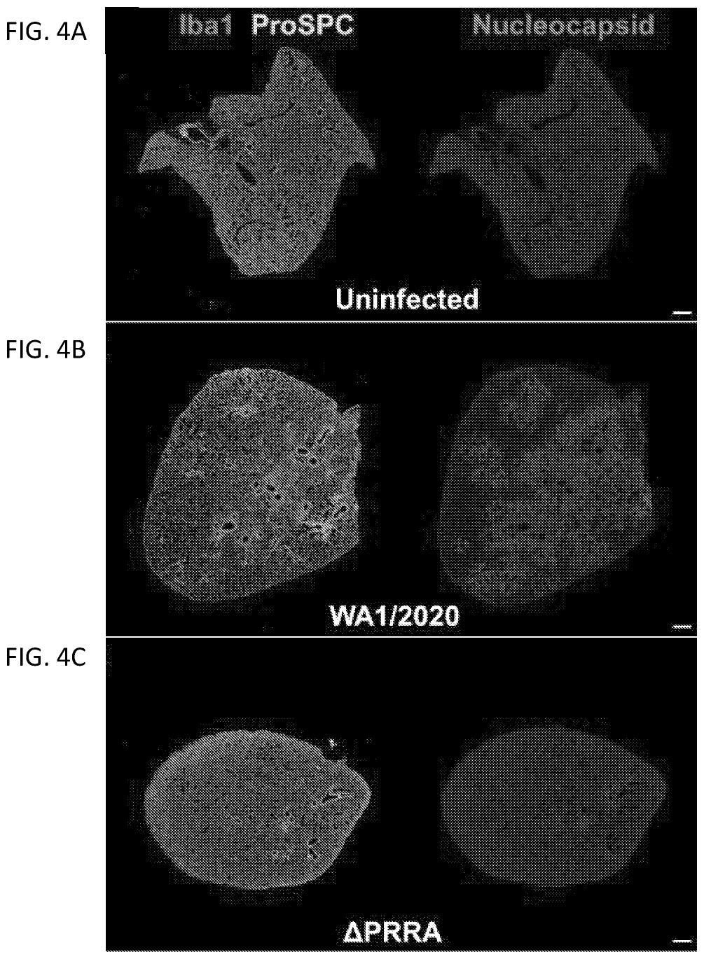

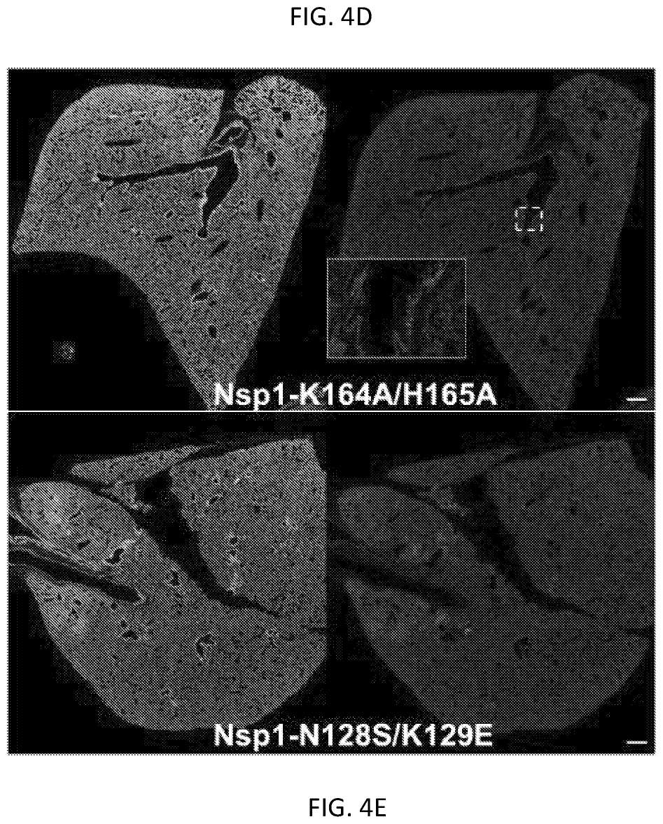

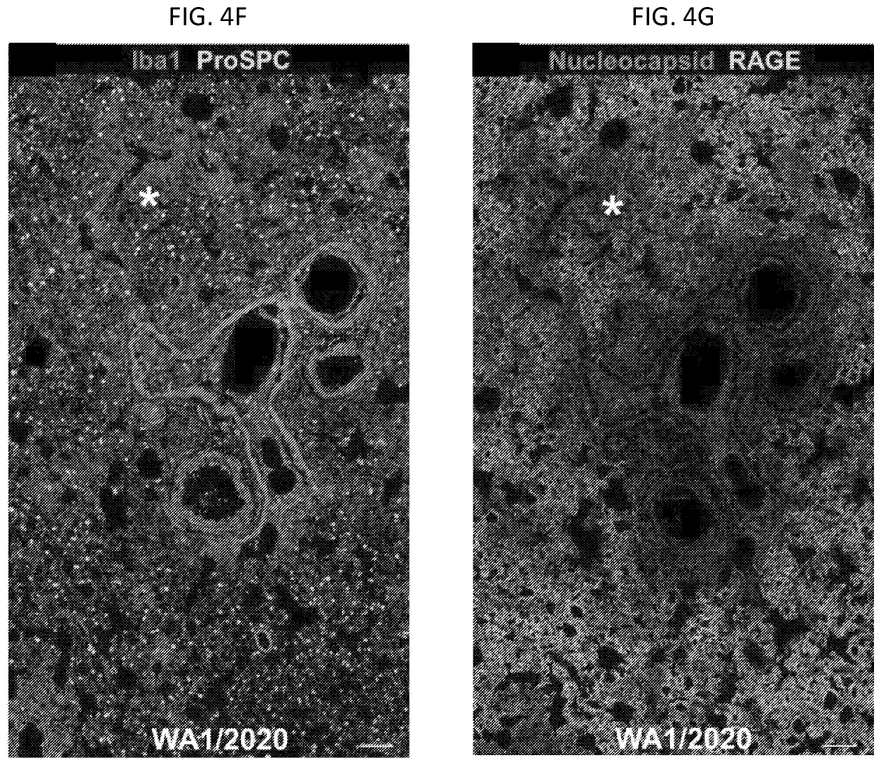

Immunofluorescence analyses showed that regions of consolidation in WA1/2020 infected lungs were dominated by Iba1-expressing macrophages (FIG. 4). Consolidated Iba1 staining was not detected or minimal in uninfected. ΔPRRA and Nsp1-K164A/11165A groups while consolidated Iba1 regions were visible in Nsp1-N128S/K129E-infected animals. Prominent staining for viral nucleocapsid was present in alveolar epithelium surrounding consolidated regions and within affected bronchioles in WA1/2020 (FIGS. 4A, 4B). Nucleocapsid staining was limited to bronchiolar epithelium in Nsp1-K164A/H165A group (FIG. 4A) but reached alveolar epithelium in ΔPRRA and Nsp1-N128S/K129E infected animals. Reduced staining for RAGE and ProSPC, markers of type I and type 2 epithelial cells, respectively, highlighted the excessive epithelial damage in regions of consolidation (FIGS. 4F, 4G).

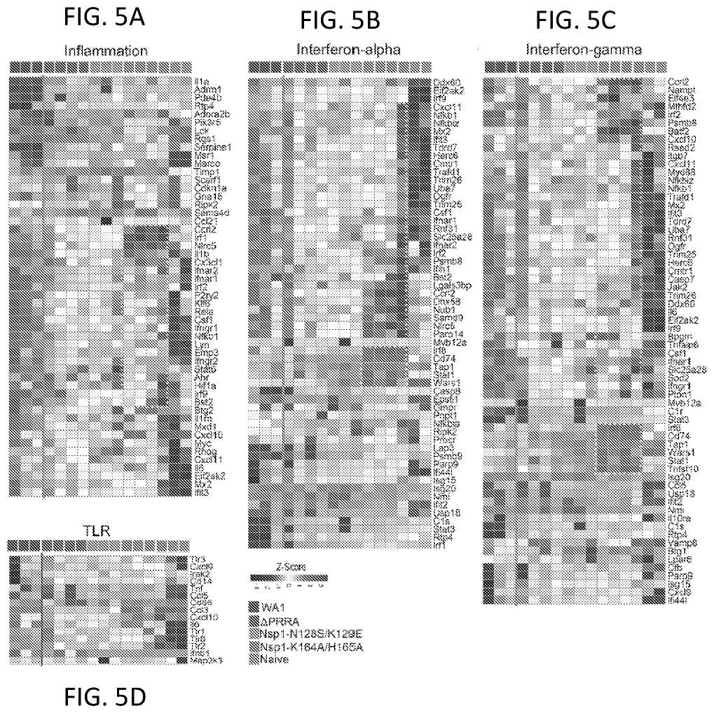

To further assess changes at the molecular level, RNA was isolated from both nasal turbinates and lung homogenates at 4 DPI and RNAseg analyses was performed. In nasal turbinates, comparison between WA1/2020 and ΔPRRA, Nsp1-N1285/K129E and Nsp1-K164A/4165A infected animals showed that WA1/2020 upregulated 34 and downregulated 17 genes in pathways of inflammation, upregulated 33 and downregulated 25 genes in pathways of type I IFN responses, and upregulated 39 and downregulated 8 genes in pathways of type 11 TFN responses (FIGS. 5A-5D). Most noticeably, Nsp1-K164A/H165A infection had the least effects on the expressions of proinflammatory markers, such as Mx2, lfit3, Tir6, Cxcxl10 and Nfkb1 (FIG. 5A). Nsp1-K164A/H165A infection upregulated the least numbers of genes of the interferon-alpha and gamma responses (FIGS. 5B, 5C), presumably due to the lowest viral load among all tested groups. In some cases, gene expression profiles of Nsp1-K164A/H-165A-infected hamsters were indistinguishable from those of uninfected hamsters. Strikingly, Nsp1-K164A/H165A specifically upregulated genes like Irf8, Tap1 and Stat1, all of which are important for antiviral defense (FIG. 5B). In the lungs, WA1-2020 induced 50 proinflammatory genes, 14 TLR signalling genes, 25 genes of the type I IFN and 37 genes of type II IFN pathways to higher levels than in ΔPRRA, Nsp1-K164A/H165A, and Nsp1-N128S/K129E infected hamsters. For those genes that were significantly downregulated in WA1/2020 infected animals, their expressions were restored in ΔPRRA, Nsp1-K164A/H165A, and Nsp1-N1285/K129E infected hamsters (FIG. 10). Notably, significant differences in terms of gene expression profiles were not observed in the lungs between ΔPRRA, Nsp1-K164A/H165A, and Nsp1-N128S/K129E infected animals, which may be a result of limited numbers of samples or an overall very low viral loads in the lungs.

### Example 5: Intranasal Immunization of Syrian Hamsters with Nsp1-K164a/H165A Induced Potent Humoral Response and Protected Against WA1/2020 Challenge

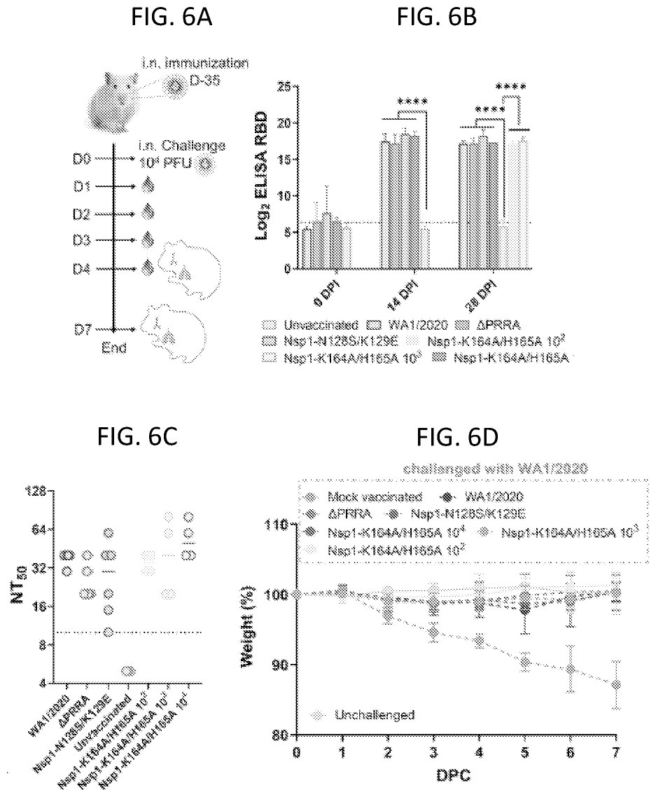

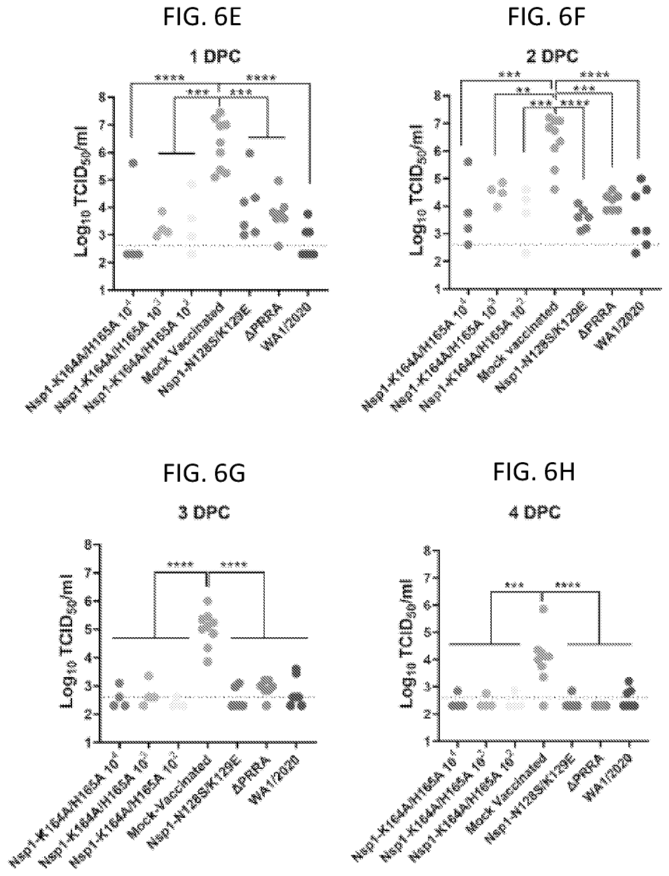

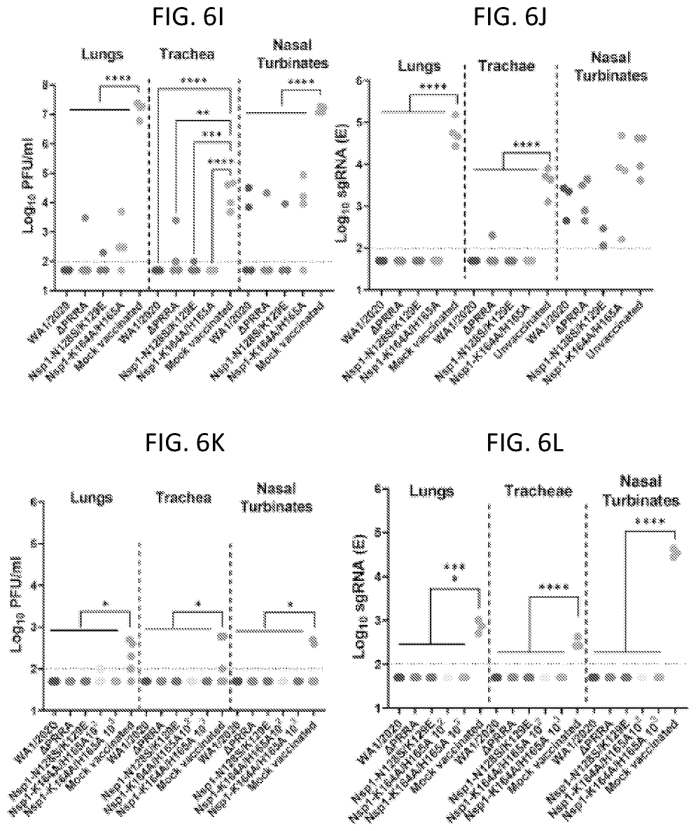

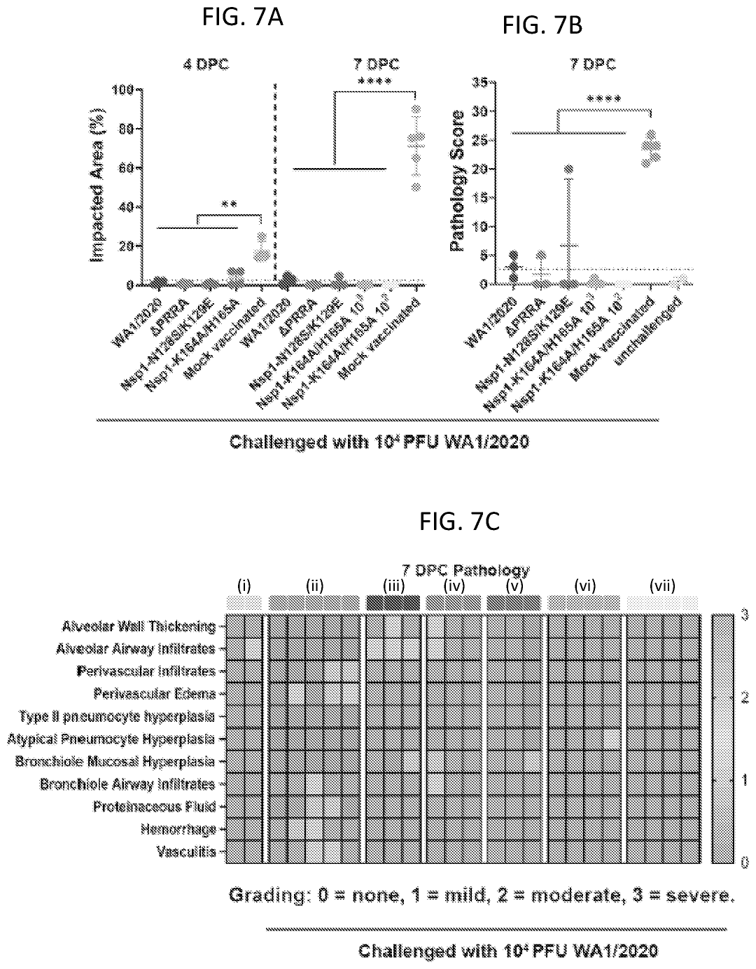

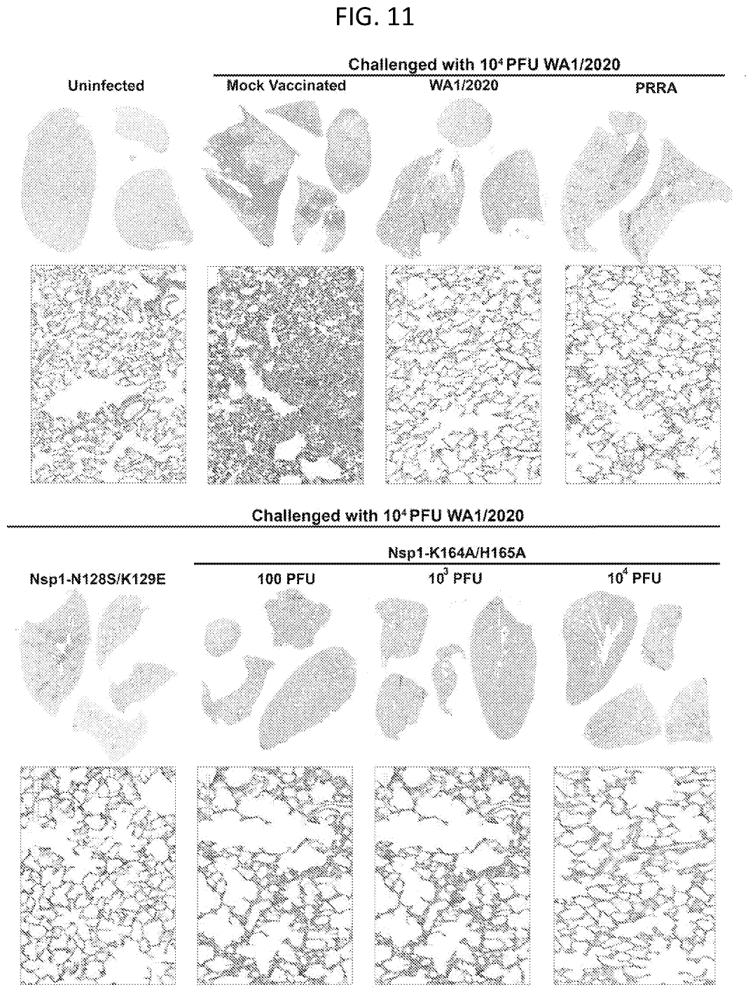

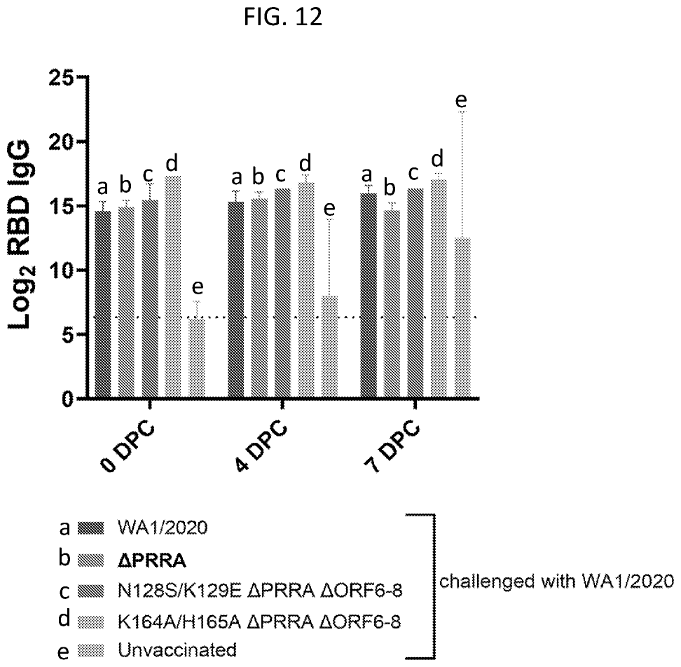

Based on the data obtained from Examples 2-4, Nsp1-K164A/H165A was the most attenuated recombinant virus and hence was chosen for subsequent evaluation of immunogenicity and efficacy as a LAV candidate. Adult Syrian hamsters were intranasally immunized with 10², 10³, and 10⁴ PFU Nsp1-K164A/H165A. For comparison, hamsters were also inoculated with 10′ PFU WA1/2020, ΔPRRA, and Nsp1-N128S/K129E and animals were housed until convalescence (FIG. 6A). A single dose of Nsp1-K164A/H165A induced binding and neutralizing antibodies (FIGS. 6B, 6C) to levels that are comparable to those from WA1/2020-infected hamsters at 14 or 28 days after immunization. A dose of 100 PFU was just as potent as a dose of 10′ PFU. Immunized and convalescent hamsters were subsequently challenged with 10⁴ PFU WA1/2020 virus and monitored for 7 days before necropsy. Mock vaccinated hamsters lost more than 15% body weight by day 7 post-challenge (DPC), whereas immunized hamsters from all three dosage groups did not lose any weight (FIG. 6D). At 1- and 2-days post-challenge, infectious viral titers in nasal wash samples collected from immunized animals were 3 to 4 log₁₀ lower than those from unvaccinated but challenged animals (FIGS. 6E, 6F) and largely resolved by 4 DPC (FIGS. 6G, 6H). Infectious viral titers and sgRNA titers in trachea and lungs were frequently below the limit of quantification in many of the immunized and convalescent hamsters at 4 and 7 DPC (FIGS. 6I-6L). Viral loads in the nasal turbinates of the immunized and convalescent hamsters were at least 4 logs lower at 4 DPC and then went undetectable at 7 DPC. Lastly, single-dose immunization with Nsp1-K164A/H165A completely protected hamsters from developing pneumonia upon challenge, with nearly 0% consolidation and no histopathological changes at 4 and 7 DPC (FIGS. 7A-7C and FIG. 11). An anamnestic antibody response was not observed in the Nsp1-K164A/H165A-vaccinated hamsters (FIG. 12), likely reflecting the robust protection and minimal viral replication in these animals, as found in convalescent animals (Selvaraj et al., Life Sci Alliance 4(4):e202000886, 2021).

### Example 6: Materials and Methods (Part 2)

This example describes the materials and experimental procedures for the studies described in Examples 7-12.

### Cells and Viruses

Vero E6 cell line (Cat #CRL-1586) was purchased from American Type Culture Collection (ATCC) and cultured in Dulbecco's minimal essential medium (MEM) supplemented with 10% fetal bovine serum (Invitrogen) and 1% penicillin/streptomycin and L-glutamine. Calu-3 cell line (Cat #HTB-55) was obtained from ATCC and maintained in EMEM+20% FBS. H1299-hACE2 is a human lung carcinoma cell line stably expressing human ACE2. The cell line was generated by lentiviral transduction of the NCI-1299 human lung carcinoma cell line (ATCC CRL-5803) with pLVX-hACE2 and selected with 1 μg/mL puromycin. A western blot was performed to confirm the expression of hACE2. H1299-hACE2 cells were maintained in DMEM supplemented with 5% penicillin and streptomycin, and 10% fetal bovine serum (FBS) at 37° C. with 5% CO₂.

The SARS-CoV-2 isolate WA1/2020 (NR-52281, lot 70033175) was obtained from BEI Resources, MAID, NIH, and had been passed three times on Vero cells and one time on Vero E6 cells prior to acquisition. It was further passed once on Vero E6 cells. SARS-CoV-2 hCoV-19/USA/MD-HP05647/2021 (Delta variant, Pango lineage B.1.617.2) was obtained from BEI resources, NIAID, NIH (NR-55672, Lot 70046635) and had been passaged once in Vero E6-TMPRSS2 and once in Calu-3 cells prior to acquisition. It was passaged once more in H1299-hACE2 cells to generate viral stocks. Passaged viruses were deep sequenced to confirm identity (100% match with the original sequence, free of tissue culture adaptive mutations such as the loss of the polybasic site between S1 and S2 subunit of the spike protein). SARS-CoV-2 isolates hCoV-19/USA/HI-CDC-4359259-001/2021 (B.1.1.529 Omicron, NR-56475), hCoV-19/USA/NY-MSHSPSP-PV56475/2022 (BA.2.12.1 Omicron, NR-56782), USA/MD-HP30386/2022 (BA.4, Omicron, NR-56802), and hCoV-19/USA/COR-22-063113/2022 (BA.5 Omicron, NR-58620) were obtained from BEI resources and used directly in experiments. Recombinant SARS-CoV-2 viruses were generated as described previously (Liu et al., Nat Commun 13:6792, 2022; Liu et al., Nat Comm 13(1):6792, 2022). The BA.1-LAV and BA.5 LAV were generated using standard molecular biology techniques. In brief, the WA1 spike sequence in the prototype Nsp1-K164A/H165A virus was replaced with corresponding BA.1 and BA.5 spike protein sequences. The polybasic inset “HRRA” was removed.

### Hamster Challenge Experiments

Adult male outbred Syrian hamsters were previously purchased from Envigo. All experiments were performed within a biosafety level 3 (BSL-3) suite. The animals were implanted subcutaneously with IPTT-300 transponders (BMDS), randomized, and housed 2 per cage in sealed, individually ventilated rat cages (Allentown). Hamsters were fed irradiated 5P76 (Lab Diet) ad lib, housed on autoclaved aspen chip bedding with reverse osmosis-treated water provided in bottles, and all animals were acclimatized at the BSL3 facility for 4-6 days or more prior to the experiments.

Adult male (5-6 months old) Syrian hamsters (Mesocricetus auratus) were anesthetized with (3-4% v/v) isoflurane and oxygen following procedures as described previously (Selvaraj et al., Life Sci Alliance 4(4):e202000886, 2021; Stauft et al., Virology 556:96-100, 2021; Jiron et al., JAm Assoc Lab Anim Sci 58:40-49, 2019). Intranasal inoculation was done by pipetting 10² PFU or 10⁴ PFUSARS-CoV-2 in 50 μl volume dropwise into the nostrils of the hamster under anesthesia. Following infection, hamsters were monitored daily for clinical signs and weight loss. Nasal washes were collected by pipetting −200 μl sterile phosphate buffered saline into one nostril when hamsters were anesthetized by 3-5% isoflurane. Nasal swabs were done as described previously (Langel et al., Sci Transl Med 14:eabn6868, 2022).

For airborne transmission, a subset (n=7) of hamsters inoculated with 10² PFU WA1/2020 or Nsp1-K164A/H165A were paired in divided cages to prevent direct contact to measure transmission to naive sentinels (Nunez et al., mSphere 6(3):e0050721, 2021). One hamster (WH363), paired with an actively shedding Nsp1-K164A/H165A vaccinated animal, did not show evidence of productive infection or seroconvert at 14 DPE and remained seronegative until just prior to BA2.12.1 challenge 4.5 months later. For these reasons, WH363 was removed from the challenge datasets.

For tissue collection, a subset of hamsters was humanely euthanized by intraperitoneal injection of pentobarbital at 200 mg/kg at 4 and 7 DPC. Lungs, trachea, and nasal turbinates were dissected for histopathology or homogenized for RNA extraction or titration in cell culture. Blood collection was performed under anesthesia (3-5% isoflurane) through gingival vein puncture or cardiac puncture when animals were euthanized. The left lobes of hamster lungs (−0.2 gram) were diced, divided, and resuspended in 1 milliliter MEM or TriZol reagent (RNA extraction) and homogenized on a Precellys Evolution tissue homogenizer with a Cooling Unit (Bertin). Trachea and nasal turbinates were homogenized the same way in TriZol Reagent. Splenocytes were extracted at 14 DPI from vaccinated and naive hamsters and IFNγ-secreting cells were identified after stimulation with spike and nucleocapsid antigen pools (BEI Catalog No. NR-52418 and NR-52419) by ELISpot (MABTECH, 3102-2H).

RNA Isolation and qRT-PCR

Procedures as described previously (Selvaraj et al., Life Sci Alliance 4(4):e202000886, 2021; Stauft et al., Virology 556:96-100, 2021). In brief, RNA was extracted from 0.1-gram tissue homogenates using QIAamp vRNA mini kit or the RNeasy 96 kit (QIAGEN) and eluted with 60 μl of water. Five μL RNA was used for each reaction in real-time RT-PCR. When graphing the results in Prism 9, values below the limit of quantification (LoQ) were arbitrarily set to half of the LoQ values. Unless otherwise specified, the unit for RNA copies are as presented as Log₁₀ RNA copes/μg tissue RNA.

### Histopathology Analyses

Procedures as described previously (Selvaraj et al., Life Sci Alliance 4(4):e202000886, 2021; Stauft et al., Virology 556:96-100, 2021). Tissues (lungs, trachea, and nasal turbinates) were fixed in 10% neutral buffered formalin overnight and then processed for paraffin embedding. The 5-μm sections were stained with hematoxylin and eosin for histopathological examinations. Images were scanned using an Aperio ImageScope. Blinded samples were graded by a licensed pathologist for the following twelve categories: consolidation, alveolar wall thickening, alveolar airway infiltrates, perivascular infiltrates, perivascular edema, type II pneumocyte hyperplasia, atypical pneumocyte hyperplasia, bronchiole mucosal hyperplasia, bronchiole airway infiltrates, proteinaceous fluid, hemorrhage, and vasculitis. Grading: 0=none, 1=mild, 2=moderate, 3=severe. A graph was prepared by summing up the score in each category.

### Virus Titration

Tissue culture infectious dose 50% (TCID₅₀) assays were done described previously (Selvaraj et al., Life Sci Alliance 4(4):e202000886, 2021; Stauft et al., Virology 556:96-100, 2021) for initial nasal wash titrations post-inoculation. In brief, Vero E6 cells were plated the day before infection into 96 well plates at 1.5×10⁴ cells/well. On the day of the experiment, serial dilutions of 20 μl nasal wash samples were made in media and a total of six to eight wells were infected with each serial dilution of the virus. After 48-hour incubation, cells were fixed in 4% PFA followed by staining with 0.1% crystal violet. The TCID₅₀ was then calculated using the formula: log (TCID₅₀)=log(do)+log (R) (f+1). Where do represents the dilution giving a positive well, f is a number derived from the number of positive wells calculated by a moving average, and R is the dilution factor.

For focus-forming assay (FFA), nasal wash, BALF, and lung homogenate samples were 10-fold serially diluted in 96-well plates and dilutions were added to 96-well black-well plates for fluorescent FFA in H1299-hACE2 cells (Stauft et al., J Infect Dis 227(2):202-205, 2023). After 1 hour, the Tragacanth gum overlay (final concentration 0.3%) was added. Cells were incubated at 37° C. and 5% CO₂ for 1 day, then fixed with 4% PFA, followed by staining of cells with primary rabbit anti-N Wuhan-1 antibody (Genscript) overnight followed by secondary anti-rabbit Alexa-488 conjugated antibody and DAPI staining. The infectious titers were then counted using Gen5 software on a Cytation7 machine and calculated and plotted as focus forming units per milliliter (FFU/ml). Limits of detection for the FFA were set based on the minimum detectable titer given 1 FFU at the lowest dilution (10⁻¹) and inoculation volume (50 μl). Any values below the lower limit (200 FFU/ml) were arbitrarily assessed as 100 FFU/ml for statistical analysis.

### Sars-CoV-2 Neutralization Assay

Samples were serially diluted 2-fold in 5% FBS DMEM and mixed with 100 PFU of SARS-CoV-2 in a 96-well plate at 37° C. for 1 hour. Sample:virus mixtures were then added to confluent H1299-hACE2 cells in 96-well plates. Cells were infected for 1 hour before the inoculum was removed and washed three times with DPBS. A second overlay containing 1.2% Tragacanth gum, 2×MEM, 5% FBS, and DMEM was added to the plate. Cells were incubated at 37° C. for 1 day, then fixed with 4% PFA, followed by staining of cells with primary rabbit anti-SARS-CoV-2 N antibody (Genscript U739BGB150-5) overnight followed by secondary anti-rabbit Alexa-488 conjugated antibody and 4′,6-diamidino-2-phenylindole (DAPI) staining. Plates were imaged on a Cytation7 (Agilent), and foci were counted using Gen5 software. For the neutralization assays, recombinant LY-CoV555 (Bamlanivimab) mixed with WA1/2020 (Wang et al., Proc Nal Acad Sci USA 118:(29):e2102775118, 2021) was included as a positive control. The 50% endpoint neutralization titers were determined as the reciprocal of the highest dilution providing≤half of the number of foci obtained from the negative control well (plain DMEM mixed with 100 PFU virus).

### Measurement of Antibody by ELISA

The preparation of SARS-CoV-2 RBD antigen in a baculovirus expression system and its use in ELISA were previously described (Meseda et al., NPJ Vaccines 6:145, 2021). ELISAs were performed with slight modifications. Briefly, Immulon 2^{HB} plates were coated with recombinant RBD protein at 1 μg/mL overnight at 4° C. Test serum samples were pre-diluted in assay diluent (PBS containing 0.05% Tween-20 [PBST] and 10% fetal bovine serum), followed by serial two-fold dilutions of each sample in duplicates across the plate. A starting dilution of 1:160, 1:80, and 1:20 was used for serum (IgA and IgG), BALF (IgG) and nasal wash and BALF (IgA) samples, respectively. Plates were incubated with the test serum samples for 2 hours at 37° C. After rigorous plate washes in a microplate washer, plates were incubated with anti-hamster antibodies. For IgG ELISA, a 1:4000 dilution of an HRP-conjugated goat anti-hamster IgG (6060-05, Southern Biotech, Birmingham, Alabama) was added to assay wells. For IgA ELISA, a rabbit anti-hamster IgA antibody [sandwich antibody; (cat. #sab 3001a) Brookwood Biomedical, Jemison, Alabama] was added to assay wells at 1:4000 dilution and plates incubated for 1 hour at 37° C. Unbound sandwich antibody was washed off and a 1:4000 dilution of an HRP-conjugated goat anti-rabbit IgG (4030-05, Southern Biotech, Birmingham, Alabama) was added to assay plates. In both IgG and IgA ELISAs, incubation with HRP-conjugated secondary antibodies lasted 1 hour after which plates were rigorously washed to remove unbound antibodies. The ABTS/H₂O₂ peroxidase substrate (SeraCare, Gaithersburg, Maryland) was added to assay wells and plates left at room temperature for 20 to 30 minutes. Color development was stopped by adding 1% SDS and OD₄₀₅ values were captured on the VersaMax microplate reader with Softmax Pro 7 software (Molecular Devices). In the IgG ELISA, the mean OD4 ms values of PBS treatment groups were subtracted from the mean OD₄₀₅ values from other treatment groups and the assay endpoint was a mean OD₄₀₅ value 0.05 (after background subtraction). In the IgA ELISA, the assay endpoint was a mean OD₄₀₅ value 0.02 of duplicate wells. Antibody titer was defined as the reciprocal of the highest dilution of a sample at which the mean OD₄₀₅ value for duplicate wells was 0.02 (IgA) or 0.05 after background subtraction (IgG).

### IFN-Gamma ELISpot

Hamster interferon gamma (IFN-γ) enzyme-linked immunosorbent spot (ELISpot) analysis was performed using the Hamster IFN-γ ELISpotBASIC (MABTECH Mabtech 3102-2H, Nacka Strand, Sweden) kit according to the manufacturer's instructions. MSIP Plates (Millipore) were washed 5 times with sterile water, coated with mAb (MTH21) and incubated overnight at 4° C. Coated plates were washed 5 times with 1×PBS, blocked for 30 minutes (room temperatures) with supplemented RPMI 1640 (GibcoBRL) containing 10% heat inactivated FBS, 1% 100× penicillin, streptomycin, and L-Glutamine solution (GibcoBRL). Freshly isolated splenocytes (2.5×10⁵) were seeded in each well and stimulated for 45-48 hours at 37° C. with SARS CoV-2 spike protein peptide pools (2 μg/ml each peptide) (BEI Catalog No. NR-52418) or nucleocapsid protein peptide pools (2 μg/ml each peptide) (BEI Catalog No. NR-52419) prepared in serum free RPMI 1640. Negative and positive plate controls were medium or 2 μg/ml concanavalin A (ConA, Sigma-Aldrich), respectively. Plates were incubated with 1 μg/ml mAb (MTH29-biotin) for 2 hours, and then 1 hour with streptavidin-HRP, and finally developed after adding TMB substrate (product No. 3651-10). Distinct spots typically emerge within 20 minutes. After drying, spots were counted using a BioTek Cytation 7 imaging reader (Agilent) and analysis software Gen5 Version No. 3.11. ELISpot data was analyzed in Microsoft Excel. The average number of spots from two negative wells (unstimulated cells) was subtracted from peptide pools stimulated wells for each plate. Results were expressed as difference in spot forming cells (SFC)/10⁶ PBMC between negative control and peptide pools stimulations conditions. Results were plotted using GraphPad Prism 9.

### Lung Immunofluorescence Analyses

FFPE lung sections 4 μm thick were dewaxed, rehydrated, and heat-treated in a microwave oven for 15 minutes in 10 mM Tris/1 mM EDTA buffer (pH 9.0). After cooling for 30 minutes at room temperature, heat-retrieved sections were blocked in PBST with 2.5% bovine serum albumin (BSA) for 30 minutes at RT followed by overnight incubation at 4° C. with primary antibodies in 1% BSA. Primary antibodies used included SARS nucleocapsid protein (NP) (1:800, Sino Biologicals, 40143-MM05), MX1 (Proteintech, 13750-1-AP), prosurfactant protein C (ProSPC) (1:200, EMD Millipore, AB3786), Iba1 (1:100, Abcam, ab5076), RAGE (1:400, Abcam, ab216329), and E-cadherin (ECAD) (Abcam, ab219332). Sections were rinsed and incubated with ALEXA FLUOR 488 (A-21206) and ALEXA FLUOR 647-conjugated secondary antibodies (A-31571, A-21447) for 1 hour at RT (ThermoFisher, Waltham, MA). Nuclei were counterstained with Hoechst 33342. For double labeling experiments, primary antibodies were mixed and incubated overnight at 4° C. For negative controls, sections were incubated without the primary antibody or mouse and rabbit isotype antibody controls. Sections stained with conjugated secondary antibodies alone showed no specific staining. Whole slide fluorescence imaging was performed using a Hamamatsu NanoZoomer 2.0-RS whole-slide digital scanner equipped with a 20× objective and a fluorescence module #L11600. Analysis software NDP.view2 was used for image processing (Hamamatsu Photonics, Japan). Immunofluorescence and differential interference images were also captured using an Axio Observer Z1 inverted microscope (Carl Zeiss. Thornwood, NY) equipped with an Axiocam 506 monochrome camera, an ApoTome.2 optical sectioning system, and a Plan-Apochromat 63×/1.4NA oil immersion with WD=0.19 and Plan-Apochromat 20×/0.8 objective lens. Digital image post-processing and analysis were performed using the ZEN 2 ver. 2.0 imaging software. Images were constructed from Z-stack slices collected at 0.48 μm intervals (4 pin thickness in total) and visualized as maximum intensity projections in orthogonal mode. For semiquantitative analysis of NP staining, high resolution whole-slide digital images of each lung section were acquired and the NDP.view2 software was used to measure the NP-stained area as a percentage of total area of the section. For TUNEL staining, sections were deparaffinized, hydrated, and pretreated with Proteinase K, followed by EDTA, distilled 1-O wash, and BSA blocking. Sections were then incubated in a reaction mixture (TdT, dUTP, and buffer), washed, and incubated with anti-digoxigenin antibody. Sections were then visualized with alkaline phosphatase-ImmPACT Vector Red and counterstained with hematoxylin.

### Sars-CoV-2 Pseudovirus Production and Infection Assay

Human codon-optimized cDNA encoding SARS-CoV-2 S glycoprotein of the WA1/2020 and the BA.5 variant (with C-terminal 19 amino acids deleted) were synthesized by GenScript and cloned into eukaryotic cell expression vector pcDNA 3.1 between the BamHI and XhoT sites. Pseudovirions were produced by co-transfection of Lenti-X 293T cells with psPAX2, pTRIP-GFP, and SARS-CoV-2 S expressing plasmid using Lipofectamine 3000. The supernatants were harvested at 48 and 72 hours post-transfection and filtered through 0.45-μm membranes. Infection was done as previously described (Liu et al., Cell Rep 40:111359, 2022). Forty-eight house post-infection, cells were fixed and imaged by Leica Stellaris 5 confocal microscope. Nuclei were stained with 4′,6-diamidino-2-phenylindole (DAPI).

### Statistical Analysis

One-way ANOVA or Student t-test was used to calculate statistical significance through GraphPad Prism (9.1.2) software for Windows, GraphPad Software, San Diego, California USA.

### Example 7: Mucosal and Systemic Immunogenicity of Nsp1-K164a/H165A

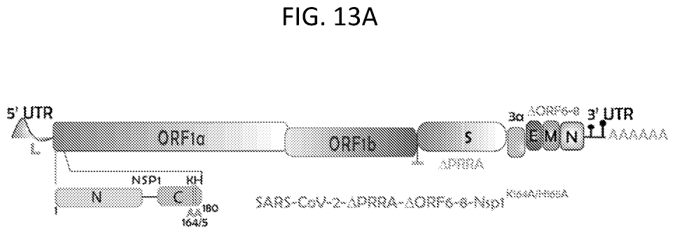

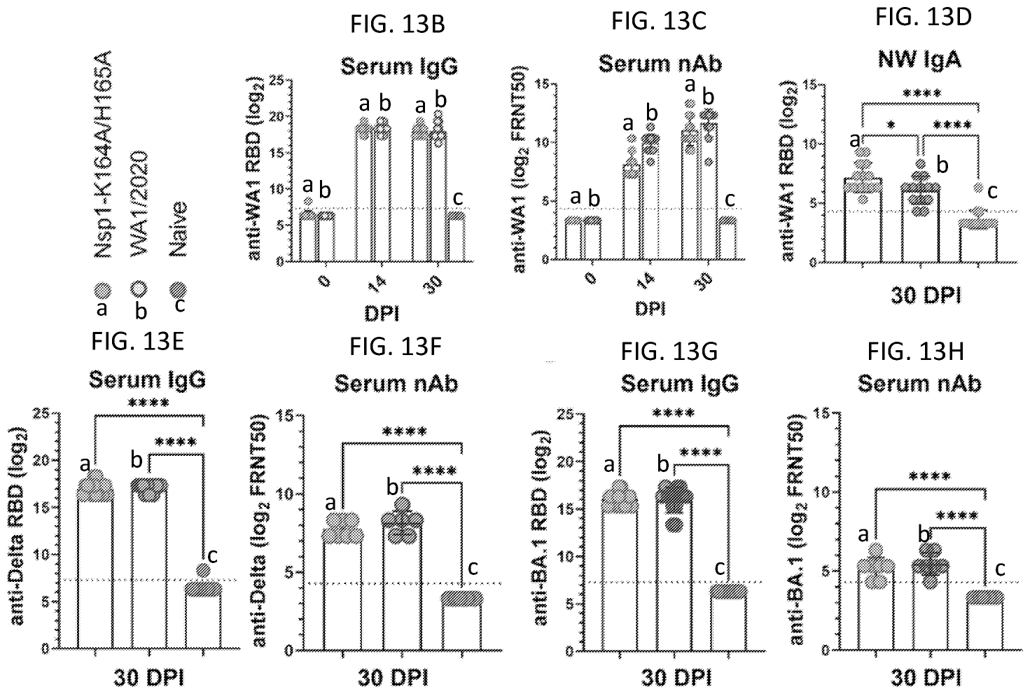

Anti-SARS-CoV-2 spike Immunoglobulin G (IgG) and IgA in serum samples and nasal washes from Syrian hamsters after intranasal inoculation of 100 PFU Nsp1-K164A/H165A or the wild-type WA1/2020 virus was assessed. Sera collected at 14- and 30-days post-infection (DPI) from both groups contained high titers of IgG antibodies specific for the WA1/2020 receptor binding domain (RBD) (FIG. 13B). Serum neutralizing antibody (nAb) titers increased from 0 to 14 DPI (p<0.0001, mixed-effects analysis) and then from 14 to 30 DPI (p<0.0001) for both groups of animals. Geometric mean titers (GMT) of serum nAbs against WA1/2020, indicated by 50% focus forming reduction neutralization titers (FRNT₅₀), were 2100 (interquartile range, IQR, 3840) and 3169 (IQR 2560) at 30 DPI for Nsp1-K164A/H165A and WA1/2020 groups, respectively (FIG. 13C). At 30 DPI, IgA titers detected in nasal washes were significantly higher (p<0.0001) in Nsp1-K164A/H165A (GMT 145, IQR 240) and WA1/2020 (GMT 68, IQR 80) groups compared to naive controls (FIG. 13D). In this experiment, there was 2-fold higher IgA titers (p=0.0336) in the Nsp1-K164A/H165A group than in WA1/2020 infected animals. When the same sera were measured for anti-Delta variant RBD IgG, a 2-fold reduction was observed in both the WA1/2020 (GMT 139621 versus 250997) and Nsp1-K164A/H165A (GMT 127911 versus 243465) groups in comparison to WA1/2020 RBD-specific IgG titers (FIG. 13E in comparison to FIG. 13B). Concomitantly, serum nAb titers against Delta variant dropped by nearly 10-fold compared to those against the ancestral WA1/2020 for both Nsp1-K164A/H165A (GMT 215, IQR 160) and WA1/2020 (GMT 285, IQR 240) groups (FIG. 13F in comparison to FIG. 13C). Similarly, serum Omicron BA.1 RBD-specific IgG titers, in comparison to WA1/2020 RBD-specific IgG titers, decreased by nearly 4-fold in Nsp1-K164A/H165A-vaccinated animals (GMT 243465 versus 70613) and in WA1/2020-infected (GMT 250997 versus 62749) hamsters at 30 DPI (FIG. 13G compared to FIG. 13B). NAb against the BA.1 variant reached titers just above the limit of detection from both the Nsp1-K164A/H165A-vaccinated group (GMT 36, IQR 20, a >50-fold reduction compared to nAb titers against the ancestral WA1/2020) and the WA1/2020-infected group (GMT 44.2, IQR 40, a >70-fold reduction compared to nAb titers against the ancestral WA1/2020) (FIG. 13H in comparison to FIG. 13C).

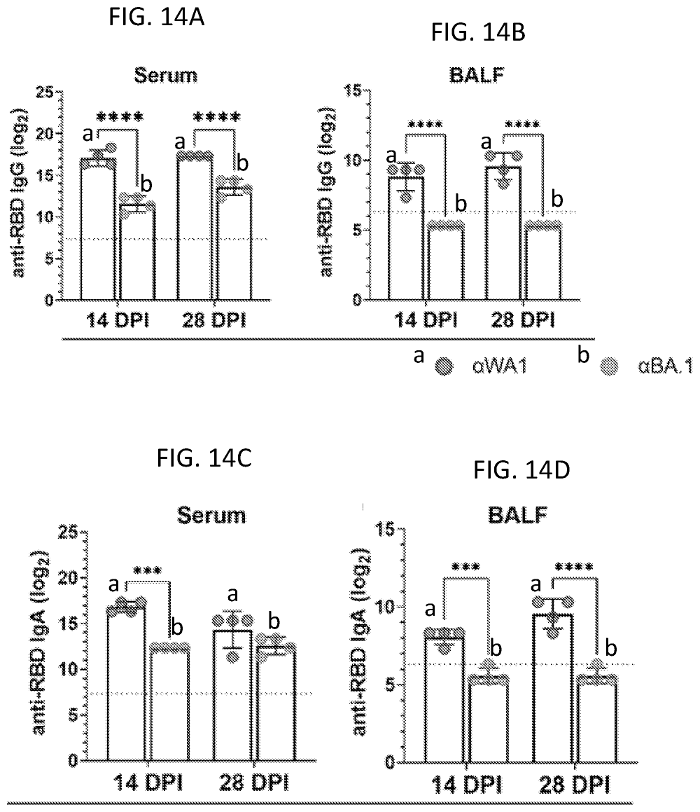

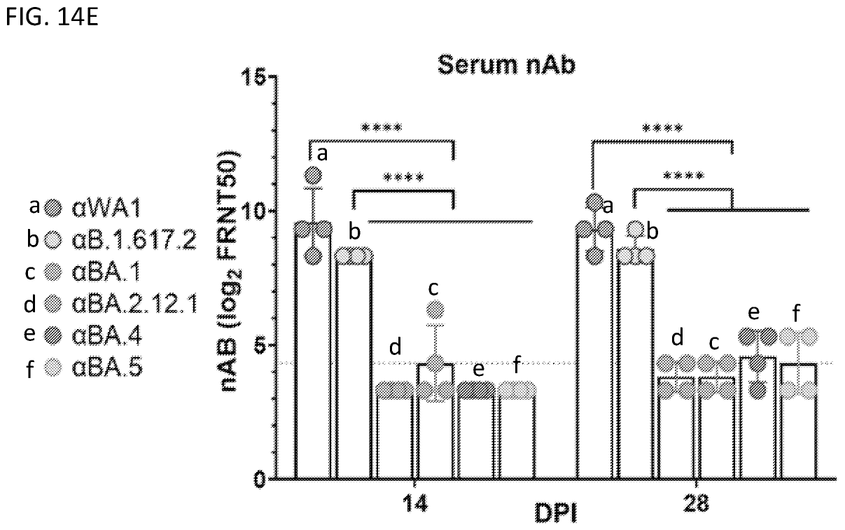

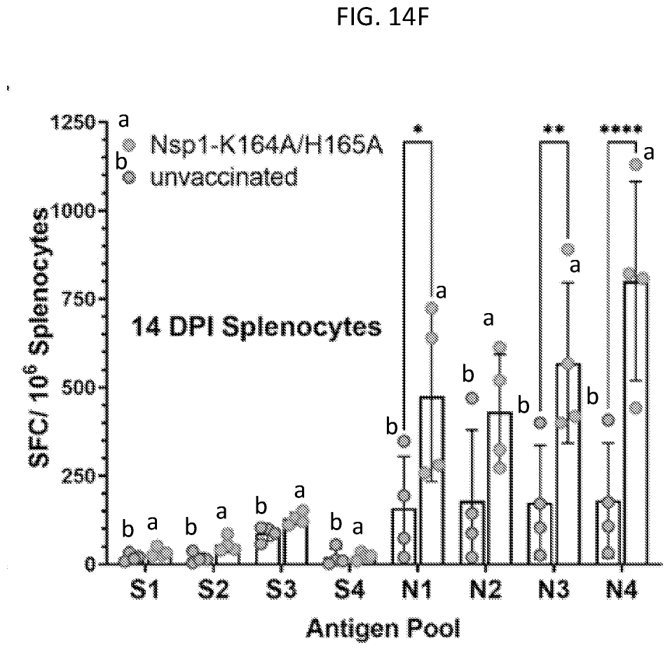

To ensure that the loss of serum neutralization antibody to Omicron BA.1 was not due to the low vaccine dose (100 PFUT), Syrian hamsters were inoculated with 10⁴ PFU Nsp1-K164A/H165A and characterized antibody responses to various Omicron subvariants. Because secretory IgA (SIgA) is important for antiviral immunity in the lung (Oh et al., Sci Immunol 6(66):eabj5129, 2021), anti-SARS-CoV-2 spike IgG and IgA in serum and then in bronchoalveolar lavage fluid (BALF) were assessed. At 14 and 28 DPI, serum anti-RBD IgG titers against WA1/2020 (GMT of 137772 and 163840, respectively) were 20-fold higher (p<0.0001) than those against BA.1 variant (GMT of 3044 and 12177, respectively) (FIG. 14A). BALF collected on 14 and 28 DPI contained detectable levels of IgG specific for ancestral RBD (GMT of 452 and 761, respectively), however, IgG titers specific for BA.1 RBD were below the limit of detection (FIG. 14B). Serum WA1/2020 RBD-specific IgA titers decreased by 5.65-fold from 14 (GMT 115852, IQR 81920) to 28 DPI (GMT 20480, IQR 28800) (p=0.0233, Sidak's multiple comparisons test), whereas serum anti-BA.1 RBD-specific IgA titers stayed at comparable levels at 14 and 28 DPI (FIG. 14C). In BALF, BA.1 RBD-specific IgA was undetectable at 14 and 28 DPI, but WA1/2020 RBD-specific IgA was detected at both 14 DPI (p=0.0003) and 28 DPI (p<0.0001) (FIG. 14D). GMT nAb titers against WA1/2020 were 761 (IQR 1680, 14 DPI) and 640 (IQR 720, 28 DPI) with greater than 32-fold reductions when measured against Omicron subvariants BA.1, BA.2.12.1, BA.4, and BA.5 (FIG. 14E). Lastly, the cellular immunity elicited by Nsp1-K164A/H165A vaccination was assessed. Significant induction of IFNγ-secreting cells was observed at 14 DPI in splenocytes harvested from vaccinated group pulsed with nucleocapsid antigen pools N1 (p=0.0096). N3 (p=0.0096), and N4 (p<0.0001) by ELISpot assays (FIG. 14F). Taken together, a single dose of Nsp1-K164A/H165A, when administered intranasally, induced IgA/IgG against SARS-CoV-2 spike protein in both respiratory tract and in circulation. However, these anti-spike antibodies are variant-specific and subject to evasion by Omicron variants.

### Example 8: Efficacy of Nsp1-K164a/H165A Against Challenge with Delta and Omicron Variants

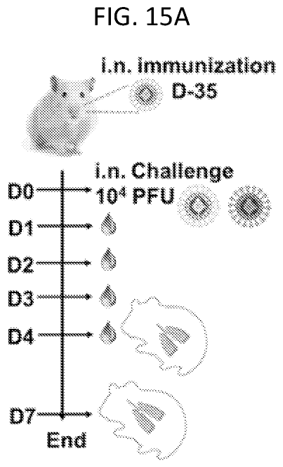

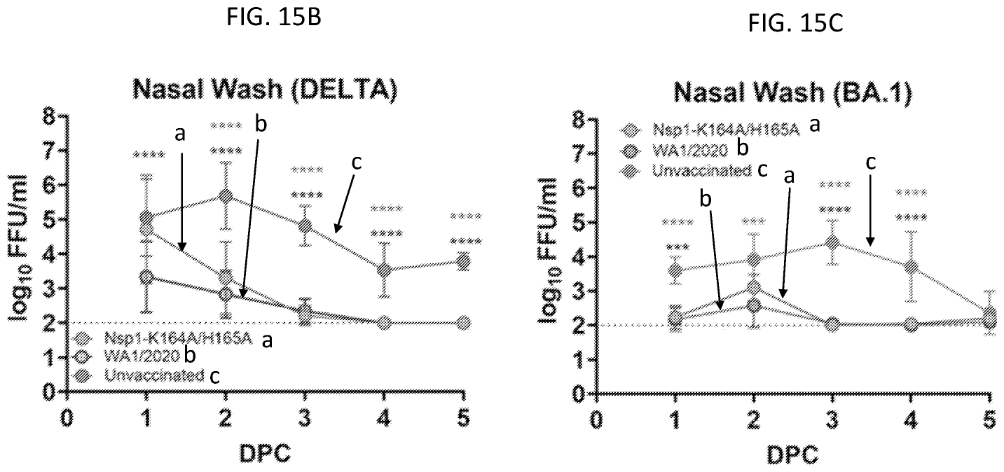

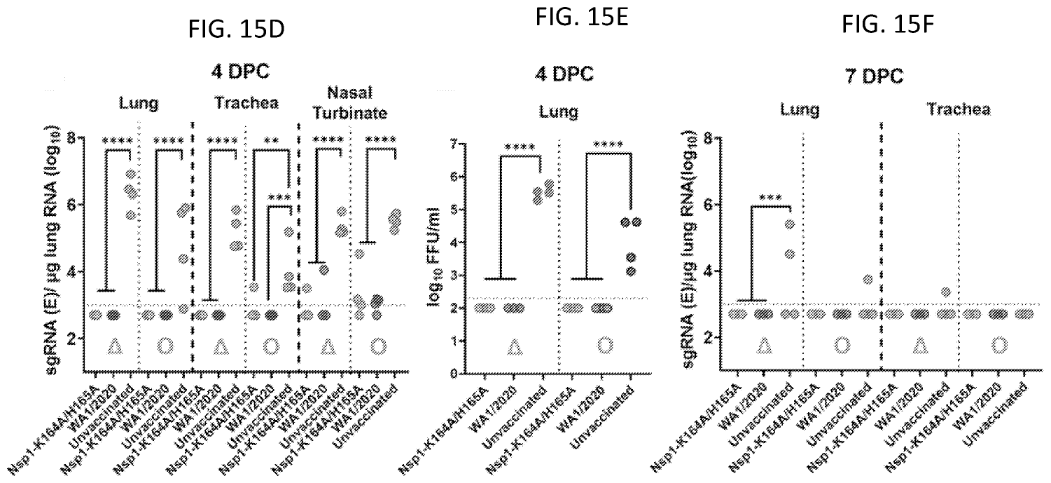

To assess whether intranasal administration of Nsp1-K164A/H165A offers protection against VOCs, male, 5-month-old Syrian hamsters were inoculated with 100 PFU of Nsp1-K164A/H165A (n=14) or WA1/2020 (n=14) by the intranasal route (derived from the study depicted in FIG. 13). At 35 days-post-infection (DPI), the vaccinated and convalescent hamsters (n=6-7 per group) along with unvaccinated naïve hamsters (n=8) were challenged with 10⁴ PFU of a Delta isolate (hCoV-19/USA/MD-HP05647/2021) or an BA.1 Omicron isolate (hCoV-19/USA/HI-CDC-4359259-001/2021). On 4 and 7 days-post-challenge (DPC), animals were euthanized to collect tissues for analyses of viral replication and pathology (FIG. 15A). Nasal wash samples were also collected from each hamster following challenge. We noted a significant reduction of infectious virus (p<0.0001) in nasal wash samples at 2, 3, 4, and 5 DPC by Delta in animals inoculated with Nsp1-K164A/H165A or WA1/2020 compared to unvaccinated controls (FIG. 15B). Similarly, Nsp1-K164A/H165A vaccinated and WA1/2020 convalescent hamsters also exhibited reduced nasal viral load at 1 (WA1/2020, p=<0.0001; Nsp1-K164A/H165A, p=0.0001), 2 (WA1/2020 only, p=0.0002), 3 (p<0.0001), and 4 (p<0.0001) DPC by BA.1. By 5 DPC, infectious virus titers declined to baseline in all BA1 challenged animals (FIG. 15C). At 4 DPC, subgenomic viral RNA (sgRNA) of the envelope (E) protein, a hallmark of viral replication, was readily detectable in the lungs, trachea, and nasal turbinates of Delta- and BA.1-challenged unvaccinated animals (FIG. 15D). By contrast, sgRNA levels were reduced by more than 4400-fold (p=<0.0001) in the lungs, around 300-fold in trachea (p<0.0001), and 300-fold in nasal turbinates (p<0.0001) of Nsp1-K164A/H165A vaccinated and WA1/2020 convalescent animals after challenge with the Delta variant. As to BA.1 challenged animals, the reduction of sgRNA was greater than 100-fold in lungs (p<0.0001), trachea (p=0.0041), and nasal turbinates (p<0.0001) at 4 DPC in both Nsp1-K164A/H165A vaccinated and WA1/2020 convalescent animals compared to the unvaccinated group. Infectious viral titers in lung homogenates also diminished below the limit of detection in Nsp1-K164A/H165A vaccinated and WA1/2020 convalescent groups following either Delta or Omicron BA.1 challenge at 4 DPC (FIG. 15E). At 7 DPC, sgRNA levels in the lungs were below the limit of detection except for the unvaccinated/challenged hamsters (FIG. 15F). Taken together, these results indicate that intranasal vaccination with Nsp1-K164A/H165A effectively reduces viral loads in both upper and lower respiratory tract of Syrian hamsters upon heterologous virus challenge.

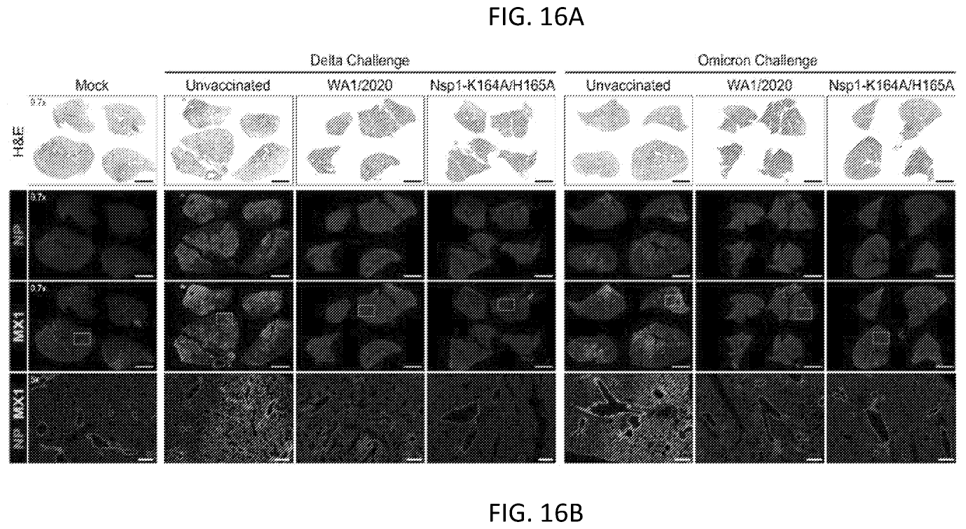

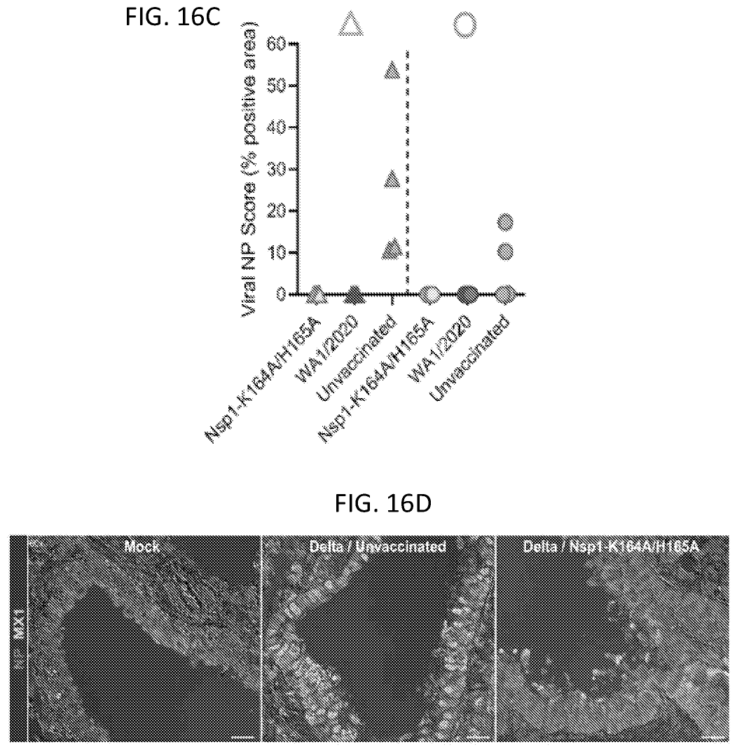

To further examine the presence of viral antigens and host innate immune activation, lung sections from uninfected (mock) or challenged hamsters were stained with hematoxylin and eosin (H&E) or immunostained for viral nucleocapsid protein (NP) and myxovirus resistance 1 (MX1), an interferon-induced antiviral host response marker (Halfmann et al., J Infect Dis 225:282-286, 2022; Frere et al., Sci Transl Med 14:eabq3059, 2022) (FIG. 16). Lungs from Delta-challenged unvaccinated hamsters at 4 DPC showed widespread immune infiltrates and regions of viral NP deposition characterized by prominent staining of the epithelial lining of infected bronchioles accompanied by intense staining of surrounding alveolar epithelium (FIGS. 16A-16B). Two of the four BA.1-challenged unvaccinated animals at 4 DPC showed NP deposition with a similar staining pattern (FIGS. 16B-16C). Delta- and BA.1-challenged unvaccinated groups also showed increased MX1 immunoreactivity in these NP-positive lung regions, which is particularly evident in the bronchiolar epithelium. Vaccination with Nsp1-K164A/H165A or infection with WA1/2020 blocked NP deposition and MX1 upregulation in the Delta- and BA.1-challenged groups (FIGS. 16B-16C). High resolution imaging of representative lung sections from Delta-challenged unvaccinated hamsters highlighted the pronounced upregulation of MX1 in nuclear and cytoplasmic compartments of infected bronchiolar epithelial cells and the attenuation in Nsp1-K164A/H165A-vaccinated hamsters (FIG. 16D). Altogether, the absence of NP staining and MX1 upregulation implies that the challenge virus, whether it is the Delta or Omicron BA.1 variant, failed to establish infection in the lungs of Nsp1-K164A/H165A-vaccinated and WA1/2020-convalescent hamsters.

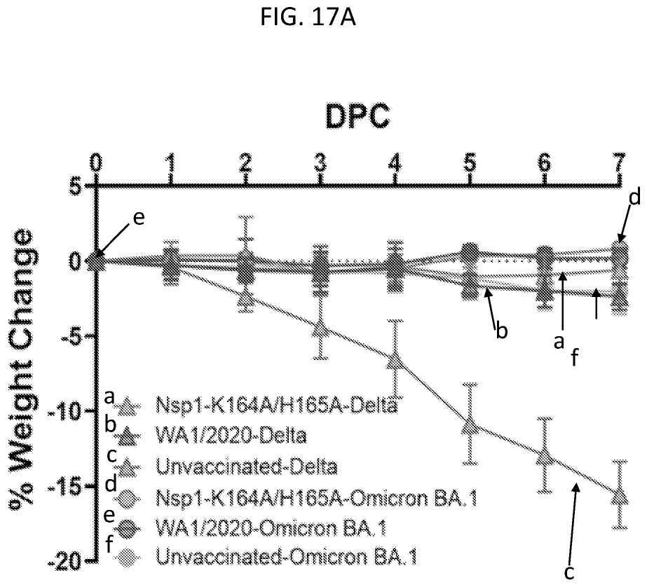

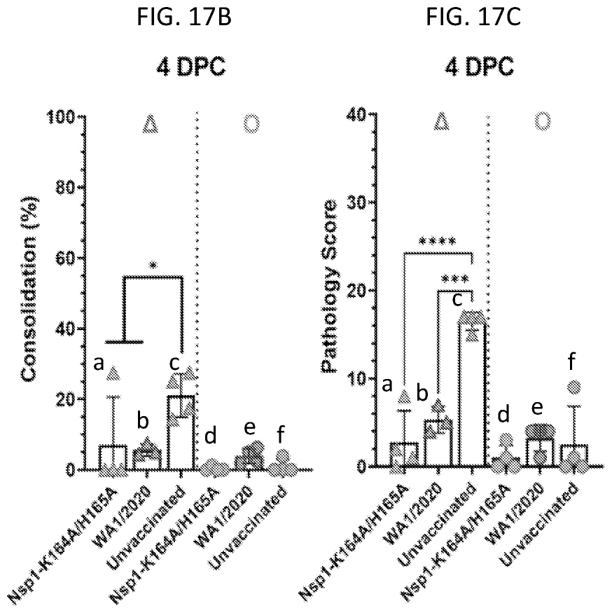

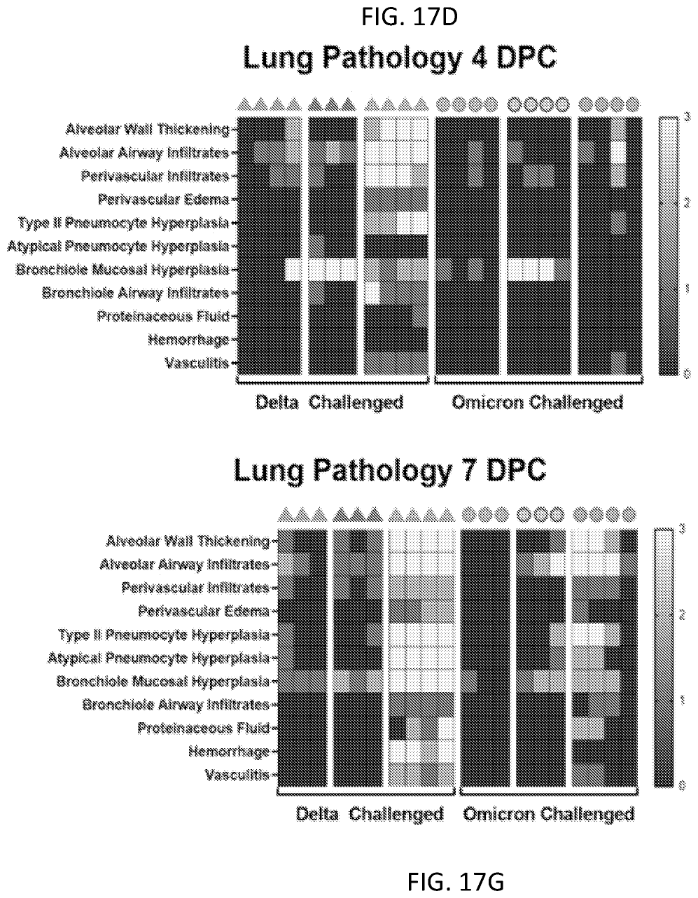

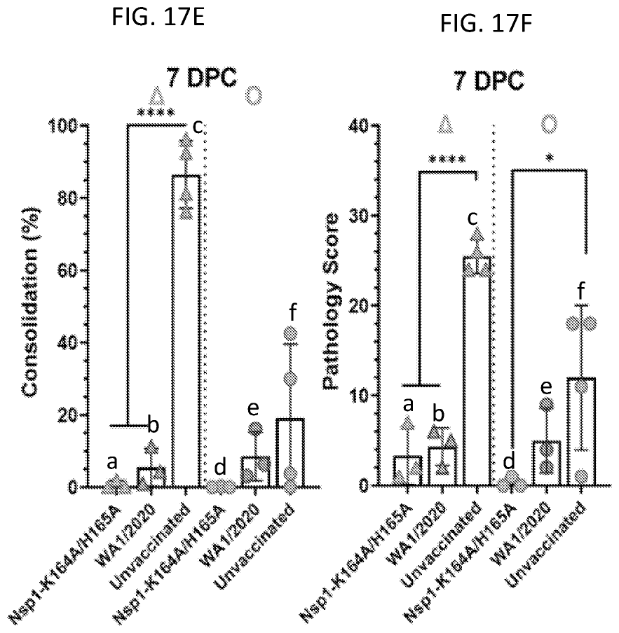

Among the Delta-challenged hamsters, only the unvaccinated group (n=8) significantly lost body weight over the course of 7 days (FIG. 17A). By contrast, none of the three groups lost weight after BA.1 challenge (FIG. 17A). At 4 DPC, the percent of lung consolidation in Delta variant-challenged hamsters was significantly reduced in WA1/2020-(p=0.0352, n=3) and Nsp1-K164A/H165A-inoculated groups (p=0.0386, n=4) compared to unvaccinated controls (FIG. 17B). Accumulated pathology scores were also significantly lower at 4 DPC in Delta-challenged WA1/2020 (p=0.0002) and Nsp1-K164A/H165A groups (p<0.0001) as opposed to the unvaccinated- and Delta-challenged animals (FIGS. 17B-17C). Differing from the Delta variant, Omicron BA.1 challenge resulted in low pathology overall, with minimal consolidation (FIG. 17B) and low pathology scores at 4 DPC (FIGS. 17C-17D). At 7 DPC, among Delta-challenged hamsters, both Nsp1-K164A/H165A and WA1/2020 inoculated groups (n=3) had significantly (p<0.0001) reduced consolidation compared to unvaccinated controls (FIG. 17E). Following BA.1 challenge, only two animals in the unvaccinated group had >20% lung consolidation at 7 DPC (FIG. 17E), however, lung pathology was evident in 3 out of 4 animals within this group (FIGS. 17F-17G). Again, nearly no lung pathology was detected from Nsp1-K164A/H165A-vaccinated animals (n=3, p=0.0195).

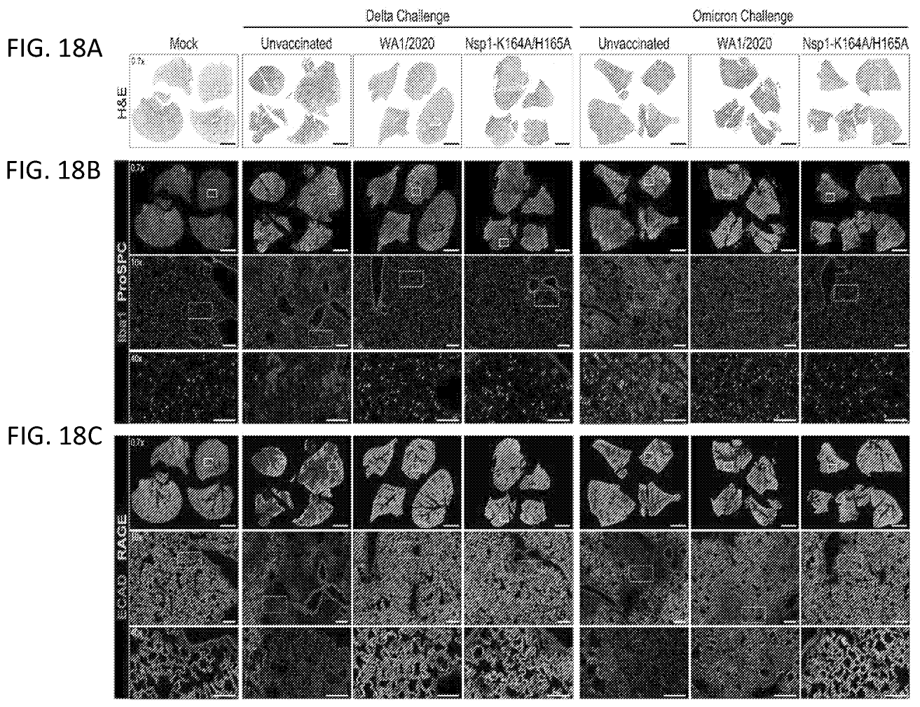

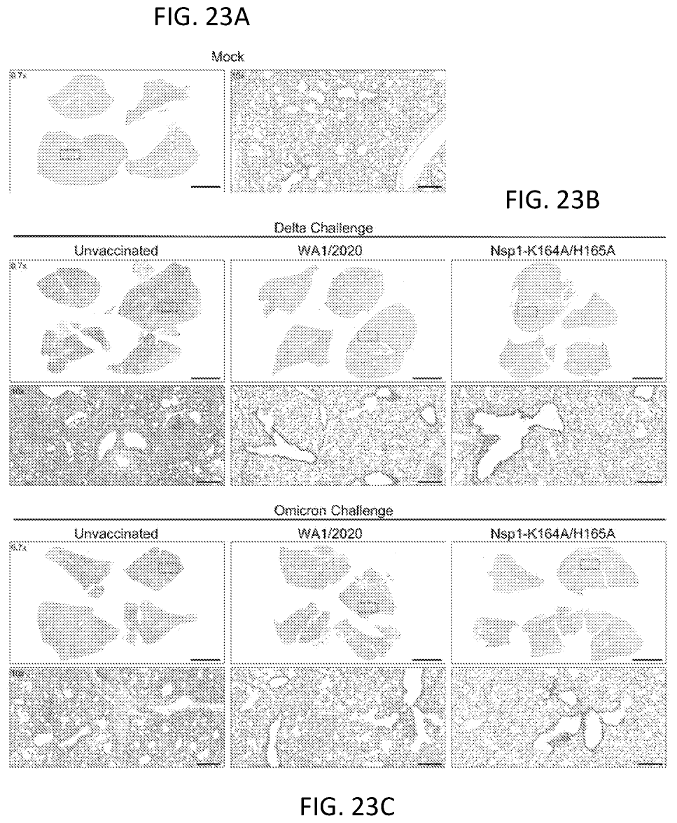

To further characterize these histopathological changes using specific markers of inflammation and epithelial damage, serial lung sections from uninfected hamsters (mock), unvaccinated hamsters, and WA1/2020-convalescent and Nsp1-K164A/H165A-vaccinated hamsters were immunostained for Iba1 (a marker of macrophages), prosurfactant protein C (ProSPC, a marker of AT2 cells), RAGE (a marker of AT1 cells), and E-cadherin (a marker of intercellular epithelial junctions). At 7 DPC, lungs from all four Delta-challenged unvaccinated hamsters and two of the four BA.1-challenged unvaccinated hamsters showed regions of consolidation by routine H&E that corresponded with areas containing extensive accumulation of Iba1-expressing macrophages (FIGS. 18A-18B) and increased terminal deoxynucleotidyl transferase dUTP nick end labeling (TUNEL) (FIG. 23). These consolidated regions also showed marked reduction of ProSPC-expressing AT2 cells and pronounced loss of alveolar RAGE expression along the borders of AT1 cells. Distinct areas of epithelial cell loss and Iba1-positive macrophage consolidation formed around E-cadherin-labeled bronchioles that were presumably subject to virus attack (FIG. 18C). Increased E-cadherin staining also identified hyperplastic epithelium in consolidated spaces that was particularly prominent in Delta-challenged unvaccinated lungs. Vaccination with Nsp1-K164A/H165A attenuated Iba1-positive macrophage consolidation and protected against the loss of ProSPC and RAGE expression in Delta- and BA.1-challenge groups (FIGS. 18A-18C). Prior infection with WA1/2020 also protected against epithelial damage in the Delta-challenge group.

### Example 9: Transmission of Nsp1-K164A/H165A in Syrian Hamsters

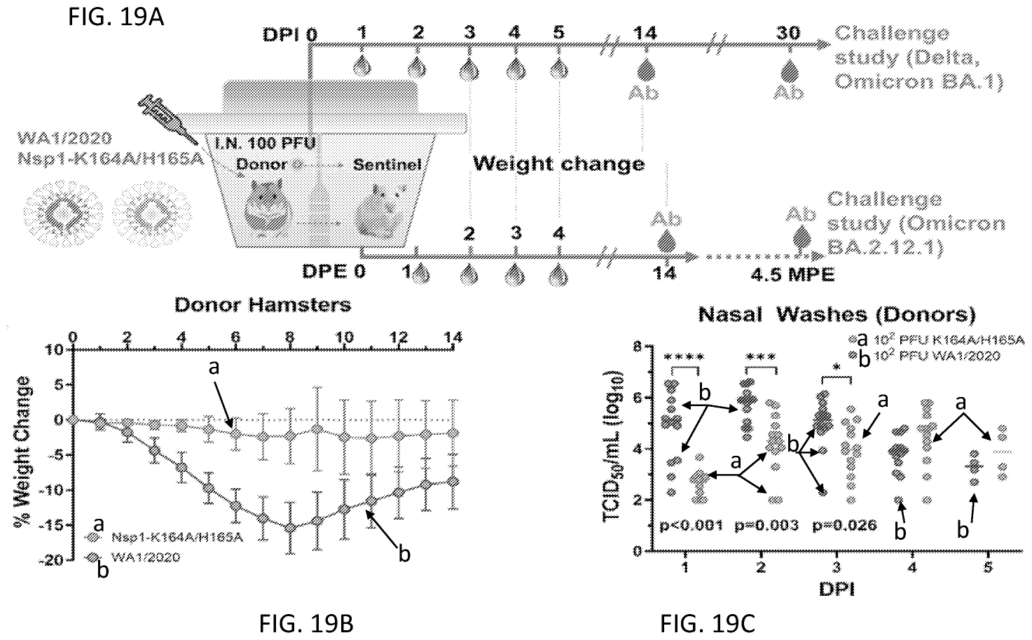

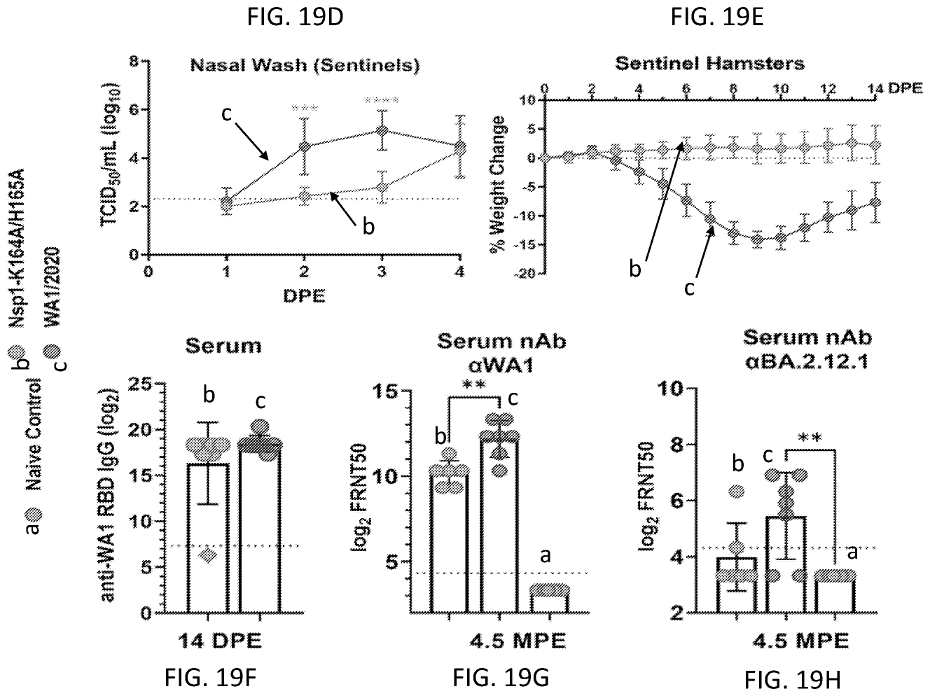

To characterize the transmissibility of Nsp1-K164A/H165A, an additional study was performed in an airborne transmission model (FIG. 19A) in which two Syrian hamsters in the same cage are separated from each other by a customized, perforated metal divider that prevents physical contact while permitting air exchange.

First, donor hamsters (male, 4-month-old, n=14) were inoculated with 100 PFU of WA1/2020 or Nsp1-K164A/H165A, which was previously shown to be immunogenic and protective against WA1/2020 challenge. Significant body weight loss was observed in WA1/2020 infected hamsters at days 3-9 (p<0.0001), 10-11 (p<0.001), and 12-14 (p<0.01) DPI and one had to be euthanized due to presentation of severe clinical signs (hypothermia, hunched posture, lethargy) at 7 DPI (FIG. 19B). By contrast, no significant weight loss was observed in Nsp1-K164A/H165A vaccinated animals (n=14). WA1/2020 inoculated hamsters also exhibited significantly higher infectious viral loads in nasal wash samples at 1, 2, 3 DPI than Nsp1-K164A/H165A inoculated group (1 DPI, 158-fold, p<0.0001; 2 DPI, −33-fold, p=0.0003; and 3 DPI, 10-fold, p=0.0105) (FIG. 19C).

At 1 DPI, 7 sentinel hamsters were paired individually with either a WA1/2020 or a Nsp1-K164A/H165A inoculated hamster in cages with dividers. Nasal washes were collected from the sentinel hamsters from 1-4 days post-exposure (DPE) and seroconversion was determined after two weeks to confirm infection. Nasal wash infectious virus titers from Nsp1-K164A/H165A-exposed sentinels were undetectable at 1 DPE and then significantly lower at 2 DPE (>100-fold, p=0.0006) and 3 DPE (>290-fold, p<0.0001) compared to those from WA1/2020-exposed sentinels (FIG. 19D). At 4 DPE, there was no statistically significant difference between sentinel groups although 2 of the Nsp1-K164A/H165A sentinels had nasal wash titers below the detection limit (200 TCID₅₀/mL). Overall, the transmission of Nsp1-K164A/H165A to sentinel hamsters exhibited a delayed kinetics. No weight loss was evident in the sentinel hamsters exposed to Nsp1-K164A/H165A, however, WA1/2020-exposed sentinels experienced weight loss at days 4-5 (p<0.05), 6 (p=0.0005), 7-12 (p<0.0001), 13 (p=0.0003), and 14 (p=0.0016) DPE with a maximum mean weight loss of 15.6% on 9 DPE (FIG. 19E). By 14 DPE all exposed sentinel animals had high levels of anti-RBD IgG in serum (FIG. 19F) except one animal from the Nsp1-K164A/H165A-exposed sentinel group (animal ID WH363), which did not seroconvert (there was also no detectable infectious virus in nasal washes collected from this animal).

At 4.5 months after exposure (MPE), all seroconverted sentinel animals were tested for nAb titers against WA1/2020 and BA.2.12.1. Serum nAb titers against WA1/2020 from WA1/2020 exposed sentinel animals (GMT 4637, IQR 7680) were 4-fold higher (p=0.0026, unpaired t-test) than Nsp1-K164A/H165A-exposed sentinel hamsters (GMT 1140, IQR 960) (FIG. 19G). Neutralization of BA.2.12.1, however, was observed in only 2 animals from the Nsp1-K164A/H165A sentinel group and 5 animals from the WA1/2020 sentinel group prior to challenge (FIG. 19H).

### Example 10: Passively Vaccinated Sentinel Hamsters are Protected from BA.2.12.1 Challenge

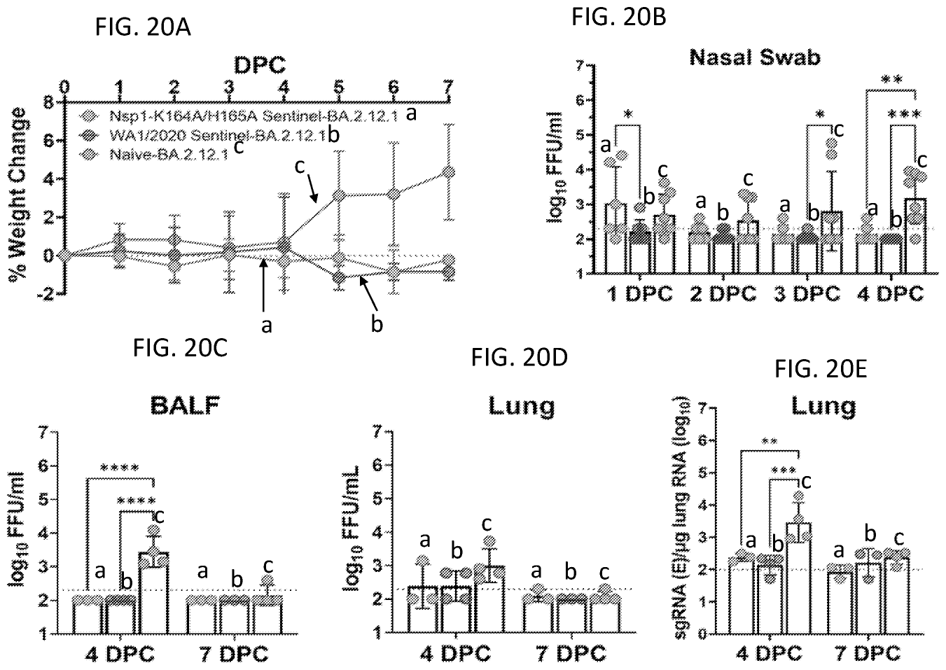

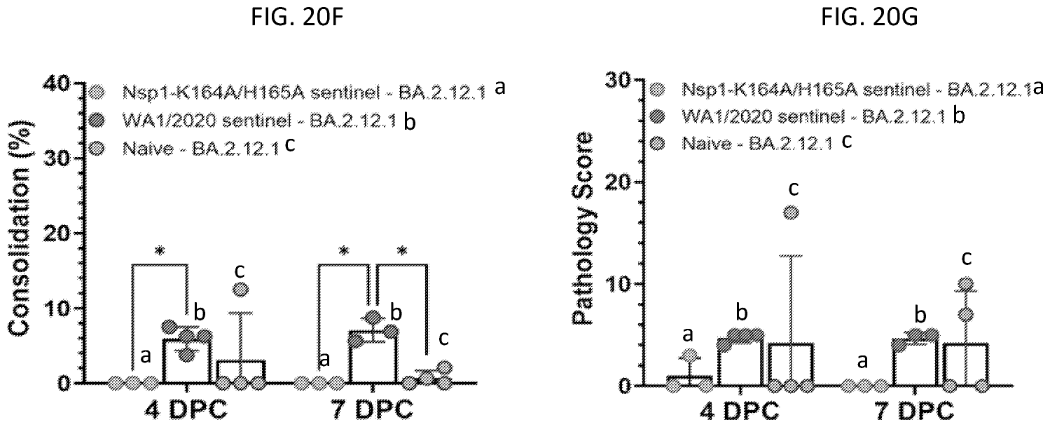

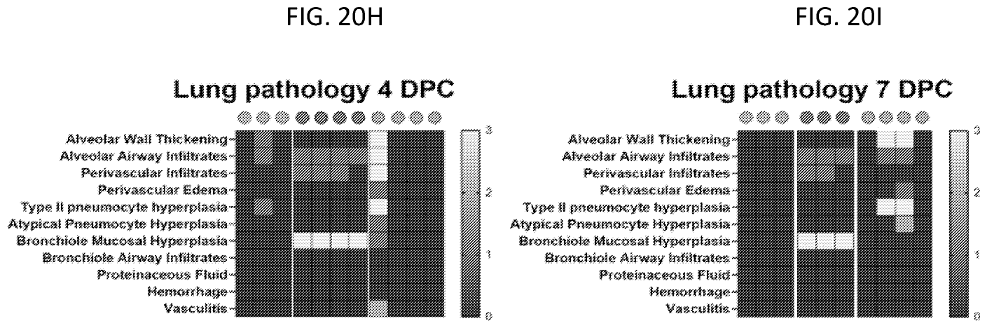

Four and half months after the initial exposure, seroconverted sentinel hamsters from FIG. 19 were challenged with 10⁴ FFU Omicron BA.2.12.1 by the intranasal route. Weight loss did not occur in any BA.2.12.1 challenged hamsters (FIG. 20A). Reduced nasal viral loads in WA1/2020-exposed sentinel hamsters (n=7) were noticed on 3 (p=0.0492) and 4 (p=0.0.0011) DPC compared to naïve hamsters that were challenged with BA.2.12.1 (FIG. 20B). Despite low or absent BA.2.12.1-specific nAb titers in Nsp1-K164A/H165A sentinel animals (FIG. 19H), nasal viral load of these animals was significantly lower compared to controls at 4 DPC (p=0.0094) (FIG. 20B). Infectious virus titers in BALF were at least one-log lower in WA1/2020 and Nsp1-K164A/H165A sentinel hamsters than in naïve controls at 4 DPC (p<0.0001) (FIG. 20C). Infectious virus titers in lung homogenates of the BA.2.121 infected control animals (n=4) were around 1000 FFU/mL at 4 DPC, whereas 2 out of 4 sentinel hamsters had no detectable infectious virus in the lungs (FIG. 20D). Consequently, log₁₀-transformed sgRNA copies detected in both Nsp1-K164A/H165A (p=0.0043) and WA1/2020 sentinel hamster lungs (p=0.0004) were at least one order of magnitude lower compared to controls at 4 DPC (FIG. 20E).

Histopathology analyses of fixed lungs revealed minimal consolidation at 4 DPC (n=4) and 7 DPC (n=3) in all groups. A slightly higher percentage of lung consolidation (FIG. 20F) was observed in WA1/2020-exposed sentinels compared to Nsp1-K164A/H165A (4 DPC, p=0.0490) or Nsp1-K164A/H165A sentinel hamsters (p=0.0269) and control animals (p=0.0326) at 7 DPC. The consolidation is most likely remnant pathology from the initial WA1/2020 infection, but not due to BA.2.12.1 challenge. There was no significant difference in pathology scores at 4 or 7 DPC between groups (FIG. 20G), although WA1/2020-exposed sentinel hamsters did show bronchiole mucosal hyperplasia at 4 DPC (FIG. 20H) and 7 DPC (FIG. 20I). Noticeable lung pathologies in naive animals after BA.2.12.1 challenge included alveolar wall thickening, airway infiltrates, and type II pneumocyte hyperplasia. By contrast, such pathologies were absent in the lungs of Nsp1-K164A/H165A exposed sentinel hamsters after challenge (FIG. 20H-20I). Altogether, Nsp1-K164A/H165A exposed sentinel hamsters were protected from BA.2.12.1 challenge in the lungs.

### Example 11: Nsp1-K164a/H165A Vaccination Protects Against BA.5 Challenge

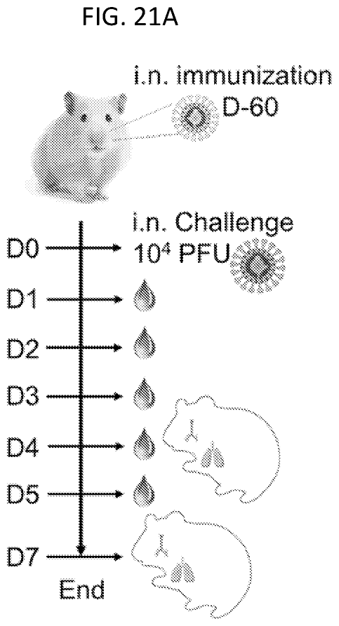

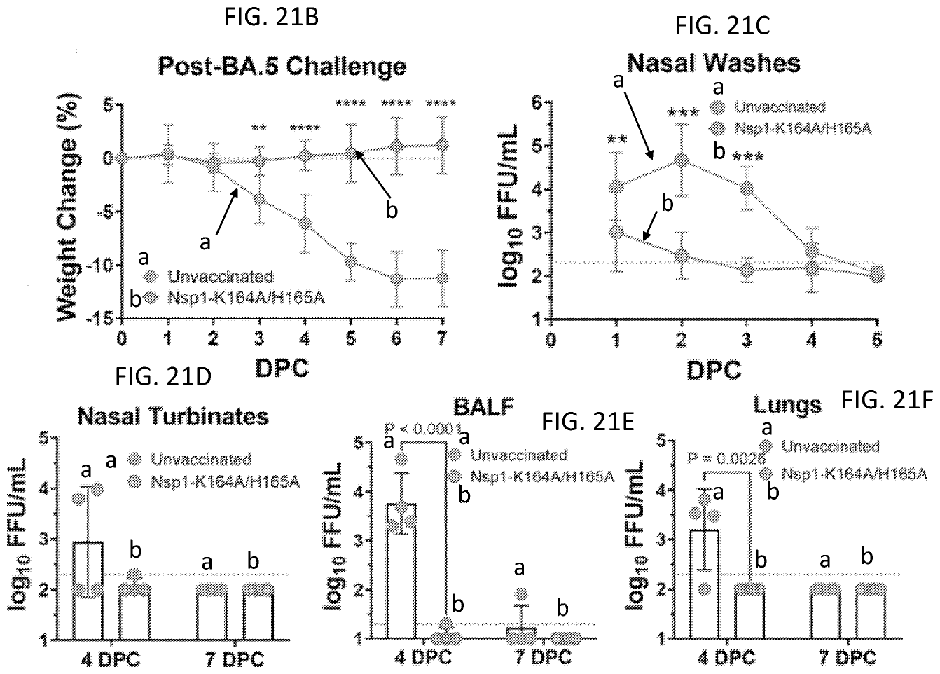

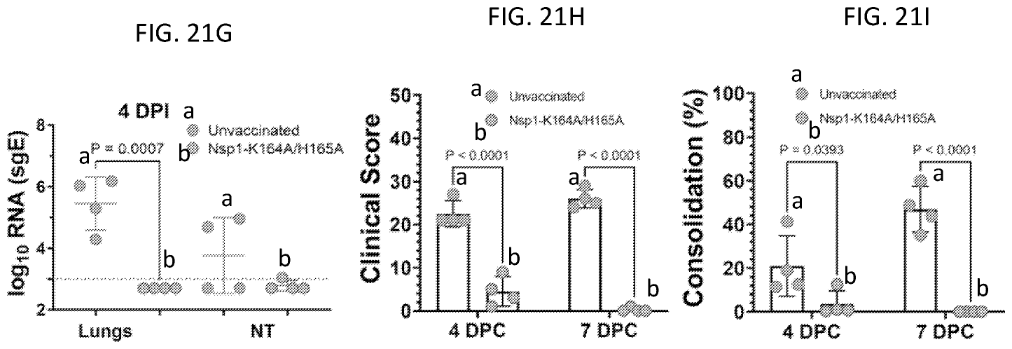

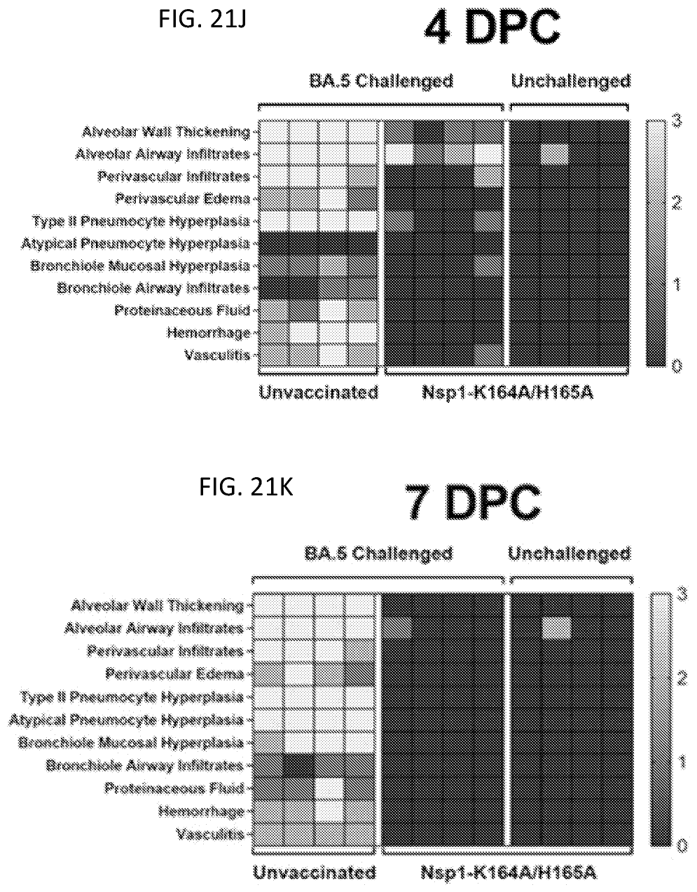

Because Omicron BA.1 and BA.2.12.1 only caused mild diseases in Syrian hamsters, another vaccination-challenge study was performed using a BA.5 isolate that induces more severe disease. Shown in FIG. 21A, sixty days after vaccination with 100 PFU Nsp1-K164A/H165A, Syrian hamsters were challenged with 10⁴ PFU BA.5 isolate. Unvaccinated animals rapidly lost weight over the next 7 days, whereas the body weight of vaccinated animals remained steady throughout the course of the study (FIG. 21B). Vaccinated animals had detectable virus from nasal wash samples collected at 1 DPC with minimal (at or below) infectious virus at subsequent timepoints. By contrast, unvaccinated animals shed at least 2 logs higher infectious virus in nasal wash samples from 1 to 3 DPC (FIG. 21C). Viral loads in nasal turbinates, BALF, and lungs, were nearly undetectable in vaccinated animals at 4 and 7 DPI (FIGS. 21D-21G). Lastly, Nsp1-K164A/H165A vaccinated hamsters also showed very little lung pathology at 4 and 7 DPC in comparison to unvaccinated animals (FIGS. 21H-21K). BA.4/BA.5 and some later Omicron subvariants are even more immune evasive than the initial BA.1 and BA.2 variants, but Nsp1-K164A/H165A LAV conferred protection against BA.5 both in the upper and lower respiratory tract in Syrian hamsters.

### Example 12: Variant-Specific Nsp1-K164a/H165A as a Booster

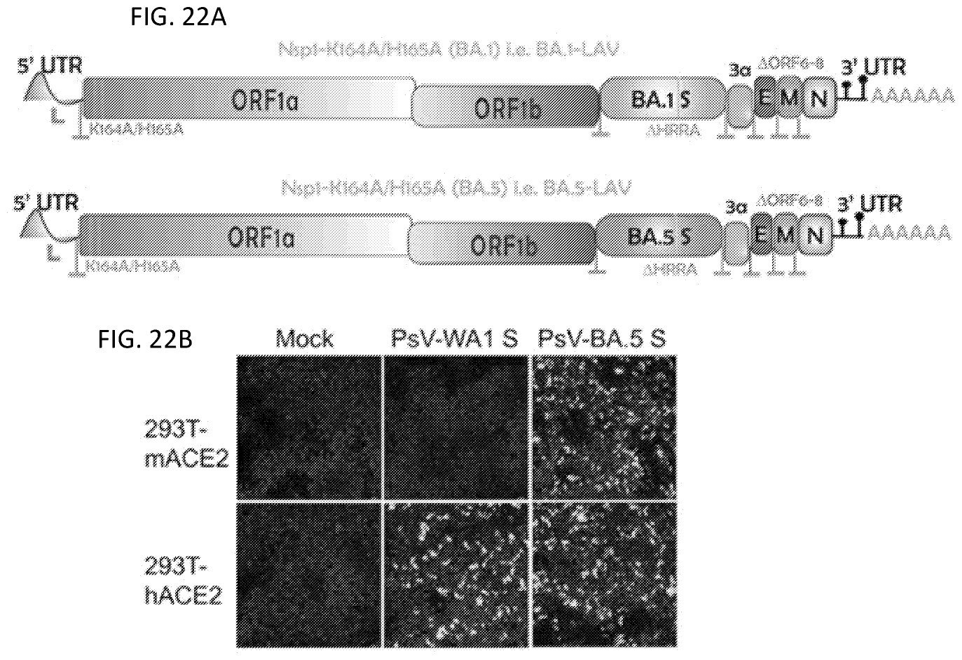

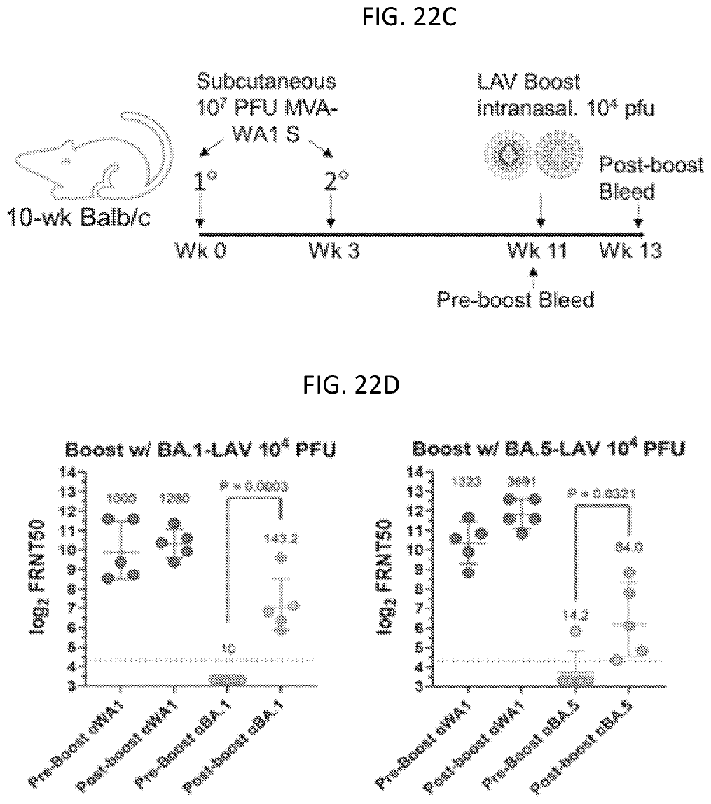

To test if Nsp1-K164A/H165A may be used as a booster vaccine candidate, two additional attenuated viruses were generated with the WA1 spike being replaced with either BA.1 or BA.5 spike protein, namely BA.1-LAV and BA.5-LAV, respectively (FIG. 22A). BA.1 enters cells expressing mouse ACE2 (mACE2) and infects laboratory mouse strains such as Balb/c (Liu et al., Cell Rep 40:111 359, 2022). In this study, it was also confirmed that BA.5 infects 293T cells expressing mACE2 (FIG. 22B). Hence, it was believed that BA.1-LAV and BA.5-LAV would infect Balb/c and induce a variant-specific antibody response. To test this possibility, Balb/c mice first received two doses of vaccinia virus Ankara vectors expressing full-length WA1 spike (MVA-S) (Meseda et al., NPJ Vaccines 6:145, 2021). After 8 weeks, 10⁴ PFU BA.1-LAV or BA.5-LAV were intranasally administered to these mice (FIG. 22C) (Liu et al., Cell Rep 40:111359, 2022). A dose of 10⁴ PFU was chosen to ensure that the candidate vaccine virus infects the mice with pre-existing immunity. The pre-boost sera contained high levels of nAbs against the original WA1 isolate, but nondetectable anti-BA.1 or anti-BA.5 nAb. Two weeks after one dose of BA.1-LAV and BA.5-LAV, the GMT values of corresponding nAb titers increased to 143 (IQR 387.6) and 84 (IQR 315.9), respectively (FIG. 22D).

It will be apparent that the precise details of the methods or compositions described may be varied or modified without departing from the spirit of the described aspects of the disclosure. We claim all such modifications and variations that fall within the scope and spirit of the claims below.

1. A live-attenuated severe acute respiratory syndrome coronavirus 2 (SARS-CoV-2), comprising a recombinant genome:
encoding a modified spike (S) protein with a deletion of the polybasic site (ΔPRRA) corresponding to residues 681-684 of the reference sequence set forth as SEQ ID NO: 2, and a modified non-structural protein 1 (Nsp1) with K164A and H165A substitutions corresponding to the reference sequence set forth as SEQ ID NO: 4; and
comprising a mutation that prevents expression of open reading frames (ORFs) 6, 7a, 7b and 8, and
wherein the live-attenuated SARS-CoV-2 is capable of infecting and replicating in mammalian cells.

2. The live-attenuated SARS-CoV-2 of claim 1, which is a Wuhan strain SARS-CoV-2, or a variant thereof from the Alpha, Beta, Delta, Gamma, Epsilon, Eta, Iota, Kappa, Mu, Zeta or Omicron lineage, comprising the recombinant genome.

3. The live-attenuated SARS-CoV-2 of claim 1, which is a SARS-CoV-2 variant of concern (VOC) comprising the recombinant genome.

4. The live-attenuated SARS-CoV-2 of claim 3, wherein the VOC is from the Delta lineage or the Omicron lineage.

5. The live-attenuated SARS-CoV-2 of claim 1, wherein the modified S protein is at least 90% identical to SEQ ID NO: 2 and has the deletion of the polybasic insert.

6. The live-attenuated SARS-CoV-2 of claim 5, wherein the amino acid sequence of the modified S protein comprises or consists of SEQ ID NO: 3.

7. The live-attenuated SARS-CoV-2 of claim 1, wherein the amino acid sequence of the modified Nsp1 is at least 90% identical to SEQ ID NO: 4 and includes the K164A and H165A substitutions.

8. The live-attenuated SARS-CoV-2 of claim 7, wherein the amino acid sequence of the modified Nsp1 comprises or consists of SEQ ID NO: 5.

9. The live-attenuated SARS-CoV-2 of claim 1, wherein the mutation that prevents expression of ORFs 6, 7a, 7b and 8 is a deletion of ORFs 6, 7a, 7b and 8.

10. An immunogenic composition comprising the live-attenuated SARS-CoV-2 of claim 1 and a pharmaceutically acceptable carrier.

11. The immunogenic composition of claim 10, further comprising an adjuvant.

12. The immunogenic composition of claim 10, formulated for intranasal administration.

13. A nucleic acid molecule or molecules comprising the complement of the recombinant genome of the live-attenuated SARS-CoV-2 of claim 1.

14. A collection of reverse genetics plasmids comprising the complement of the recombinant genome of the live-attenuated SARS-CoV-2 of claim 1.

15. A method of producing a live-attenuated SARS-CoV-2, comprising:
transfecting permissive cells with the reverse genetics plasmids of claim 14;
culturing the transfected cells under conditions sufficient to allow for replication of the attenuated SARS-CoV-2; and
isolating the attenuated SARS-CoV-2 from the cell culture.

16. An attenuated SARS-CoV-2 produced by the method of claim 15.

17. A kit, comprising:
the collection of reverse genetics plasmids of claim 14; and
transfection reagent(s), cultured cells, cell culture media and/or cell culture flasks.

18. A method of eliciting an immune response against SARS-CoV-2 in a subject, comprising administering to the subject an effective amount of the live-attenuated SARS-CoV-2 of claim 1, thereby eliciting an immune response against SARS-CoV-2 in the subject.

19. The method of claim 18, wherein the live-attenuated SARS-CoV-2 or the immunogenic composition is administered intranasally.

20. The method of claim 18, wherein the effective amount of the live-attenuated SARS-CoV-2 or the immunogenic composition is administered in a single dose.

21. The method of claim 18, wherein the live-attenuated SARS-CoV-2 or the immunogenic composition is administered as part of a prime-boost immunization protocol.

22. The method of claim 21, wherein the live-attenuated SARS-CoV-2 or the immunogenic composition is administered as both the prime dose and the boost dose.

23. The method of claim 21, wherein the live-attenuated SARS-CoV-2 or the immunogenic composition is administered as the prime dose and a second SARS-CoV-2 vaccine is administered as the boost dose.

24. The method of claim 21, wherein the live-attenuated SARS-CoV-2 or the immunogenic composition is administered as the boost dose and a second SARS-CoV-2 vaccine is administered as the prime dose.

25. The method of claim 23, wherein the second SARS-CoV-2 vaccine is administered intramuscularly.
